(19) United States
(12) Patent Application Publication
(10) Pub. No.: US 2022/0214362 A1
(43) Pub. Date: Jul. 7, 2022

(54) EVALUATING BIOMARKERS ALONG WITH ADVANCED MAGNETIC RESONANCE IMAGING PROCEDURES IN A HUMAN SUBJECT THAT HAS SUSTAINED OR MAY HAVE SUSTAINED A HEAD INJURY

(71) Applicants: ABBOTT LABORATORIES, Abbott Park, IL (US); GENERAL ELECTRIC COMPANY, Schenectady, NY (US)

(72) Inventors: Beth McQuiston, Abbott Park, IL (US); Luca Marinelli, Schenectady, NY (US); Saul Datwyler, Abbott Park, IL (US)

(21) Appl. No.: 17/609,995

(22) PCT Filed: May 8, 2020
(86) PCT No.: PCT/US2020/032194

§ 371 Date: Nov. 9, 2021

Related U.S. Application Data
(60) Provisional application No. 62/846,243, filed on May 10, 2019.

(51) Int. Cl. G01N 33/68

(52) U.S. Cl. CPC G01N 33/6896; A61B 5/0042; G01N 2800/52

(57) ABSTRACT

Disclosed herein are methods that aid in the determination of whether to perform one or more advanced magnetic resonance imaging (MRI) procedures for a head injury by determining the presence or amount of one or more biomarkers in a sample obtained from the human subject. Also disclosed are methods of aiding in the diagnosis and evaluation of a human subject that has sustained or may have sustained an injury to the head, e.g., by assessing biomarker levels in combination with advanced MRI procedures. Further, also disclosed are methods of predicting or aiding in the prediction of the outcome of human subjects that have suffered a traumatic brain injury (TBI) as well as determining the course of treatment or efficacy of a course of treatment for a human subject who has suffered a TBI, e.g., by assessing biomarker levels in combination with advanced MRI procedures.

### RELATED APPLICATION INFORMATION

This application claims priority to U.S. Application No. 62/846,243, filed on May 10, 2019, the contents of which are herein incorporated by reference.

### TECHNICAL FIELD

The present disclosure relates to methods of evaluating a human subject to determine whether to perform one or more advanced magnetic resonance imaging (MRI) procedures for an actual or suspected head injury by determining the presence or amount of one or more biomarkers in a sample obtained from the human subject. Additionally, the present disclosure relates to methods of aiding in the diagnosis and evaluation of a human subject that has sustained or may have sustained an injury to the head by determining the presence or amount of one or more biomarkers in a sample obtained from the subject, obtaining data from the subject using at least one advanced magnetic resonance imaging procedure, and processing all or part of the information or data obtained from the biomarkers and the advanced magnetic resonance imaging procedure(s) using a computer-implemented method to diagnose and evaluate whether the subject has or has not suffered a head injury, and if the subject has suffered a head injury, determining the (i) pathophysiology of the head injury; (ii) the endotype of the head injury; (iii) whether the subject has, or more likely than not has, sustained a mild, moderate, severe, or moderate to severe TBI; or (iv) any combinations of (i)-(iii). Additionally, these methods can also be used to predict or as an aid in predicting the outcome of human subjects that have suffered a traumatic brain injury (TBI) as well as determining the course of treatment or efficacy of a course of treatment for a human subject who has suffered a TBI.

### BACKGROUND

More than 5 million mild traumatic brain injuries (TBIs) occur each year in the United States alone. Currently, there is no simple, objective, accurate measurement available to help in patient assessment. In fact, much of TBI evaluation and diagnosis is based on subjective data. Unfortunately, objective measurements such as head computerized tomography (CT) scan and or clinical measures such as Glasgow Coma Score (GCS) are not very comprehensive or sensitive in evaluating mild TBI. Moreover, head CT is unrevealing for the vast majority of the time for mild TBI, is expensive, and exposes the patient to unnecessary radiation. Additionally, a negative head CT does not mean the patient has been cleared from having a concussion; rather it just means certain interventions, such as surgery, are not warranted. Clinicians and patients need objective, reliable information to accurately evaluate this condition to promote appropriate triage and recovery. To date, limited data have been available for the use of biomarkers (e.g., such as S100β, UCH-L1, GFAP, etc.) in the acute care setting to aid in patient evaluation and management.

Mild TBI or concussion is much harder to objectively detect and presents an everyday challenge in emergency care units globally. Concussion usually causes no gross pathology, such as hemorrhage, and no abnormalities on conventional computed tomography scans of the brain, but rather rapid-onset neuronal dysfunction that resolves in a spontaneous manner over a few days to a few weeks. Approximately 15% of mild TBI patients suffer persisting cognitive dysfunction. There is an unmet need for mild TBI victims on scene, in emergency rooms and clinics, in the sports area and in military activity (e.g., combat).

Current algorithms for assessment of the severity of brain injury include Glasgow Coma Scale score and other measures. These measures may at times be adequate for relating acute severity but are insufficiently sensitive for subtle pathology which can result in persistent deficit. GCS and other measures also do not enable differentiation among types of injury and may not be adequate. Thus, patients grouped into a single GCS level may have vastly heterogeneous severity and type of injury. Because outcomes also vary accordingly, inappropriate classification undermines the integrity of a clinical trial. Improved classification of injury will enable more precise delineation of disease severity and type for TBI patients in clinical trials.

Additionally, current brain injury trials rely on outcome measures such as Glasgow Outcome Scale Extended, which capture global phenomena but fail to assess for subtle differences in outcome. Thus 30 consecutive trials for brain injury therapeutics have failed. Sensitive outcome measures are needed to determine how well patients have recovered from brain injury in order to test therapeutics and prophylactics.

### DESCRIPTION OF THE FIGURES

FIG. 1 shows a comparison of biomarker profiles of two mild TBI cases that demonstrate that levels of GFAP and UCH-L1 are higher at about 12 hours compared to about 21 hours post-injury, as further described in Example 1.

### SUMMARY

In some embodiments, the present disclosure relates to methods of evaluating a human subject to determine whether to perform an advanced magnetic resonance imaging (MRI) procedure for a head injury. The methods comprise the steps of:
a) performing an assay on one or more samples obtained from the subject after an actual or suspected injury to the head to measure or detect a level of one or more biomarkers of traumatic brain injury (TBI); and
b) determining whether to perform an advanced MRI procedure on the subject when the level of the one or more biomarkers in the one or more samples are unfavorable when compared to one or more reference levels of the one or more biomarkers and not performing an advanced MRI procedure on the subject when the level of the one or more biomarkers in the one or more samples are favorable when compared to one or more reference levels of the one or more biomarkers.

In the above methods, the subject has received a conventional MRI procedure or an advanced MRI procedure before or after the assay is performed. In one aspect, the subject has received a conventional MRI before the assay is performed. In another aspect, the subject has received an advanced MRI procedure before the assay is performed. In another aspect, the subject has received a conventional MRI after the assay is performed. In yet another aspect, the subject has received an advanced MRI procedure after the assay is performed.

In the above methods, the subject is suspected of having or is confirmed as having a head injury based on the conventional MRI procedure or advanced MRI procedure. In one aspect, the subject is suspected of having a head injury based on a conventional MRI procedure. In another aspect, the subject is confirmed as having a head injury based on a conventional MRI procedure. In yet another aspect, the subject is suspected of having a head injury based on an advanced MRI procedure. In still yet another aspect, the subject is confirmed as having a head injury based on an advanced MRI procedure.

In other aspects, the one or more biomarkers are selected from the group consisting of: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In some aspects, the one or more biomarkers comprises two or more biomarkers. In other aspects, the one or more biomarkers comprises three or more biomarkers. In yet other aspects, the one or more biomarkers comprises four or more biomarkers. In still yet other aspects, the one or more biomarkers comprises five or more biomarkers. In still yet other aspects, the one or more biomarkers comprises six or more biomarkers. In still yet other aspects, the one or more biomarkers comprises seven or more biomarkers. In still yet other aspects, the one or more biomarkers comprises eight or more biomarkers. In still yet other aspects, the one or more biomarkers comprises nine or more biomarkers. In still yet other aspects, the one or more biomarkers comprises ten or more biomarkers.

In yet other aspects, the one or more biomarkers are: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

In other aspects, the advanced MRI procedure is gradient echo magnetic resonance imaging, echo planar magnetic resonance imaging, spin echo magnetization resonance imaging, diffusion MRI, resting-state functional MRI, magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

In other aspects, the one or more samples is taken from the subject within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, with about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of the actual or suspected injury to the head.

In other aspects, the sample is selected from the group consisting of a whole blood sample, a serum sample, a cerebrospinal fluid sample, and a plasma sample. In some aspects, the sample is a whole blood sample. In other aspects, the sample is a serum sample. In yet other aspects, the sample is a cerebrospinal fluid sample. In still yet other aspects, the sample is a plasma sample. In still yet other aspects, the sample is a serum or plasma sample. In still yet other aspects, the sample is a whole blood, serum or plasma sample. In still other aspects, the sample is a whole blood or serum sample. In still yet other aspects, the sample is a whole blood or plasma sample.

In still yet other aspects, the sample is obtained after the subject sustained an injury to the head caused by physical shaking, blunt impact by an external mechanical or other force that results in a closed or open head trauma, one or more falls, explosions or blasts or other types of blunt force trauma. In yet other aspects, the sample is obtained after the subject has ingested or been exposed to a chemical, toxin or combination of a chemical and toxin. In some aspects, the chemical or toxin is fire, mold, asbestos, a pesticide, an insecticide, an organic solvent, a paint, a glue, a gas, an organic metal, a drug of abuse or one or more combinations thereof. In still yet other aspects, the sample is obtained from a subject that suffers from an autoimmune disease, a metabolic disorder, a brain tumor, hypoxia, a virus, meningitis, hydrocephalus or combinations thereof.

The above methods can be carried out on any subject without regard to factors selected from the group consisting of the subject's clinical condition, the subject's laboratory values, the subject's classification as suffering from mild, moderate, severe, or moderate to severe traumatic brain injury, the subject's exhibition of low or high levels of one or more biomarkers, and the timing of any event wherein said subject may have sustained an injury to the head.

In some aspects, the assay is an immunoassay. In some aspects, the assay is an immunoassay and the sample is a whole blood, serum or plasma sample. In some aspects, the assay is an immunoassay and the sample is a whole blood sample. In yet other aspects, the assay is an immunoassay and the sample is a plasma sample. In still yet other aspects, the assay is an immunoassay and the sample is a serum sample.

In some aspects, the assay is a clinical chemistry assay. In some aspects, the assay is a clinical chemistry assay and the sample is a whole blood, serum or plasma sample. In some aspects, the assay is a clinical chemistry assay and the sample is a whole blood sample. In yet other aspects, the assay is clinical chemistry assay and the sample is a plasma sample. In still yet other aspects, the assay is a clinical chemistry assay and the sample is a serum sample.

In some aspects, the assay is a single molecule detection assay. In some aspects, the assay is a single molecule detection assay and the sample is a whole blood, serum or plasma sample. In some aspects, the assay is a single molecule detection assay and the sample is a whole blood sample. In yet other aspects, the assay is single molecule detection assay and the sample is a plasma sample. In still yet other aspects, the assay is a single molecule detection assay and the sample is a serum sample.

In yet other embodiments, the present disclosure relates to methods of aiding in the diagnosis and evaluation of a human subject that has sustained or may have sustained an injury to the head. The methods comprise the steps of:
a) performing at least one assay on one or more samples taken from the subject to measure or detect a level of one or more biomarkers of TBI;
b) determining the amount of said one or more biomarkers in the one or more samples;
c) performing at least one advanced MRI procedure on the subject;
d) obtaining data from the procedure using at least one computer-implemented method;
e) generating a TBI diagnosis for the subject using the amount of the one or more biomarkers determined in step b) and the data obtained in step d); and
f) determining whether the subject has sustained or not sustained a head injury based on the TBI diagnosis.

In the above methods, if the subject is determined to have sustained a head injury, the method further comprises assessing: (a) the pathophysiology of the head injury; (b) the endotype of the head injury; (c) whether the subject has, or more likely than not, sustained a mild, moderate, severe, or moderate to severe TBI; or (d) any combinations of (a)-(c). In some aspects, if the subject is determined to have sustained a head injury, the methods further comprise assessing the pathophysiology of the head injury. In other aspects, if the subject is determined to have sustained a head injury, the methods further comprise assessing the endotype of the head injury. In yet other aspects, if the subject is determined to have sustained a head injury, the methods further comprise assessing whether the subject has, or more likely has not, sustained a mild, moderate, severe or moderate to severe TBI. In still yet other aspects, if the subject is determined to have sustained a head injury, the methods further comprise assessing the pathophysiology of the head injury, the endotype of the head injury and whether the subject has, or more likely has not, sustained a mild, moderate, severe or moderate to severe TBI.

In the above methods, in some aspects, a first TBI diagnosis is generated for the subject based on the amount of the one or more biomarkers determined in step b). In some aspects, additionally, a second TBI diagnosis is generated based on the data obtained in step d). In some aspects, it is determined whether the subject has not sustained a head injury based on a combination of the first TBI diagnosis and the second TBI diagnosis.

In some aspects of the above methods, the advanced MRI procedure identifies the location of the TBI in the brain of the subject. In yet other aspects, the subject is predicted of having an unfavorable outcome based on the location of the TBI.

In some aspects, the pathophysiology of the head injury is determined by assessing whether the subject has or is suffering from at least one of cerebral hypofusion or hyperfusion, cerebral ischaemia, hyperaemia, impaired cerebrovascular CO₂-reactivity, a cerebral vasospasm, brain tissue hypoxia, oedema, oxidative stress, cell death or combinations thereof.

In some aspects, the endotype of the head injury is determined by determining (i) whether the injury is axonal, neuronal or astroglial; (ii) whether there is microvascular injury; (iii) whether there is proteinopathy; (iv) whether is there inflammation; or (v) any combinations thereof.

In some aspects, the above methods further comprise monitoring the subject. Alternatively, or in addition to, in some aspects, the above methods involve treating the subject.

In other aspects, the one or more biomarkers are selected from the group consisting of: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In some aspects, the one or more biomarkers comprises two or more biomarkers. In other aspects, the one or more biomarkers comprises three or more biomarkers. In yet other aspects, the one or more biomarkers comprises four or more biomarkers. In still yet other aspects, the one or more biomarkers comprises five or more biomarkers. In still yet other aspects, the one or more biomarkers comprises six or more biomarkers. In still yet other aspects, the one or more biomarkers comprises seven or more biomarkers. In still yet other aspects, the one or more biomarkers comprises eight or more biomarkers. In still yet other aspects, the one or more biomarkers comprises nine or more biomarkers. In still yet other aspects, the one or more biomarkers comprises ten or more biomarkers.

In yet other aspects, the one or more biomarkers are: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

In other aspects, the advanced MRI procedure is gradient echo magnetic resonance imaging, echo planar magnetic resonance imaging, spin echo magnetization resonance imaging, diffusion MRI, resting-state functional MRI, magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

In other aspects, the one or more samples is taken from the subject within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, with about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of the actual or suspected injury to the head.

In other aspects, the sample is selected from the group consisting of a whole blood sample, a serum sample, a cerebrospinal fluid sample, and a plasma sample. In some aspects, the sample is a whole blood sample. In other aspects, the sample is a serum sample. In yet other aspects, the sample is a cerebrospinal fluid sample. In still yet other aspects, the sample is a plasma sample. In still yet other aspects, the sample is a serum or plasma sample. In still yet other aspects, the sample is a whole blood, serum or plasma sample. In still other aspects, the sample is a whole blood or serum sample. In still yet other aspects, the sample is a whole blood or plasma sample.

In still yet other aspects, the sample is obtained after the subject sustained an injury to the head caused by physical shaking, blunt impact by an external mechanical or other force that results in a closed or open head trauma, one or more falls, explosions or blasts or other types of blunt force trauma. In yet other aspects, the sample is obtained after the subject has ingested or been exposed to a chemical, toxin or combination of a chemical and toxin. In some aspects, the chemical or toxin is fire, mold, asbestos, a pesticide, an insecticide, an organic solvent, a paint, a glue, a gas, an organic metal, a drug of abuse or one or more combinations thereof. In still yet other aspects, the sample is obtained from a subject that suffers from an autoimmune disease, a metabolic disorder, a brain tumor, hypoxia, a virus, meningitis, hydrocephalus or combinations thereof.

The above methods can be carried out on any subject without regard to factors selected from the group consisting of the subject's clinical condition, the subject's laboratory values, the subject's classification as suffering from mild, moderate, severe, or moderate to severe traumatic brain injury, the subject's exhibition of low or high levels of one or more biomarkers, and the timing of any event wherein said subject may have sustained an injury to the head.

In some aspects, the assay is an immunoassay. In some aspects, the assay is an immunoassay and the sample is a whole blood, serum or plasma sample. In some aspects, the assay is an immunoassay and the sample is a whole blood sample. In yet other aspects, the assay is an immunoassay and the sample is a plasma sample. In still yet other aspects, the assay is an immunoassay and the sample is a serum sample.

In some aspects, the assay is a clinical chemistry assay. In some aspects, the assay is a clinical chemistry assay and the sample is a whole blood, serum or plasma sample. In some aspects, the assay is a clinical chemistry assay and the sample is a whole blood sample. In yet other aspects, the assay is clinical chemistry assay and the sample is a plasma sample. In still yet other aspects, the assay is a clinical chemistry assay and the sample is a serum sample.

In some aspects, the assay is a single molecule detection assay. In some aspects, the assay is a single molecule detection assay and the sample is a whole blood, serum or plasma sample. In some aspects, the assay is a single molecule detection assay and the sample is a whole blood sample. In yet other aspects, the assay is single molecule detection assay and the sample is a plasma sample. In still yet other aspects, the assay is a single molecule detection assay and the sample is a serum sample.

In yet other embodiments, the present disclosure relates to methods of aiding in the diagnosis and evaluation of a human subject that has sustained or may have sustained an injury to the head. The methods comprise the steps of:
a) performing at least one assay on one or more samples taken from the subject to measure or detect a level of one or more biomarkers of TBI;
b) determining the amount of said one or more biomarkers in the one or more samples;
c) performing at least one advanced MRI procedure on the subject;
d) obtaining data from the procedure using at least one computer-implemented method;
e) generating a TBI diagnosis for the subject using the amount of the one or more biomarkers determined in step b) and the data obtained in step d); and

determining whether the subject has sustained a head injury or has not sustained a head injury based on the TBI diagnosis, wherein if the subject is determined to have sustained a head injury, determining (i) the pathophysiology of the head injury; (ii) the endotype of the head injury; or (iii) any combinations of (i)-(ii) endotype based on the TBI diagnosis.

In some aspects, the above methods further comprise whether the subject has, or more likely than not has, sustained a mild, moderate, severe, or moderate to severe TBI.

In the above methods, in some aspects, a first TBI diagnosis is generated for the subject based on the amount of the one or more biomarkers determined in step b). In some aspects, additionally, a second TBI diagnosis is generated based on the data obtained in step d). In some aspects, it is determined whether the subject has not sustained a head injury based on a combination of the first TBI diagnosis and the second TBI diagnosis.

In some aspects of the above methods, the advanced MRI procedure identifies the location of the TBI in the brain of the subject. In yet other aspects, the subject is predicted of having an unfavorable outcome based on the location of the TBI.

In some aspects, the pathophysiology of the head injury is determined by assessing whether the subject has or is suffering from at least one of cerebral hypofusion or hyperfusion, cerebral ischaemia, hyperaemia, impaired cerebrovascular CO₂-reactivity, a cerebral vasospasm, brain tissue hypoxia, oedema, oxidative stress, cell death or combinations thereof.

In some aspects, the endotype of the head injury is determined by determining (i) whether the injury is axonal, neuronal or astroglial; (ii) whether there is microvascular injury; (iii) whether there is proteinopathy; (iv) whether is there inflammation; or (v) any combinations thereof.

In some aspects, the above methods further comprise monitoring the subject. Alternatively, or in addition to, in some aspects, the above methods involve treating the subject.

In other aspects, the one or more biomarkers are selected from the group consisting of: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In some aspects, the one or more biomarkers comprises two or more biomarkers. In other aspects, the one or more biomarkers comprises three or more biomarkers. In yet other aspects, the one or more biomarkers comprises four or more biomarkers. In still yet other aspects, the one or more biomarkers comprises five or more biomarkers. In still yet other aspects, the one or more biomarkers comprises six or more biomarkers. In still yet other aspects, the one or more biomarkers comprises seven or more biomarkers. In still yet other aspects, the one or more biomarkers comprises eight or more biomarkers. In still yet other aspects, the one or more biomarkers comprises nine or more biomarkers. In still yet other aspects, the one or more biomarkers comprises ten or more biomarkers.

In yet other aspects, the one or more biomarkers are: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

In other aspects, the advanced MRI procedure is gradient echo magnetic resonance imaging, echo planar magnetic resonance imaging, spin echo magnetization resonance imaging, diffusion MRI, resting-state functional MRI, magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

In other aspects, the one or more samples is taken from the subject within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, with about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of the actual or suspected injury to the head.

In other aspects, the sample is selected from the group consisting of a whole blood sample, a serum sample, a cerebrospinal fluid sample, and a plasma sample. In some aspects, the sample is a whole blood sample. In other aspects, the sample is a serum sample. In yet other aspects, the sample is a cerebrospinal fluid sample. In still yet other aspects, the sample is a plasma sample. In still yet other aspects, the sample is a serum or plasma sample. In still yet other aspects, the sample is a whole blood, serum or plasma sample. In still other aspects, the sample is a whole blood or serum sample. In still yet other aspects, the sample is a whole blood or plasma sample.

In still yet other aspects, the sample is obtained after the subject sustained an injury to the head caused by physical shaking, blunt impact by an external mechanical or other force that results in a closed or open head trauma, one or more falls, explosions or blasts or other types of blunt force trauma. In yet other aspects, the sample is obtained after the subject has ingested or been exposed to a chemical, toxin or combination of a chemical and toxin. In some aspects, the chemical or toxin is fire, mold, asbestos, a pesticide, an insecticide, an organic solvent, a paint, a glue, a gas, an organic metal, a drug of abuse or one or more combinations thereof. In still yet other aspects, the sample is obtained from a subject that suffers from an autoimmune disease, a metabolic disorder, a brain tumor, hypoxia, a virus, meningitis, hydrocephalus or combinations thereof.

The above methods can be carried out on any subject without regard to factors selected from the group consisting of the subject's clinical condition, the subject's laboratory values, the subject's classification as suffering from mild, moderate, severe, or moderate to severe traumatic brain injury, the subject's exhibition of low or high levels of one or more biomarkers, and the timing of any event wherein said subject may have sustained an injury to the head.

In some aspects, the assay is an immunoassay. In some aspects, the assay is an immunoassay and the sample is a whole blood, serum or plasma sample. In some aspects, the assay is an immunoassay and the sample is a whole blood sample. In yet other aspects, the assay is an immunoassay and the sample is a plasma sample. In still yet other aspects, the assay is an immunoassay and the sample is a serum sample.

In some aspects, the assay is a clinical chemistry assay. In some aspects, the assay is a clinical chemistry assay and the sample is a whole blood, serum or plasma sample. In some aspects, the assay is a clinical chemistry assay and the sample is a whole blood sample. In yet other aspects, the assay is clinical chemistry assay and the sample is a plasma sample. In still yet other aspects, the assay is a clinical chemistry assay and the sample is a serum sample.

In some aspects, the assay is a single molecule detection assay. In some aspects, the assay is a single molecule detection assay and the sample is a whole blood, serum or plasma sample. In some aspects, the assay is a single molecule detection assay and the sample is a whole blood sample. In yet other aspects, the assay is single molecule detection assay and the sample is a plasma sample. In still yet other aspects, the assay is a single molecule detection assay and the sample is a serum sample.

In yet other embodiments, the present disclosure relates to methods for predicting or aiding in predicting the outcome of human subjects having a TBI. The methods comprise the steps of:
a) performing at least one assay on one or more samples taken from the subject to measure or detect a level of one or more biomarkers of TBI;
b) determining the amount of said one or more biomarkers in the one or more samples;
c) performing at least one advanced MRI procedure on the subject;
d) obtaining data from the procedure using at least one computer-implemented method;
e) generating a TBI outcome diagnosis for the subject using the amount of the one or more biomarkers determined in step b) and the data obtained in step d); and
f) predicting a favorable or unfavorable outcome in the subject based on the TBI outcome diagnosis.

In some aspects of the above methods, a first TBI outcome diagnosis is generated for the subject based on the amount of the one or more biomarkers determined in step b). In other aspects of the above methods, additionally, a second TBI outcome diagnosis is generated based on the data obtained in step d). In still yet other aspects, a favorable or unfavorable outcome is predicted for the subject based on a combination of the first TBI outcome diagnosis and the second TBI outcome diagnosis. In some aspects, the subject has a mild TBI. In other aspects, the subject has a moderate, severe, or moderate to severe TBI.

In some aspects, the above methods further comprise monitoring the subject. Alternatively, or in addition to, in some aspects, the above methods involve treating the subject.

In other aspects, the one or more biomarkers are selected from the group consisting of: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In some aspects, the one or more biomarkers comprises two or more biomarkers. In other aspects, the one or more biomarkers comprises three or more biomarkers. In yet other aspects, the one or more biomarkers comprises four or more biomarkers. In still yet other aspects, the one or more biomarkers comprises five or more biomarkers. In still yet other aspects, the one or more biomarkers comprises six or more biomarkers. In still yet other aspects, the one or more biomarkers comprises seven or more biomarkers. In still yet other aspects, the one or more biomarkers comprises eight or more biomarkers. In still yet other aspects, the one or more biomarkers comprises nine or more biomarkers. In still yet other aspects, the one or more biomarkers comprises ten or more biomarkers.

In yet other aspects, the one or more biomarkers are: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

In other aspects, the advanced MRI procedure is gradient echo magnetic resonance imaging, echo planar magnetic resonance imaging, spin echo magnetization resonance imaging, diffusion MRI, resting-state functional MRI, magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

In other aspects, the one or more samples is taken from the subject within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, with about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of the actual or suspected injury to the head.

In other aspects, the sample is selected from the group consisting of a whole blood sample, a serum sample, a cerebrospinal fluid sample, and a plasma sample. In some aspects, the sample is a whole blood sample. In other aspects, the sample is a serum sample. In yet other aspects, the sample is a cerebrospinal fluid sample. In still yet other aspects, the sample is a plasma sample. In still yet other aspects, the sample is a serum or plasma sample. In still yet other aspects, the sample is a whole blood, serum or plasma sample. In still other aspects, the sample is a whole blood or serum sample. In still yet other aspects, the sample is a whole blood or plasma sample.

In still yet other aspects, the sample is obtained after the subject sustained an injury to the head caused by physical shaking, blunt impact by an external mechanical or other force that results in a closed or open head trauma, one or more falls, explosions or blasts or other types of blunt force trauma. In yet other aspects, the sample is obtained after the subject has ingested or been exposed to a chemical, toxin or combination of a chemical and toxin. In some aspects, the chemical or toxin is fire, mold, asbestos, a pesticide, an insecticide, an organic solvent, a paint, a glue, a gas, an organic metal, a drug of abuse or one or more combinations thereof. In still yet other aspects, the sample is obtained from a subject that suffers from an autoimmune disease, a metabolic disorder, a brain tumor, hypoxia, a virus, meningitis, hydrocephalus or combinations thereof.

The above methods can be carried out on any subject without regard to factors selected from the group consisting of the subject's clinical condition, the subject's laboratory values, the subject's classification as suffering from mild, moderate, severe, or moderate to severe traumatic brain injury, the subject's exhibition of low or high levels of one or more biomarkers, and the timing of any event wherein said subject may have sustained an injury to the head.

In some aspects, the assay is an immunoassay. In some aspects, the assay is an immunoassay and the sample is a whole blood, serum or plasma sample. In some aspects, the assay is an immunoassay and the sample is a whole blood sample. In yet other aspects, the assay is an immunoassay and the sample is a plasma sample. In still yet other aspects, the assay is an immunoassay and the sample is a serum sample.

In some aspects, the assay is a clinical chemistry assay. In some aspects, the assay is a clinical chemistry assay and the sample is a whole blood, serum or plasma sample. In some aspects, the assay is a clinical chemistry assay and the sample is a whole blood sample. In yet other aspects, the assay is clinical chemistry assay and the sample is a plasma sample. In still yet other aspects, the assay is a clinical chemistry assay and the sample is a serum sample.

In some aspects, the assay is a single molecule detection assay. In some aspects, the assay is a single molecule detection assay and the sample is a whole blood, serum or plasma sample. In some aspects, the assay is a single molecule detection assay and the sample is a whole blood sample. In yet other aspects, the assay is single molecule detection assay and the sample is a plasma sample. In still yet other aspects, the assay is a single molecule detection assay and the sample is a serum sample.

In still yet other embodiments, the present disclosure relates to methods for predicting or aiding in predicting the outcome of human subjects having a traumatic brain injury. The methods comprise the steps of:
a) performing at least one assay on at least one or more samples taken from the subject to measure or detect a level of one or more biomarkers of TBI;
b) performing an advanced MRI procedure on the subject when the level of the one or more biomarkers in the one or more samples is unfavorable when compared to one or more reference levels of the one or more biomarkers; and
c) predicting an outcome in the human subject based on the results of the advanced MRI procedure.

In some aspects, the subject has a mild TBI. In other aspects, the subject has a moderate, severe, or moderate to severe TBI.

In some aspects, the above methods further comprise monitoring the subject. Alternatively, or in addition to, in some aspects, the above methods involve treating the subject.

In other aspects, the one or more biomarkers are selected from the group consisting of: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In some aspects, the one or more biomarkers comprises two or more biomarkers. In other aspects, the one or more biomarkers comprises three or more biomarkers. In yet other aspects, the one or more biomarkers comprises four or more biomarkers. In still yet other aspects, the one or more biomarkers comprises five or more biomarkers. In still yet other aspects, the one or more biomarkers comprises six or more biomarkers. In still yet other aspects, the one or more biomarkers comprises seven or more biomarkers. In still yet other aspects, the one or more biomarkers comprises eight or more biomarkers. In still yet other aspects, the one or more biomarkers comprises nine or more biomarkers. In still yet other aspects, the one or more biomarkers comprises ten or more biomarkers.

In yet other aspects, the one or more biomarkers are: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

In other aspects, the advanced MRI procedure is gradient echo magnetic resonance imaging, echo planar magnetic resonance imaging, spin echo magnetization resonance imaging, diffusion MRI, resting-state functional MRI, magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

In other aspects, the one or more samples is taken from the subject within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, with about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of the actual or suspected injury to the head.

In other aspects, the sample is selected from the group consisting of a whole blood sample, a serum sample, a cerebrospinal fluid sample, and a plasma sample. In some aspects, the sample is a whole blood sample. In other aspects, the sample is a serum sample. In yet other aspects, the sample is a cerebrospinal fluid sample. In still yet other aspects, the sample is a plasma sample. In still yet other aspects, the sample is a serum or plasma sample. In still yet other aspects, the sample is a whole blood, serum or plasma sample. In still other aspects, the sample is a whole blood or serum sample. In still yet other aspects, the sample is a whole blood or plasma sample.

In still yet other aspects, the sample is obtained after the subject sustained an injury to the head caused by physical shaking, blunt impact by an external mechanical or other force that results in a closed or open head trauma, one or more falls, explosions or blasts or other types of blunt force trauma. In yet other aspects, the sample is obtained after the subject has ingested or been exposed to a chemical, toxin or combination of a chemical and toxin. In some aspects, the chemical or toxin is fire, mold, asbestos, a pesticide, an insecticide, an organic solvent, a paint, a glue, a gas, an organic metal, a drug of abuse or one or more combinations thereof. In still yet other aspects, the sample is obtained from a subject that suffers from an autoimmune disease, a metabolic disorder, a brain tumor, hypoxia, a virus, meningitis, hydrocephalus or combinations thereof.

The above methods can be carried out on any subject without regard to factors selected from the group consisting of the subject's clinical condition, the subject's laboratory values, the subject's classification as suffering from mild, moderate, severe, or moderate to severe traumatic brain injury, the subject's exhibition of low or high levels of one or more biomarkers, and the timing of any event wherein said subject may have sustained an injury to the head.

In some aspects, the assay is an immunoassay. In some aspects, the assay is an immunoassay and the sample is a whole blood, serum or plasma sample. In some aspects, the assay is an immunoassay and the sample is a whole blood sample. In yet other aspects, the assay is an immunoassay and the sample is a plasma sample. In still yet other aspects, the assay is an immunoassay and the sample is a serum sample.

In some aspects, the assay is a clinical chemistry assay. In some aspects, the assay is a clinical chemistry assay and the sample is a whole blood, serum or plasma sample. In some aspects, the assay is a clinical chemistry assay and the sample is a whole blood sample. In yet other aspects, the assay is clinical chemistry assay and the sample is a plasma sample. In still yet other aspects, the assay is a clinical chemistry assay and the sample is a serum sample.

In some aspects, the assay is a single molecule detection assay. In some aspects, the assay is a single molecule detection assay and the sample is a whole blood, serum or plasma sample. In some aspects, the assay is a single molecule detection assay and the sample is a whole blood sample. In yet other aspects, the assay is single molecule detection assay and the sample is a plasma sample. In still yet other aspects, the assay is a single molecule detection assay and the sample is a serum sample.

In still yet other embodiments, the present disclosure relates to methods for determining a course of treatment or efficacy of a course of treatment for a human subject who has suffered a TBI. The methods comprise the steps of:
a. performing at least one assay on one or more samples taken from the subject to measure or detect a level of one or more biomarkers of TBI;
b. determining the amount of said one or more biomarkers in the one or more samples;
c. performing at least one advanced MRI procedure on the subject after diagnosis of a traumatic brain injury;
d. obtaining data from the at least one advanced MRI procedure performed in step c) using a computer-implemented method;
e. generating a TBI treatment protocol for the subject using the amount of the one or more biomarkers in the one or more samples obtained in step b) and the data obtained in step d) for assessing head injury; and
f. determine a course of treatment or the efficacy of a course of treatment in the subject based on the TBI treatment protocol.

In some aspects of the above methods, a first TBI treatment protocol is generated for the subject based on the amount of the one or more biomarkers determined in step b). In other aspects of the above methods, additionally, a second TBI treatment protocol is generated based on the data obtained in step d). In still yet other aspects of the above methods, the course of treatment or the efficacy of the course of treatment is determined based on a combination of the first TBI treatment protocol and the second TBI treatment protocol. Moreover, in other aspects of the above methods, the subject has been receiving treatment for TBI. In other aspects, the subject has not been receiving treatment for TBI.

In some aspects, the subject has a mild TBI. In other aspects, the subject has a moderate, severe, or moderate to severe TBI.

In some aspects, the above methods further comprise monitoring the subject. Alternatively, or in addition to, in some aspects, the above methods involve treating the subject.

In other aspects, the one or more biomarkers are selected from the group consisting of: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In some aspects, the one or more biomarkers comprises two or more biomarkers. In other aspects, the one or more biomarkers comprises three or more biomarkers. In yet other aspects, the one or more biomarkers comprises four or more biomarkers. In still yet other aspects, the one or more biomarkers comprises five or more biomarkers. In still yet other aspects, the one or more biomarkers comprises six or more biomarkers. In still yet other aspects, the one or more biomarkers comprises seven or more biomarkers. In still yet other aspects, the one or more biomarkers comprises eight or more biomarkers. In still yet other aspects, the one or more biomarkers comprises nine or more biomarkers. In still yet other aspects, the one or more biomarkers comprises ten or more biomarkers.

In yet other aspects, the one or more biomarkers are: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

In other aspects, the advanced MRI procedure is gradient echo magnetic resonance imaging, echo planar magnetic resonance imaging, spin echo magnetization resonance imaging, diffusion MRI (e.g, multi-shell diffusion MRI), resting-state functional MRI (fMRI), magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

In other aspects, the one or more samples is taken from the subject within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, with about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of the actual or suspected injury to the head.

In other aspects, the sample is selected from the group consisting of a whole blood sample, a serum sample, a cerebrospinal fluid sample, and a plasma sample. In some aspects, the sample is a whole blood sample. In other aspects, the sample is a serum sample. In yet other aspects, the sample is a cerebrospinal fluid sample. In still yet other aspects, the sample is a plasma sample. In still yet other aspects, the sample is a serum or plasma sample. In still yet other aspects, the sample is a whole blood, serum or plasma sample. In still other aspects, the sample is a whole blood or serum sample. In still yet other aspects, the sample is a whole blood or plasma sample.

In still yet other aspects, the sample is obtained after the subject sustained an injury to the head caused by physical shaking, blunt impact by an external mechanical or other force that results in a closed or open head trauma, one or more falls, explosions or blasts or other types of blunt force trauma. In yet other aspects, the sample is obtained after the subject has ingested or been exposed to a chemical, toxin or combination of a chemical and toxin. In some aspects, the chemical or toxin is fire, mold, asbestos, a pesticide, an insecticide, an organic solvent, a paint, a glue, a gas, an organic metal, a drug of abuse or one or more combinations thereof. In still yet other aspects, the sample is obtained from a subject that suffers from an autoimmune disease, a metabolic disorder, a brain tumor, hypoxia, a virus, meningitis, hydrocephalus or combinations thereof.

The above methods can be carried out on any subject without regard to factors selected from the group consisting of the subject's clinical condition, the subject's laboratory values, the subject's classification as suffering from mild, moderate, severe, or moderate to severe traumatic brain injury, the subject's exhibition of low or high levels of one or more biomarkers, and the timing of any event wherein said subject may have sustained an injury to the head.

In some aspects, the assay is an immunoassay. In some aspects, the assay is an immunoassay and the sample is a whole blood, serum or plasma sample. In some aspects, the assay is an immunoassay and the sample is a whole blood sample. In yet other aspects, the assay is an immunoassay and the sample is a plasma sample. In still yet other aspects, the assay is an immunoassay and the sample is a serum sample.

In some aspects, the assay is a clinical chemistry assay. In some aspects, the assay is a clinical chemistry assay and the sample is a whole blood, serum or plasma sample. In some aspects, the assay is a clinical chemistry assay and the sample is a whole blood sample. In yet other aspects, the assay is clinical chemistry assay and the sample is a plasma sample. In still yet other aspects, the assay is a clinical chemistry assay and the sample is a serum sample.

In some aspects, the assay is a single molecule detection assay. In some aspects, the assay is a single molecule detection assay and the sample is a whole blood, serum or plasma sample. In some aspects, the assay is a single molecule detection assay and the sample is a whole blood sample. In yet other aspects, the assay is single molecule detection assay and the sample is a plasma sample. In still yet other aspects, the assay is a single molecule detection assay and the sample is a serum sample.

### DETAILED DESCRIPTION

In one aspect, the present disclosure relates to methods of determining or evaluating a subject to determine whether to perform an advanced magnetic resonance imaging (MRI) procedure on a subject having a head injury or suspected of having a head injury. The methods involve performing at least one assay on one or more samples obtained from a subject after an injury or suspected injury to the head to measure or detect in the one or more samples a level of one or more biomarkers of traumatic brain injury (TBI) and then determining whether to perform an advanced MRI procedure on the subject when the level of one or more biomarkers in the one or more samples is unfavorable when compared to one or more reference levels and not performing an advanced MRI procedure on the subject when the level of the one or more biomarkers in the one or more samples is favorable than one or more reference levels.

In the above described methods, the subject may have received an imaging procedure (such as a conventional MRI procedure or an advanced MRI procedure) before or after the assay is performed. In some aspects, the subject may be suspected of or identified as having a TBI based on the MRI procedure conducted prior to the assay being performed. In some aspects, the reference level is correlated with a positive MRI. In other aspects, the reference level is correlated with the presence of an intracranial lesion. In other aspects, the reference level is correlated with control subjects that have not sustained a head injury.

The methods described above involve performing at least one assay on one or more samples taken from the subject within 24 hours, within 48 hours, within 72 hours, etc. after an injury or suspected head injury to measure or detect a level of one or more biomarkers. The one or more biomarkers to be measured or detected can be: a) one or more biomarkers of neuronal or axonal injury, one or more biomarkers of astroglial injury, one or more biomarkers of neuroinflammation, one or more genetic biomarkers, and any combinations thereof; b) one or more autoantibodies of one or more biomarkers of neuronal or axonal injury, one or more biomarkers of astroglial injury, one or more biomarkers of neuroinflammation, one or more genetic biomarkers, and any combinations thereof; and c) any combinations of one or more biomarkers of neuronal or axonal injury, one or more biomarkers of astroglial injury, one or more biomarkers of neuroinflammation, one or more genetic biomarkers, and any combinations thereof and one or more autoantibodies of biomarkers of neuronal or axonal injury, biomarkers of astroglial injury, biomarkers of neuroinflammation, genetic biomarkers. In another aspect, one or more biomarkers that can be used in the methods described herein include: a) APOA1, CRP, NSE, BDNF, tau, p-tau, NF-L, S100β, UCH-L1, GFAP and combinations thereof; b) one or more autoantibodies to any of APOA1, CRP, NSE, BDNF, tau, p-tau, NF-L, S100β, UCH-L1, GFAP and combinations thereof; or c) any combination of one or more biomarkers of APOA1, CRP, NSE, BDNF, tau, p-tau, NF-L, S100β, UCH-L1, GFAP and one or more autoantibodies to APOA1, CRP, NSE, BDNF, tau, p-tau, NF-L, S100β, UCH-L1, GFAP.

In another aspect, the present disclosure relates to methods of evaluating or aiding in the diagnosis and evaluation of whether a human subject has sustained or may have sustained an injury to the head and, if so, whether the subject has sustained a TBI. For example, the methods described herein can aid in determining that a subject suspected of a head injury (e.g., injury to the head) has in fact, not suffered a head injury. Alternatively, the methods can aid in determining that a subject with a suspected head injury has in fact suffered a head injury, but not a TBI. Still further, the methods can aid in determining that a subject with a suspected head injury has in fact suffered head injury, specifically, a TBI. Yet further, the methods can aid in determining the extent of TBI in a human subject, e.g., determining that the subject has a mild traumatic brain injury, moderate traumatic brain injury, severe traumatic brain injury, or a moderate to severe traumatic brain injury. The methods involve performing at least one assay on a sample taken from the subject within 24 hours, within 48 hours, within 72 hours, etc. after a suspected head injury to measure or detect a level of one or more biomarkers. The one or more biomarkers can be: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In another aspect, one or more biomarkers that can be used in the methods described herein include: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

After the at least one assay is performed, the amount of one or more biomarkers in the one or more samples is determined. In one aspect, a first TBI diagnosis for the subject is generated based on the amount of the one or more biomarkers determined by the assay. Alternatively, the amount of the one or more biomarkers determined (e.g., measured) by the at least one assay is used in a computer-implemented method to generate a single TBI diagnosis as discussed in more detail herein.

Either before or after the at least one assays are performed, at least one advanced MRI procedure is performed on the subject. The advanced MRI procedure can include, for example, gradient echo magnetic resonance imaging, echo planar magnetic resonance imaging, spin echo magnetization resonance imaging, diffusion MRI (e.g., such as multi-shell diffusion MRI), resting-state functional MRI (fMRI), magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof. In one aspect, the data obtained from at least one advanced magnetic resonance imaging procedure is used in a computer-implemented method for assessing head injury to generate a second TBI diagnosis for the subject.

Once the first and second TBI diagnosis are generated, these diagnoses are combined to generate a final TBI diagnosis. In one aspect, the final TBI diagnosis is that the subject has not sustained a head injury. In another aspect, the final TBI diagnosis is that the subject has sustained a head injury. For subjects identified or determined to have sustained a head injury, the final TBI diagnosis can be used to assess the (i) pathophysiology of the head injury, such as, for example, to identify or characterize that the subject has, is suffering or is at risk of suffering from one or more of cerebral hypofusion or hyperfusion, cerebral ischaemia, hyperaemia, impaired cerebrovascular CO₂-reactivity (such as, for example, cerebrovascular constriction or dilation in response to hypo- or hypercapnia), a cerebral vasospasm, brain tissue hypoxia, oedema (such as vasogenic brain or cytotoxic brain oedema), oxidative stress, cell death (such as, for example, necrosis or apoptosis) or any combinations thereof; (ii) the endotype of the head injury, such as, for example, determining whether the injury is axonal, neuronal or astroglial, whether there is microvascular injury, whether there is proteinopathy, and/or whether there is inflammation, or any combinations thereof; (iii) whether the subject has, or more likely than not has, sustained a mild, moderate, severe, or moderate to severe TBI; or (iv) any combinations of (i)-(iii). Information on the pathophysiology of the head injury, the endotype of the head injury and/or type of TBI (e.g., mild, moderate, severe, or moderate to severe TBI) determined using the methods described herein can be used to guide and manage treatment decisions and options for the subject, monitoring of the subject and/or predict the outcome of the subject.

Alternatively, in another aspect, the amount of the one or more biomarkers assessed by the assay and the data obtained from the at least one advanced MRI procedure can be used in a computer-implemented method to generate a single TBI diagnosis of whether the subject has sustained no head injury or that the subject has sustained a head injury. For subjects identified or determined to have sustained a head injury, the single TBI diagnosis can be used to assess the (i) pathophysiology of the head injury, such as, for example, to identify or characterize that the subject has, is suffering or is at risk of suffering from one or more of cerebral hypofusion or hyperfusion, cerebral ischaemia, hyperaemia, impaired cerebrovascular CO₂-reactivity (such as, for example, cerebrovascular constriction or dilation in response to hypo- or hypercapnia), a cerebral vasospasm, brain tissue hypoxia, oedema (such as vasogenic brain or cytotoxic brain oedema), oxidative stress, cell death (such as, for example, necrosis or apoptosis) or any combinations thereof; (ii) the endotype of the head injury, such as, for example, determining whether the injury is axonal, neuronal or astroglial, whether there is microvascular injury, whether there is proteinopathy, is there inflammation or any combinations thereof; (iii) whether the subject has, or more likely than not has, sustained a mild, moderate, severe, or moderate to severe TBI; or (iv) any combinations of (i)-(iii). Information on the pathophysiology of the head injury, the endotype of the head injury and/or type of TBI (e.g., mild, moderate, severe, or moderate to severe TBI) assessed using the methods described herein can be used to guide and manage treatment decisions and options for the subject, monitoring of the subject and/or predict the outcome of the subject.

In yet another aspect, the present disclosure also relates to methods of predicting the outcome of human subjects that have suffered a traumatic brain injury (such as a mild, moderate, severe, or moderate to severe TBI). The methods involve performing at least one assay on one or more samples taken from the subject (such as, for example, within 24 hours, within 48 hours, within 72 hours, etc.) after diagnosis of a TBI to measure or detect a level of one or more biomarkers. The one or more biomarkers can be: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In another aspect, one or more biomarkers that can be used in the methods described herein include: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

The at least one assay that is to be performed allows the amount of one or more biomarkers in the one or more samples to be determined. An advanced MRI procedure is performed on the subject if the level of the one or more biomarkers in the one or more samples is higher than one or more reference levels of the one or more biomarkers. The outcome in the human subject can be predicted based on the results of the advanced MRI procedure. For example, depending on the location of the TBI, the outcome may be favorable or unfavorable. Similarly, the course of the injury (i.e., disability) and recovery can be predicted, e.g., if cognitive, emotional, psychological or motor skills are primarily impacted.

In still yet another aspect, the present disclosure also relates to methods of predicting the outcome of human subjects that have suffered a traumatic brain injury (such as a mild, moderate, severe, or moderate to severe TBI). The methods involve performing at least one assay on one or more samples taken from the subject after diagnosis of a TBI to measure or detect a level of one or more biomarkers. The one or more biomarkers can be: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In another aspect, one or more biomarkers that can be used in the methods described herein include: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

A first TBI outcome diagnosis for the subject is generated based on the amount of the one or more biomarkers determined by the assay. Alternatively, the amount of the one or more biomarkers determined (e.g., measured) by the at least one assay is used in a computer-implemented method to generate a single TBI outcome diagnosis as discussed in more detail herein.

Either before or after the at least one assays are performed, at least one advanced MRI procedure is performed on the subject (such as, for example, within 24 hours, within 48 hours, within 72 hours, etc.) after the suspected head injury. The advanced MRI procedure can include, for example, gradient echo magnetic resonance imaging, echo planar magnetic resonance imaging, spin echo magnetization resonance imaging, diffusion MRI (e.g, such as multi-shell diffusion MRI), resting-state functional MRI (fMRI), magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof. In one aspect, the data obtained from at least one advanced MRI procedure is used in a computer-implemented method for assessing head injury to generate a second TBI outcome diagnosis for the subject.

Once the first and second TBI diagnosis are generated, these diagnoses are combined to generate a final TBI outcome diagnosis of whether the subject will likely have a favorable outcome as a result of the TBI or will not likely have a favorable outcome as a result of the TBI. In one aspect, the favorable or unfavorable outcome can be based on the pathophysiology of the head injury, the endotype of the head injury and/or the type of TBI (e.g., mild, moderate, severe, or moderate to severe TBI) assessed as described herein.

Alternatively, in another aspect, the amount of the one or more biomarkers determined (i.e., measured) by the assay and the data obtained from the at least one advanced MRI procedure can be used in a computer-implemented method to generate a single TBI outcome diagnosis of whether the subject will likely have a favorable outcome as a result of the TBI or will not likely have a favorable outcome as a result of the TBI.

The present disclosure also relates to methods of determining a course of treatment or determining the efficacy of a course of treatment in a human subject that has suffered a traumatic brain injury. In one aspect, determining a course of treatment or efficacy of a course of treatment in a subject can be based on the determination of the pathophysiology, the endotype of the head injury and/or type of TBI (e.g., mild, moderate, severe, or moderate to severe TBI) as described herein. The methods involve performing at least one assay on one or more samples taken from the subject diagnosed with a TBI to measure or detect a level of one or more biomarkers. The one or more biomarkers can be: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In another aspect, one or more biomarkers that can be used in the methods described herein include: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b). After the at least one assay is performed, the amount of one or more biomarkers in the one or more samples is determined. In one aspect, a first TBI treatment protocol for the subject is generated based on the amount of the one or more biomarkers determined (e.g., measured) by the assay. Alternatively, the amount of the one or more biomarkers determined (e.g., measured) by the at least one assay is used in a computer-implemented method to generate a single TBI treatment protocol as discussed in more detail herein.

Either before or after the at least one assays are performed, at least one advanced MRI procedure is performed on the subject (such as, for example, within 24 hours, within 48 hours, within 72 hours, etc.) after the suspected head injury. The advanced MRI procedure can be: gradient echo magnetic resonance imaging, echo planar magnetic resonance imaging, spin echo magnetization resonance imaging, diffusion MRI (e.g., multi-shell diffusion MRI), resting-state functional MRI (fMRI), magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

Once the first and second TBI diagnosis are generated, these diagnoses are combined to generate a final TBI treatment protocol of whether the subject is in need of a course of treatment (e.g., such as in circumstances where the subject was not previously receiving any treatment), or whether an existing course of treatment is efficacious for the subject or whether a course of treatment for the subject needs to be altered, discontinued or supplemented. Alternatively, in another aspect, the amount of the one or more biomarkers determined by the assay and the data obtained from the at least one advanced MRI procedure can be used in a computer-implemented method to generate a single TBI treatment protocol of whether the subject will likely have a favorable outcome as a result of the TBI or will not likely have a favorable outcome as a result of the TBI.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Advanced magnetic resonance imaging procedure(s)” or “advanced MRI procedure(s)” as used interchangeably, refers to a form of medical imaging that measures the response of the atomic nuclei of body tissues to high-frequency radio waves when placed in a strong magnetic field, and that produces images of the internal organs. MRI scanners, which are based on the science of nuclear magnetic resonance (NMR), use strong magnetic fields, radio waves, and field gradients to generate images of the inside of the body. Unlike conventional or standard MRI procedures, advanced MRI procedures can generate physiologic and microstructure data as well as information on chemical composition of the organ, soft tissue, bone or other internal body structure being analyzed. Examples of advanced MRI procedures include gradient echo magnetic resonance imaging, echo planar magnetic resonance imaging, spin echo magnetization resonance imaging, diffusion MRI (e.g, multi-shell diffusion MRI), resting-state functional MRI (fMRI), magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

“Affinity matured antibody” is used herein to refer to an antibody with one or more alterations in one or more CDRs, which result in an improvement in the affinity (e.g., K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies is known in the art, including the screening of a combinatory antibody library that has been prepared using bio-display. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); and Hawkins et al., J. Mol. Biol., 226: 889-896 (1992). Selective mutation at selective mutagenesis positions and at contact or hypermutation positions with an activity-enhancing amino acid residue is described in U.S. Pat. No. 6,914,128 B1.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody” (e.g., an anti-NF-L antibody, anti-NSE antibody, anti-S100β antibody, an anti-GFAP antibody, an anti-UCH-L1 antibody, etc.).

“Antibody fragment” as used herein refers to a portion of an intact antibody comprising the antigen-binding site or variable region. The portion does not include the constant heavy chain domains (e.g., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

The “area under curve” or “AUC” refers to area under a ROC curve. AUC under a ROC curve is a measure of accuracy. An AUC of 1 represents a perfect test, whereas an AUC of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

“Bead” and “particle” are used herein interchangeably and refer to a substantially spherical solid support. One example of a bead or particle is a microparticle. Microparticles that can be used herein can be any type known in the art. For example, the bead or particle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO.Fe₂O₃). Beads can have a solid core portion that is magnetic and is surrounded by one or more non-magnetic layers. Alternately, the magnetic portion can be a layer around a non-magnetic core. The microparticles can be of any size that would work in the methods described herein, e.g., from about 0.75 to about 5 nm, or from about 1 to about 5 nm, or from about 1 to about 3 nm.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Biomarker” is used herein to refer to a cell, a nucleic acid, a protein (including antibodies), a metabolite, or combinations thereof that is differentially present (namely, increased or decreased) in a biological sample from a subject or a group of subjects having a first phenotype (e.g., having a disease or condition) as compared to a biological sample from a subject or group of subjects having a second phenotype (e.g., not having the disease or condition or having a less severe version of the disease or condition). In one aspect, the biomarker is a “brain biomarker”. A “brain biomarker” refers to one or more biomarkers that can be used to evaluate or aid in the diagnosis and evaluation of a human subject for a head injury (such as a traumatic brain injury), predict the outcome of human subjects having a traumatic brain injury or determine the course of treatment or efficacy of a course of treatment for a human subject who has suffered a brain injury. Additionally and alternatively, brain biomarkers can be used to provide a measure or indication of TBI. Examples of brain biomarkers include a) one or more biomarkers of neuronal or axonal injury, one or more biomarkers of astroglial injury, one or more biomarkers of neuroinflammation, one or more genetic biomarkers, or any combinations thereof; b) one or more autoantibodies to one or more biomarkers of neuronal or axonal injury, one or more biomarkers of astroglial injury, one or more biomarkers of neuroinflammation, one or more genetic biomarkers or combinations thereof; or c) any combinations of one or more biomarkers of neuronal or axonal injury, one or more biomarkers of astroglial injury, one or more biomarkers of neuroinflammation, one or more genetic biomarkers, and one or more autoantibodies to one or more biomarkers of neuronal or axonal injury, one or more biomarkers of astroglial injury, one or more biomarkers of neuroinflammation, or one or more genetic biomarkers. Examples of one or more biomarkers of neuronal or axonal injury include: Tau, p-Tau, ubiquitin carboxyl-terminal hydrolase isoenzyme L1 (UCH-L1), neurofilaments (such as neurofilament light polypeptide (NF-L) and phosphorylated versions thereof), neuron specific enolase (NSE), brain derived nerve growth factor (BDNF; intact and free), and combinations thereof. Examples of one or more biomarkers of astroglial injury include: calcium-binding protein (S100β), glial fibrillary acidic protein (GFAP), glial fibrillary acidic protein breakdown products (GFAP-BDP), perooxiredoxin 6 (PRDX6), and any combinations thereof. Examples of one or more biomarkers of neuroinflammation include: tumor necrosis factor (TNF), interleukin (IL) alphas and betas (such as IL-6, IL-10, etc.), IGF1, highly sensitive c reactive protein (CRP), apolipoprotein (such as APOA (such as APOA1 and negative acute phase marker), and combinations thereof. Examples of one or more genetic biomarkers include microRNA (miRNA).

In another aspect, one or more biomarkers that can be used in the methods described herein include APOA1, CRP, NSE, BDNF, tau, p-tau, NF-L, S100β, UCH-L1, GFAP and combinations thereof as well as one or more autoantibodies to any of these biomarkers. In yet another aspect, one or more biomarkers that can be used in the methods described herein include APOA1, CRP, NSE, BDNF, tau, p-tau, NF-L, S100β, UCH-L1, GFAP and combinations thereof, autoantibodies of one or more of APOA1, CRP, NSE, BDNF, tau, p-tau, NF-L, S100β, UCH-L1, GFAP and combinations thereof or any combinations of APOA1, CRP, NSE, BDNF, tau, p-tau, NF-L, S100β, UCH-L1, GFAP and one or more autoantibodies to APOA1, CRP, NSE, BDNF, tau, p-tau, NF-L, S100β, UCH-L1, GFAP.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds to.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9: 133-139 (1995), and MacCallum, J. Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, whole blood, serum or plasma sample, in accordance with the methods described herein and other methods known in the art. Some components can be in solution or lyophilized for reconstitution for use in an assay.

“Conventional or standard magnetic resonance imaging procedure(s)” or “conventional or standard MRI imaging procedure(s)” as used interchangeably herein refers to a medical imaging procedure used in radiology to form pictures of the anatomy and the physiological processes of the body in both health and disease (e.g., referred to herein interchangeably as “an MRI”, “an MRI procedure” or “an MRI scan”). MRI is a form of medical imaging that measures the response of the atomic nuclei of body tissues to high-frequency radio waves when placed in a strong magnetic field, and that produces images of the internal organs. MRI scanners, which is based on the science of nuclear magnetic resonance (NMR), use strong magnetic fields, radio waves, and field gradients to generate images of the inside of the body. The images provided by conventional or standard MRI imaging procedures do not reflect the gross anatomy of the structure being examined and as such, are unable to generate physiologic and microscopic data as well as information on the chemical composition of that structure. As a result, a conventional or standard MRI imaging procedure cannot detect the presence of different injury sub-types or group subjects based on pathology.

“Correlated to” as used herein refers to compared to.

“CT scan” as used herein refers to a computerized tomography (CT) scan. A CT scan combines a series of X-ray images taken from different angles and uses computer processing to create cross-sectional images, or slices, of the bones, blood vessels and soft tissues inside your body. The CT scan may use X-ray CT, positron emission tomography (PET), single-photon emission computed tomography (SPECT), computed axial tomography (CAT scan), or computer aided tomography. The CT scan may be a conventional CT scan or a spiral/helical CT scan. In a conventional CT scan, the scan is taken slice by slice and after each slice the scan stops and moves down to the next slice, e.g., from the top of the abdomen down to the pelvis. The conventional CT scan requires patients to hold their breath to avoid movement artefact. The spiral/helical CT scan is a continuous scan which is taken in a spiral fashion and is a much quicker process where the scanned images are contiguous.

“Determined by an assay” is used herein to refer to the determination (e.g., measurement) of a reference level by any appropriate assay. The determination of a reference level may, in some embodiments, be achieved by an assay of the same type as the assay that is to be applied to the sample from the subject (for example, by an immunoassay, clinical chemistry assay, a single molecule detection assay, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS)). The determination of a reference level may, in some embodiments, be achieved by an assay of the same type and under the same assay conditions as the assay that is to be applied to the sample from the subject. As noted herein, this disclosure provides exemplary reference levels (e.g., calculated by comparing reference levels at different time points). It is well within the ordinary skill of one in the art to adapt the disclosure herein for other assays to obtain assay-specific reference levels for those other assays based on the description provided by this disclosure. For example, a set of training samples comprising samples obtained from human subjects known to have sustained an injury to the head (and more particularly, samples obtained from human subjects known to have sustained a (i) mild TBI; and/or (ii) moderate, severe, or moderate to severe TBI and samples obtained from human subjects known not to have sustained an injury to the head may be used to obtain assay-specific reference levels. It will be understood that a reference level “determined by an assay” and having a recited level of “sensitivity” and/or “specificity” is used herein to refer to a reference level which has been determined to provide a method of the recited sensitivity and/or specificity when said reference level is adopted in the methods of the invention. It is well within the ordinary skill of one in the art to determine the sensitivity and specificity associated with a given reference level in the methods of the invention, for example by repeated statistical analysis of assay data using a plurality of different possible reference levels.

Practically, when discriminating between a subject as having a traumatic brain injury or not having a traumatic brain injury or a subject as having a mild versus a moderate, severe, or moderate to severe traumatic brain injury, the skilled person will balance the effect of raising a cutoff on sensitivity and specificity. Raising or lowering a cutoff will have a well-defined and predictable impact on sensitivity and specificity, and other standard statistical measures. It is well known that raising a cutoff will improve specificity but is likely to worsen sensitivity (proportion of those with disease who test positive). In contrast, lowering a cutoff will improve sensitivity but will worsen specificity (proportion of those without disease who test negative). The ramifications for detecting traumatic brain injury or determining a mild versus moderate, severe, or moderate to severe traumatic brain injury will be readily apparent to those skilled in the art. In discriminating whether a subject has or does not have a traumatic brain injury or a mild versus a moderate, severe, or moderate to severe traumatic brain injury, the higher the cutoff, specificity improves as more true negatives (e.g., subjects not having a traumatic brain injury, not having a mild traumatic brain injury, not have a moderate traumatic brain injury, not having a severe traumatic brain injury or not having a moderate to severe traumatic brain injury) are distinguished from those having a traumatic brain injury, a mild traumatic brain injury, a moderate traumatic brain injury, a severe traumatic brain injury or a moderate to severe traumatic brain injury. But at the same time, raising the cutoff decreases the number of cases identified as positive overall, as well as the number of true positives, so the sensitivity must decrease. Conversely, the lower the cutoff, sensitivity improves as more true positives (e.g., subjects having a traumatic brain injury, having a mild traumatic brain injury, having a moderate traumatic brain injury, having a severe traumatic brain injury or having a moderate to severe traumatic brain injury) are distinguished from those who do not have a traumatic brain injury, a mild traumatic brain injury, a moderate traumatic brain injury, a severe traumatic brain injury or a moderate to severe traumatic brain injury. But at the same time, lowering the cutoff increases the number of cases identified as positive overall, as well as the number of false positives, so the specificity must decrease.

Generally, a high sensitivity value helps one of skill rule out disease or condition (such as a traumatic brain injury, mild traumatic brain injury, moderate traumatic brain injury, severe traumatic brain injury or moderate to severe traumatic brain injury), and a high specificity value helps one of skill rule in disease or condition. Whether one of skill desires to rule out or rule in disease depends on what the consequences are for the patient for each type of error. Accordingly, one cannot know or predict the precise balancing employed to derive a test cutoff without full disclosure of the underlying information on how the value was selected. The balancing of sensitivity against specificity and other factors will differ on a case-by-case basis. This is why it is sometimes preferable to provide alternate cutoff (e.g., reference) values so a physician or practitioner can choose.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Drugs of abuse” is used herein to refer to one or more additive substances (such as a drug) taken for non-medical reasons (such as for, example, recreational and/or mind-altering effects). Excessive overindulgence, use or dependence of such drugs of abuse is often referred to as “substance abuse”. Examples of drugs of abuse include alcohol, barbiturates, benzodiazepines, cannabis, cocaine, hallucinogens (such as ketamine, mescaline (peyote), PCP, psilocybin, DMT and/or LSD), methaqualone, opioids, amphetamines (including methamphetamines), anabolic steroids, inhalants (namely, substances which contain volatile substances that contain psychoactive properties such as, for example, nitrites, spray paints, cleaning fluids, markers, glues, etc.) and combinations thereof.

“Dual-specific antibody” is used herein to refer to a full-length antibody that can bind two different antigens (or epitopes) in each of its two binding arms (a pair of HC/LC) (see PCT publication WO 02/02773). Accordingly, a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, e.g., capable of binding one antigen (or one specific epitope), or multispecific, e.g., capable of binding two or more antigens (e.g., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule, but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (e.g., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25: 1290-1297 (2007). A preferred example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In a preferred embodiment, a DVD-Ig binding protein not only binds the same target molecules bound by its parental monoclonal antibodies, but also possesses one or more desirable properties of one or more of its parental monoclonal antibodies. Preferably, such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

A DVD-Ig binding protein binds at least one epitope of a biomarker (e.g., APOA1, CRP, NSE, BDNF, tau, p-tau, NF-L, S100β, UCH-L1, GFAP, etc.) Non-limiting examples of a DVD-Ig binding protein include a DVD-Ig binding protein that binds one or more epitopes of a biomarker (e.g., APOA1, CRP, NSE, BDNF, tau, p-tau, NF-L, S100β, UCH-L1, GFAP), a DVD-Ig binding protein that binds an epitope of a human biomarker and an epitope of biomarker of another species (for example, mouse), and a DVD-Ig binding protein that binds an epitope of a human biomarker and an epitope of another target molecule.

“Dynamic range” as used herein refers to range over which an assay readout is proportional to the amount of target molecule or analyte in the sample being analyzed.

As used herein, an “endotype” refers to subtype of injury. (An endotype is a subtype of a condition, which is defined by a distinct functional or pathobiological mechanism. This is distinct from a phenotype, which is any observable characteristic or trait of a disease, such as morphology, development, biochemical or physiological properties, or behavior, without any implication of a mechanism). Endotype is related to, and yet differs from, the pathophysiology of a disease disorder or condition. “Pathophysiology” as used herein refers to the physiology of abnormal states, and specifically, the functional changes that accompany a particular disease, disorder condition. Examples of endotypes related to TBI as described herein include, but are not limited to, the identification of whether a head injury is axonal, neuronal or astroglial, whether there is microvascular injury, whether there is proteinopathy, whether there is inflammation, whether biomarker levels are altered, or any combinations of these factors.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment antigen-binding fragment” or “Fab fragment” as used herein refers to a fragment of an antibody that binds to antigens and that contains one antigen-binding site, one complete light chain, and part of one heavy chain. Fab is a monovalent fragment consisting of the VL, VH, CL and CH1 domains. Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

“F(ab′)₂ fragment” as used herein refers to antibodies generated by pepsin digestion of whole IgG antibodies to remove most of the Fc region while leaving intact some of the hinge region. F(ab′)₂ fragments have two antigen-binding F(ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoids unspecific binding to Fc receptor on live cells or to Protein A/G. F(ab′)₂ fragments can both bind and precipitate antigens.

“Favorable” as used herein means in certain aspects that, when the level(s) of one or more biomarkers measured or determined according to the methods described herein is compared to one or more reference levels, the measured level is less than or lower than the reference level(s). However, in other aspects, depending on the biomarker(s) being measured or determined, the term “favorable” may mean that the level(s) of the one or more biomarkers measured and/or determined according to the methods described herein is higher than or greater than the one or more reference levels. Whether a “favorable” level is higher or lower compared to the reference level depends on whether there is a rise or fall of the biomarker in the context of head injury. Generally, a favorable level of a biomarker is one that suggests an absence of head injury, improvement in a subject's health with respect to a head injury, or that a subject is likely to benefit from treatment (or continued treatment) for head injury. Methods for determining if the level(s) of one or more biomarkers measured and/or determined according to the methods described herein are favorable when compared to the reference level are well known in the art. For instance, one measure is clinical improvement of the subject which can be determined by any number of a variety of parameters (e.g., patient reports, etc.).

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpe.cam.ac.uk/) or in the international ImMunoGeneTics® (IMGT®) information system (hypertext transfer protocol://imgt.cines.fr/texts/IMGTrepertoire/LocusGenes/).

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

“GFAP” is used herein to describe glial fibrillary acidic protein. GFAP is a protein that is encoded by the GFAP gene in humans, and which can be produced (e.g., by recombinant means, in other species).

“Glasgow Coma Scale” or “GCS” as used herein refers to a 12 point scale for estimating and categorizing the outcomes of brain injury on the basis of overall social capability or dependence on others. The test measures the motor response, verbal response and eye opening response with these values: I. Motor Response (6—Obeys commands fully; 5—Localizes to noxious stimuli; 4—Withdraws from noxious stimuli; 3—Abnormal flexion, e.g., decorticate posturing; 2—Extensor response, e.g., decerebrate posturing; and 1—No response); II. Verbal Response (5—Alert and Oriented; 4—Confused, yet coherent, speech; 3—Inappropriate words and jumbled phrases consisting of words; 2—Incomprehensible sounds; and 1—No sounds); and III. Eye Opening (4—Spontaneous eye opening; 3—Eyes open to speech; 2—Eyes open to pain; and 1—No eye opening). The final score is determined by adding the values of I+II+III. The final score can be categorized into four possible levels for survival, with a lower number indicating a more severe injury and a poorer prognosis: Mild (13-15); Moderate Disability (9-12) (Loss of consciousness greater than 30 minutes; Physical or cognitive impairments which may or may resolve: and Benefit from Rehabilitation); Severe Disability (3-8) (Coma: unconscious state. No meaningful response, no voluntary activities); and Vegetative State (Less Than 3) (Sleep wake cycles; Arousal, but no interaction with environment; No localized response to pain). Moderate brain injury is defined as a brain injury resulting in a loss of consciousness from 20 minutes to 6 hours and a Glasgow Coma Scale of 9 to 12. Severe brain injury is defined as a brain injury resulting in a loss of consciousness of greater than 6 hours and a Glasgow Coma Scale of 3 to 8.

“Glasgow Outcome Scale” as used herein refers to a global scale for functional outcome that rates patient status into one of five categories: Dead, Vegetative State, Severe Disability, Moderate Disability or Good Recovery.

“Extended Glasgow Outcome Scale” or “GOSE” as used interchangeably herein provides more detailed categorization into eight categories by subdividing the categories of severe disability, moderate disability and good recovery into a lower and upper category as shown in Table 1.

TABLE 1

| | | | |
|---|---|---|---|
| 1 | Death | D | |
| 2 | Vegetative state | VX | |
| 3 | Lower severe disability | SD − | Condition of unawareness with only |
| 4 | Upper severe disability | SD + | reflex responses but with periods of |
| | | | spontaneous eye opening |
| 5 | Lower moderate | MD − | Patient who is dependent for daily |
| | disability | | support for mental or physical disability, |
| 6 | Upper moderate | MD + | usually a combination of both. If the |
| | disability | | patient can be left alone for more than |
| | | | 8 hours at home it is upper level of SD, |
| | | | if not then it is low level of SD. |
| 7 | Lower good recovery | GR − | Patients have some disability such as |
| 8 | Upper good recovery | GR + | aphasia, hemiparesis or epilepsy and/or |
| | | | deficits of memory or personality but are |
| | | | able to look after themselves. They are |
| | | | independent at home but dependent |
| | | | outside. If they are able to return to |
| | | | work even with special arrangement it is |
| | | | upper level of MD, if not then it is low |
| | | | level of MD. |

“Humanized antibody” is used herein to describe an antibody that comprises heavy and light chain variable region sequences from a non-human species (e.g., a mouse) but in which at least a portion of the VH and/or VL sequence has been altered to be more “human-like,” e.g., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (e.g., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

“Imaging procedure” as used herein refers to a medical test that allows the inside of a body to be seen in order to diagnose, treat, and monitor health conditions. An imaging procedure can be a non-invasive procedure that allows diagnosis of diseases and injuries without being intrusive. Examples of imaging procedures include MRI, CT scan, X-rays, positron emission tomography (PET) scan, single photon emission computed tomography (SPECT), and diffusion tensor imaging (DTI) scan.

“Injury to the head” or “head injury” as used interchangeably herein, refers to any trauma to the scalp, skull, or brain. Such injuries may include only a minor bump on the skull or may be a serious brain injury. Such injuries include primary injuries to the brain and/or secondary injuries to the brain. Primary brain injuries occur during the initial insult and result from displacement of the physical structures of the brain. More specifically, a primary brain injury is the physical damage to parenchyma (tissue, vessels) that occurs during the traumatic event, resulting in shearing and compression of the surrounding brain tissue. Secondary brain injuries occur subsequent to the primary injury and may involve an array of cellular processes. More specifically, a secondary brain injury refers to the changes that evolve over a period of time (from hours to days) after the primary brain injury. It includes an entire cascade of cellular, chemical, tissue, or blood vessel changes in the brain that contribute to further destruction of brain tissue.

An injury to the head can be either closed or open (penetrating). A closed head injury refers to a trauma to the scalp, skull or brain where there is no penetration of the skull by a striking object. An open head injury refers a trauma to the scalp, skull or brain where there is penetration of the skull by a striking object. An injury to the head may be caused by physical shaking of a person, by blunt impact by an external mechanical or other force that results in a closed or open head trauma (e.g., vehicle accident such as with an automobile, plane, train, etc.; blow to the head such as with a baseball bat, or from a firearm), a cerebral vascular accident (e.g., stroke), one or more falls (e.g., as in sports or other activities), explosions or blasts (collectively, “blast injuries”) and by other types of blunt force trauma. Alternatively, an injury to the head may be caused by the ingestion and/or exposure to a chemical, toxin or a combination of a chemical and toxin. Examples of such chemicals and/or toxins include fires, molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. Alternatively, an injury to the head may be caused as a result of a subject suffering from an autoimmune disease, a metabolic disorder, a brain tumor, one or more viruses, meningitis, hydrocephalus, hypoxia or any combinations thereof. In some cases, it is not possible to be certain whether any such event or injury has occurred or taken place. For example, there may be no history on a patient or subject, the subject may be unable to speak, the subject may be aware of what events they were exposed to, etc. Such circumstances are described herein as the subject “may have sustained an injury to the head.” In certain embodiments herein, the closed head injury does not include and specifically excludes a cerebral vascular accident, such as stroke.

“Intracranial lesion” as used herein refers to an area of injury within the brain. An intracranial lesion can be an abnormality seen on a imaging procedure or brain-imaging test, such as MRI or CT scan. On CT or MRI scans, brain lesions can appear as dark or light spots that do not look like normal brain tissue.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111In, 125I, 131I, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

In one aspect, the acridinium compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, e.g., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Linking sequences can be used for many purposes, including in recombinant Fabs. Exemplary linking sequences include, but are not limited to: (i) Histidine (His) tags, such as a 6×His tag, which has an amino acid sequence of HHHHHH (SEQ ID NO: 10), are useful as linking sequences to facilitate the isolation and purification of polypeptides and antibodies of interest; (ii) Enterokinase cleavage sites, like His tags, are used in the isolation and purification of proteins and antibodies of interest. Often, enterokinase cleavage sites are used together with His tags in the isolation and purification of proteins and antibodies of interest. Various enterokinase cleavage sites are known in the art. Examples of enterokinase cleavage sites include, but are not limited to, the amino acid sequence of DDDDK (SEQ ID NO: 11) and derivatives thereof (e.g., ADDDDK (SEQ ID NO: 12), etc.); (iii) Miscellaneous sequences can be used to link or connect the light and/or heavy chain variable regions of single chain variable region fragments. Examples of other linking sequences can be found in Bird et al., Science 242: 423-426 (1988); Huston et al., PNAS USA 85: 5879-5883 (1988); and McCafferty et al., Nature 348: 552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

“Monoclonal antibody” as used herein refers to an antibody obtained from a population of substantially homogeneous antibodies, e.g., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Multivalent binding protein” is used herein to refer to a binding protein comprising two or more antigen binding sites (also referred to herein as “antigen binding domains”). A multivalent binding protein is preferably engineered to have three or more antigen binding sites, and is generally not a naturally occurring antibody. The term “multispecific binding protein” refers to a binding protein that can bind two or more related or unrelated targets, including a binding protein capable of binding two or more different epitopes of the same target molecule.

“Negative predictive value” or “NPV” as used interchangeably herein refers to the probability that a subject has a negative outcome given that they have a negative test result.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). An “absolute amount” as used herein refers to the absolute value of a change or difference between at least two assay results taken or sampled at different time points and, which similar to a reference level, has been linked or is associated herein with various clinical parameters (e.g., presence of disease, stage of disease, severity of disease, progression, non-progression, or improvement of disease, etc.). “Absolute value” as used herein refers to the magnitude of a real number (such as, for example, the difference between two compared levels (such as levels taken at a first time point and levels taken at a second time point)) without regard to its sign, e.g., regardless of whether it is positive or negative.

This disclosure provides exemplary reference levels and absolute amounts (e.g., calculated by comparing reference levels at different time points). However, it is well-known that reference levels and absolute amounts may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels and absolute amounts for those other immunoassays based on the description provided by this disclosure. Whereas the precise value of the reference level and absolute amount may vary between assays, the findings as described herein should be generally applicable and capable of being extrapolated to other assays.

“Point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long term care and/or hospice facility). Examples of point-of-care devices include those produced by Abbott Laboratories (Abbott Park, Ill.) (e.g., i-STAT and i-STAT Alinity, Universal Biosensors (Rowville, Australia) (see US 2006/0134713), Axis-Shield PoC AS (Oslo, Norway) and Clinical Lab Products (Los Angeles, USA).

“Positive predictive value” or “PPV” as used interchangeably herein refers to the probability that a subject has a positive outcome given that they have a positive test result.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low”, “medium”, or “high” levels), can be used. Multiple calibrators (e.g., more than one calibrator or a varying amount of calibrator(s)) can be used in conjunction to comprise a “sensitivity panel.”

A “receiver operating characteristic” curve or “ROC” curve refers to a graphical plot that illustrates the performance of a binary classifier system as its discrimination threshold is varied. For example, an ROC curve can be a plot of the true positive rate against the false positive rate for the different possible cutoff points of a diagnostic test. It is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate. The ROC curve demonstrates the tradeoff between sensitivity and specificity (any increase in sensitivity will be accompanied by a decrease in specificity); the closer the curve follows the left-hand border and then the top border of the ROC space, the more accurate the test; the closer the curve comes to the 45-degree diagonal of the ROC space, the less accurate the test; the slope of the tangent line at a cutoff point gives the likelihood ratio (LR) for that value of the test; and the area under the curve is a measure of test accuracy.

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described in (i) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, e.g., the bifunctional antibodies have a dual specificity.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “test sample,” “specimen,” “sample from a subject,” and “patient sample” as used herein may be used interchangeable and may be a sample of blood such as whole blood, tissue, urine, serum, plasma, amniotic fluid, cerebrospinal fluid, placental cells or tissue, endothelial cells, leukocytes, or monocytes. In some embodiments, the sample is a whole blood sample. In some embodiments, the sample is a serum sample. In yet other embodiments, the sample is a plasma sample. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

A variety of cell types, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, red blood cells, platelets, interstitial fluid, cerebral spinal fluid, etc. Cell types and tissues may also include lymph fluid, cerebrospinal fluid, a fluid collected by A tissue or cell type may be provided by removing a sample of cells from a human and a non-human animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

“Sensitivity” of an assay as used herein refers to the proportion of subjects for whom the outcome is positive that are correctly identified as positive (e.g., correctly identifying those subjects with a disease or medical condition for which they are being tested). For example, this might include correctly identifying subjects as having a TBI from those who do not have a TBI, correctly identifying subjects having a moderate, severe, or moderate to severe TBI from those having a mild TBI, correctly identifying subjects as having a mild TBI from those having a moderate, severe, or moderate to severe TBI, correctly identifying subjects as having a moderate, severe, or moderate to severe TBI from those having no TBI or correctly identifying subjects as having a mild TBI from those having no TBI, correctly identifying subjects as likely to benefit from conventional MRI procedure or an advanced MRI procedure from those who are not likely to benefit from conventional MRI procedure or advanced MRI procedure, etc.).

“Specificity” of an assay as used herein refers to the proportion of subjects for whom the outcome is negative that are correctly identified as negative (e.g., correctly identifying those subjects who do not have a disease or medical condition for which they are being tested). For example, this might include correctly identifying subjects having an TBI from those who do not have a TBI, correctly identifying subjects not having a moderate, severe, or moderate to severe TBI from those having a mild TBI, correctly identifying subjects as not having a mild TBI from those having a moderate, severe, or moderate to severe TBI or correctly identifying subjects as not having any TBI, or correctly identifying subjects as having a mild TBI from those having no TBI, etc.).

“Series of calibrating compositions” refers to a plurality of compositions comprising a known concentration of a biomarker (such as, for example, UCH-L1, GFAP, NSE, APOA1, S100β, etc.), wherein each of the compositions differs from the other compositions in the series by the concentration of the biomarker (such as for example, UCH-L1, GFAP, SNE, APOA1, S100β, etc.).

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistical significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. In some embodiments, the subject is a human. The subject or patient may be undergoing other forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Traumatic Brain Injury” or “TBI” as used interchangeably herein refers to a complex injury with a broad spectrum of symptoms and disabilities. TBI is most often an acute event similar to other injuries. TBI can be classified as “mild,” “moderate,” or “severe.” The causes of TBI are diverse and include, for example, physical shaking by a person, a car accident, injuries from firearms, cerebral vascular accidents (e.g., strokes), falls, explosions or blasts and other types of blunt force trauma. Other causes of TBI include the ingestion and/or exposure to one or more chemicals or toxins (such as fires, molds, asbestos, pesticides and insecticides, organic solvents, paints, glues, gases (such as carbon monoxide, hydrogen sulfide, and cyanide), organic metals (such as methyl mercury, tetraethyl lead and organic tin), one or more drugs of abuse or combinations thereof). Alternatively, TBI can occur in subjects suffering from an autoimmune disease, a metabolic disorder, a brain tumor, hypoxia, one or more viruses, meningitis, hydrocephalus or combinations thereof. Young adults and the elderly are the age groups at highest risk for TBI. In certain embodiments herein, traumatic brain injury or TBI does not include and specifically excludes cerebral vascular accidents such as strokes.

“Mild TBI” as used herein refers to a brain injury where loss of consciousness is brief and usually a few seconds or minutes and/or confusion and disorientation is shorter than 1 hour. Mild TBI is also referred to as a concussion, minor head trauma, minor TBI, minor brain injury, and minor head injury. While MRI scans are often normal, the individual with mild TBI may have cognitive problems such as headache, difficulty thinking, memory problems, attention deficits, mood swings and frustration.

Mild TBI is the most prevalent TBI and is often missed at time of initial injury. Typically, a subject has a Glasgow Coma scale number of between 13-15 (such as 13-15 or 14-15). Fifteen percent (15%) of people with mild TBI have symptoms that last 3 months or more. Mild TBI is defined as the result of the forceful motion of the head or impact causing a brief change in mental status (confusion, disorientation or loss of memory) or loss of consciousness for less than 30 minutes. Common symptoms of mild TBI include fatigue, headaches, visual disturbances, memory loss, poor attention/concentration, sleep disturbances, dizziness/loss of balance, irritability-emotional disturbances, feelings of depression, and seizures. Other symptoms associated with mild TBI include nausea, loss of smell, sensitivity to light and sounds, mood changes, getting lost or confused, and/or slowness in thinking.

“Moderate TBI” as used herein refers to a brain injury where loss of consciousness and/or confusion and disorientation is between 1 and 24 hours and the subject has a Glasgow Coma scale number of between 9-13 (such as 9-12 or 9-13). The individual with moderate TBI have abnormal brain imaging results. “Severe TBI” as used herein refers to a brain injury where loss of consciousness is more than 24 hours and memory loss after the injury or penetrating skull injury longer than 24 hours and the subject has a Glasgow Coma scale number between 3-8. The deficits range from impairment of higher level cognitive functions to comatose states. Survivors may have limited function of arms or legs, abnormal speech or language, loss of thinking ability or emotional problems. Individuals with severe injuries can be left in long-term unresponsive states. For many people with severe TBI, long-term rehabilitation is often necessary to maximize function and independence.

“Moderate to severe” TBI as used herein refers to a spectrum of brain injury that includes moderate to severe and thus encompasses moderate TBI alone, severe TBI alone and moderate to severe TBI combined. Subjects suffering from a moderate to severe TBI can have a Glasgow Coma scale number of between 3-13 (such as 3-12 or 3-13). For example, in some clinical situations, a subject may initially be diagnosed as having a moderate TBI but who, over the course of time (minutes, hours or days), progress to having a severe TBI (such, as for example, in situations when there is a brain bleed). Such subjects would be examples of patients that could be classified as “moderate to severe”. Common symptoms of moderate to severe TBI include cognitive deficits including difficulties with attention, concentration, distractibility, memory, speed of processing, confusion, perseveration, impulsiveness, language processing, and/or “executive functions”, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine discrimination, the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), hearing, such as decrease or loss of hearing, ringing in the ears (tinnitus), increased sensitivity to sounds, loss or diminished sense of smell (anosmia), loss or diminished sense of taste, the convulsions associated with epilepsy that can be several types and can involve disruption in consciousness, sensory perception, or motor movements, control of bowel and bladder, sleep disorders, loss of stamina, appetite changes, regulation of body temperature, menstrual difficulties, dependent behaviors, emotional ability, lack of motivation, irritability, aggression, depression, disinhibition, or denial/lack of awareness.

“Ubiquitin carboxy-terminal hydrolase L1” or “UCH-L1” as used interchangeably herein refers to a deubiquitinating enzyme encoded by the UCH-L1 gene in humans. UCH-L1, also known as ubiquitin carboxyl-terminal esterase L1 and ubiquitin thiolesterase, is a member of a gene family whose products hydrolyze small C-terminal adducts of ubiquitin to generate the ubiquitin monomer.

“Unfavorable” as used herein means in certain aspects that when the level(s) of one or more biomarkers measured or determined according to the methods described herein is compared to one or more reference levels, the measured level is higher or greater than the reference level(s). However, in other aspects, depending on the biomarker(s) being measured or determined, the term “unfavorable” may mean that the level(s) of the one or more biomarkers measured or determined according to the methods described herein is less or lower than the one or more reference levels. As described above, whether an “unfavorable” level is higher or lower compared to the reference level depends on whether there is a rise or fall of the biomarker in the context of head injury. Generally, an unfavorable level of a biomarker is one that suggests a presence of head injury, worsening of a subject's health with respect to a head injury, or that a subject is not likely to benefit from treatment (or continued treatment) with therapy or treatment for head injury. Methods for determining if the level(s) of one or more biomarkers measured or determined according to the methods described herein are unfavorable when compared to the reference level are well known in the art. For instance, one measure is a worsening of the clinical condition of the subject which can be determined by any number of a variety of parameters (e.g., patient reports, etc.).

“Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, e.g., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-biomarker (e.g., NSE, GFAP, UCH-L1, S100β, etc.) antibody that differs from the corresponding fragment of anti-biomarker (e.g., NSE, GFAP, UCH-L1, S100β, etc.) antibody in amino acid sequence but is still antigenically reactive and can compete with the corresponding fragment of anti-biomarker (e.g., NSE, GFAP, UCH-L1, S100β, etc.) antibody for binding with UCH-L1. “Variant” also can be used to describe a polypeptide or a fragment thereof that has been differentially processed, such as by proteolysis, phosphorylation, or other post-translational modification, yet retains its antigen reactivity.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Methods of Aiding in the Determination of Whether to Perform an Advanced Magnetic Resonance Imaging (MRI) Procedure on a Human Subject Who has Sustained or May Have Sustained an Injury to the Head Using One Sample from the Subject

The present disclosure relates, among other methods, to a method of aiding in determining whether to perform an advanced magnetic resonance (MRI) imaging procedure on a human subject who has sustained or may have sustained an injury to the head. As used herein, “determination of whether to perform an advanced MRI procedure on a human subject” refers to the fact that the aforementioned method can be used, e.g., with other information (e.g., clinical assessment data), to determine that the subject is more likely than not to have a positive advanced MRI scan, i.e., the presence of an intracranial lesion. Specifically, such a method can comprise the steps of: (a) performing an assay a sample obtained from the subject after an injury or suspected injury to the head subject (such as within about 24 hours, within about 48 hours, or within 72 hours) to measure or detect a level of one or more biomarkers (for example, one biomarker, two biomarkers, three biomarkers, four biomarkers, five biomarkers, six biomarkers, seven biomarkers, eight biomarkers, nine biomarkers, ten biomarkers, eleven biomarkers, twelve biomarkers, thirteen biomarkers, fourteen biomarkers, fifteen biomarkers, sixteen biomarkers, seventeen biomarkers, eighteen biomarkers, nineteen biomarkers, twenty biomarkers, etc.) that provide a measure or indication of TBI; and (b) performing an advanced MRI procedure (scan) on the subject when the level of the one or more biomarkers in the sample is unfavorable (e.g, has increased or decreased; is higher or lower) when compared to a reference level of the early biomarker (“rule in” performing an imaging procedure) and not performing an advanced MRI procedure on the subject when the level of the one or more biomarkers in the sample is favorable (e.g., has decreased or increased; is lower or higher) a reference level of the early biomarker (“rule out” performing an imaging procedure). The sample can be a biological sample.

Any biomarkers which can provide a measure or indication of TBI can be used in the methods described herein. Examples of biomarkers that provide a measure or indication of TBI can be: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In another aspect, one or more biomarkers that can be used in the methods described herein include: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

When the amount of one or more biomarkers is measured, this amount can be compared to one or more reference levels (e.g., if one biomarker is measured (e.g., NF-L)), then one reference level will be needed (e.g., NF-L), if two biomarkers are measured (e.g., APOA1 and MBP), then two reference levels will be needed (e.g., one for APOA1 and one for MBP), if three biomarkers are measured (e.g., NSE, tau, hsCRP, then three reference levels will be needed (one for NSE, one for tau and one for hsCRP), etc.

Generally, a reference level of one or more biomarkers can be employed as a benchmark against which to assess results obtained upon assaying a test sample for the one or more biomarkers. Generally, in making such a comparison, the reference level of the one or more biomarkers is obtained by running a particular assay a sufficient number of times and under appropriate conditions such that a linkage or association of analyte presence, amount or concentration with a particular stage or endpoint of TBI or with particular indicia can be made. Typically, the reference level of the one or more biomarkers is obtained with assays of reference subjects (or populations of subjects). The one or more biomarkers measured can include fragments thereof, degradation products thereof, and/or enzymatic cleavage products thereof.

Any advanced MRI procedure can be used in the methods described herein. Examples of at least one advanced MRI procedure that can be performed on the subject include gradient echo magnetic resonance imaging, echo planar magnetic resonance imaging, spin echo magnetization resonance imaging, diffusion MRI (e.g, multi-shell MRI), resting-state functional MRI (fMRI), magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

In some embodiments, the sample may be obtained or taken from the subject within about 0 minutes, within about 1 minute, within about 2 minutes, within about 3 minutes, within about 4 minutes, within about 5 minutes, within about 6 minutes, within about 7 minutes, within about 8 minutes, within about 9 minutes, within about 10 minutes, within about 11 minutes, within about 12 minutes, within about 13 minutes, within about 14 minutes, within about 15 minutes, within about 20 minutes, within about 30 minutes, within about 1 hour, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, within about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of a head injury or suspect injury to the head.

If more than one sample is obtained from the subject, the sample can be obtained at various time points after the first sample was obtained (e.g., such as about 1 minute after the first or subsequent sample was taken, about 2 minutes after the first or subsequent sample was taken, about 3 minutes after the first or subsequent sample was taken, about 4 minutes after the first or subsequent sample was taken, about 5 minutes after the first or subsequent sample was taken, about 10 minutes after the first or subsequent sample was taken, about 30 minutes after the first or subsequent sample was taken, about 45 minutes after the first or subsequent sample was taken, about 1 hour after the first or subsequent sample was taken, about 2 hours after the first or subsequent sample was taken, about 3 hours after the first or subsequent sample was taken, about 4 hours after the first or subsequent sample was taken, about 5 hours after the first or subsequent sample was taken, about 6 hours after the first or subsequent sample was taken, about 7 hours after the first or subsequent sample was taken, about 8 hours after the first or subsequent sample was taken, about 9 hours after the first or subsequent sample was taken, about 10 hours after the first or subsequent sample was taken, about 11 hours after the first or subsequent sample was taken, about 12 hours after the first or subsequent sample was taken, etc.

In some embodiments, the subject has received an imaging procedure, such as a conventional MRI procedure or advanced MRI procedure, before or after the assay is performed. In some embodiments, the subject is suspected as having a traumatic brain injury based on the imaging procedure. In some embodiments, the reference level of the one or more biomarkers is correlated with a positive MRI scan i.e., the presence of an intracranial lesion.

In some embodiments, the subject has received a Glasgow Coma Scale score before or after the at least one assay, at least one advanced MRI procedure, or the at least one assay and at least one advanced MRI procedure is performed.

In some embodiments, the method further includes using the data obtained from the one or more biomarkers and/or the advanced MRI procedure performed on a subject along with other clinical information obtained from the subject (e.g., such as, for example, a Glasgow Coma Scale Score, a determination of the pathophysiology of the head injury (e.g., whether the subject is suffering from one or more of cerebral hypofusion or hyperfusion, cerebral ischaemia, hyperaemia, impaired cerebrovascular CO₂-reactivity (such as, for example, cerebrovascular constriction or dilation in response to hypo- or hypercapnia), a cerebral vasospasm, brain tissue hypoxia, oedema (such as vasogenic brain or cytotoxic brain oedema), oxidative stress, cell death (such as, for example, necrosis or apoptosis) or any combinations thereof) the endotype of the head injury (e.g., whether the injury is axonal, neuronal or astroglial, whether there is microvascular injury, whether there is proteinopathy, is there inflammation or any combinations thereof of the injury), the type of TBI (e.g., mild, moderate, severe, or moderate to severe TBI) and/or any combinations thereof) to determine the degree of impairment in one or more systems in the brain. Determination of the degree of impairment will guide the physician in making treatment decisions for the subject as will be discussed in further detail herein.

In some embodiments, the method further includes treating the human subject with a traumatic brain injury treatment and/or monitoring the human subject, as described below.

The nature of the assay employed in the methods described herein is not critical and the test can be any assay known in the art such as, for example, immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in a clinical chemistry format such as would be known by one of ordinary skill in the art. Such assays are described in further detail herein in Sections 8-11. It is known in the art that the values (e.g., reference levels, cutoffs, thresholds, specificities, sensitivities, concentrations of calibrators and/or controls etc.) used in an assay that employs specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4^{th} edition, chapter 3.5, pages 315-322, the contents of which are herein incorporated by reference).

### 3. Methods of Evaluating or Aiding in the Diagnosis of a Human Subject for a Head Injury

The present disclosure relates, among other methods, to a method of evaluating or aiding in the diagnosis and evaluation of whether a human subject has sustained or may have sustained an injury to the head and, if so, whether the subject has sustained a traumatic brain injury (TBI). For example, the method described herein can aid in determining that a subject suspected of a head injury (e.g., injury to the head) has in fact, not suffered a head injury. Alternatively, the method can aid in determining that a subject with a suspected head injury has in fact suffered a head injury, but not a TBI. Still further, the method can aid in determining that a subject with a suspected head injury has in fact suffered head injury, specifically, a TBI. Additionally, for subjects determined to have suffered a head injury, the method can be used to assess (i) the pathophysiology of the head injury (e.g., whether the subject has one or more of a cerebral hypofusion or hyperfusion, cerebral ischaemia, hyperaemia, impaired cerebrovascular CO₂-reactivity (such as, for example, cerebrovascular constriction or dilation in response to hypo- or hypercapnia), a cerebral vasospasm, brain tissue hypoxia, oedema (such as vasogenic brain or cytotoxic brain oedema), oxidative stress, cell death (such as, for example, necrosis or apoptosis) or any combinations thereof, the final determination can identify or characterize the subject as having or suffering or at risk of suffering from one or more of cerebral hypofusion or hyperfusion, cerebral ischaemia, hyperaemia, impaired cerebrovascular CO₂-reactivity (such as, for example, cerebrovascular constriction or dilation in response to hypo- or hypercapnia), a cerebral vasospasm, brain tissue hypoxia, oedera (such as vasogenic brain or cytotoxic brain oedema), oxidative stress, cell death (such as, for example, necrosis or apoptosis) or any combinations thereof); (ii) the endotype of the head injury (e.g., identifying that the injury is axonal, neuronal or astroglial, whether there is microvascular injury, whether there is proteinopathy, is there inflammation or any combinations thereof); (iii) whether the subject has, or more likely than not has, sustained a mild, moderate, severe, or moderate to severe TBI; or (iv) any combinations of (i)-(iii). Information on the pathophysiology of the head injury, the endotype of the head injury and/or the type of TBI (mild, moderate, severe, or moderate to severe TBI) assessed using the methods described herein can be used to determine the degree of impairment in one or more systems of the brain and guide and manage treatment decisions and options for the subject, monitoring of the subject and/or predicting the outcome of the subject as discussed in further detail herein.

The method described herein involves: (1) detecting or determining the presence or amount of one or more biomarkers in one or more samples obtained from the subject (namely, one who has or is suspected of suffering an injury to the head); (2) obtaining data from the subject using at least one advanced magnetic resonance imaging procedure; and (3) processing all or part of the information and data obtained from the biomarkers and/or at least one advanced MRI procedure using a computer-implemented method.

The method involves performing at least one assay on one or more samples (e.g., such as a first sample, a second sample, etc.) obtained from the human subject (such as within about 24 hours, within about 48 hours, or within 72 hours) after a suspected injury to the head to determine the presence of (e.g., detect) or measure a level of one or more biomarkers (for example, one biomarker, two biomarkers, three biomarkers, four biomarkers, five biomarkers, six biomarkers, seven biomarkers, eight biomarkers, nine biomarkers, ten biomarkers, eleven biomarkers, twelve biomarkers, thirteen biomarkers, fourteen biomarkers, fifteen biomarkers, sixteen biomarkers, seventeen biomarkers, eighteen biomarkers, nineteen biomarkers, twenty biomarkers, etc.) in the sample using routine techniques known in the art. If more than one sample is obtained from the subject, the sample can be obtained at various time points after the first sample was obtained (e.g., such as about 1 minute after the first or subsequent sample was taken, about 2 minutes after the first or subsequent sample was taken, about 3 minutes after the first or subsequent sample was taken, about 4 minutes after the first or subsequent sample was taken, about 5 minutes after the first or subsequent sample was taken, about 10 minutes after the first or subsequent sample was taken, about 30 minutes after the first or subsequent sample was taken, about 45 minutes after the first or subsequent sample was taken, about 1 hour after the first or subsequent sample was taken, about 2 hours after the first or subsequent sample was taken, about 3 hours after the first or subsequent sample was taken, about 4 hours after the first or subsequent sample was taken, about 5 hours after the first or subsequent sample was taken, about 6 hours after the first or subsequent sample was taken, about 7 hours after the first or subsequent sample was taken, about 8 hours after the first or subsequent sample was taken, about 9 hours after the first or subsequent sample was taken, about 10 hours after the first or subsequent sample was taken, about 11 hours after the first or subsequent sample was taken, about 12 hours after the first or subsequent sample was taken, etc.

Any biomarkers which can provide a measure or indication of TBI can be used in the methods described herein. Examples of biomarkers that provide a measure or indication of TBI can be: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In another aspect, one or more biomarkers that can be used in the methods described herein include: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

When the amount of one or more biomarkers is measured, this amount can be compared to one or more reference levels (e.g., if one biomarker is measured (e.g., NF-L)), then one reference level will be needed (e.g., NF-L), if two biomarkers are measured (e.g., APOA1 and MBP), then two reference levels will be needed (e.g., one for APOA1 and one for MBP), if three biomarkers are measured (e.g., NSE, tau, hsCRP then three reference levels will be needed (one for NSE, one for tau and one for hsCRP), etc. Generally, a reference level of one or more biomarkers can be employed as a benchmark against which to assess results obtained upon assaying a test sample for the one or more biomarkers. Generally, in making such a comparison, the reference level of the one or more biomarkers is obtained by running a particular assay a sufficient number of times and under appropriate conditions such that a linkage or association of analyte presence, amount or concentration with a particular stage or endpoint of TBI or with particular indicia can be made. Typically, the reference level of the one or more biomarkers is obtained with assays of reference subjects (or populations of subjects). The one or more biomarkers measured can include fragments thereof, degradation products thereof, and/or enzymatic cleavage products thereof.

In certain embodiments, the reference level may be correlated with control subjects that have not sustained a head injury.

In one aspect, a (preliminary) first TBI diagnosis is made based on the amount of the one or more biomarkers determined (e.g., measured) by the assay and optionally, whether the amount of the one or more biomarkers have increased or decreased when compared to the one or more reference levels. For example, the (preliminary) first TBI diagnosis may be based on the amount of the one or more biomarkers determined or measured in the one or more samples obtained from the subject (and optionally, whether they have increased or decreased when compared to one or more reference levels) to determine that the subject more likely than not: (1) does not have a head injury; (2) has a head injury but does not have a TBI; (3) has a head injury and has a TBI; (4) has a head injury and has a mild TBI; or (5) has a head injury and has a moderate, severe, or moderate to severe TBI.

In another aspect, the amount (data) of the one or more biomarkers determined (e.g., measured) by the at least one assay can be used in a computer-implemented method to generate a single TBI diagnosis as discussed in more detail below.

In addition to performing the at least one assay on one or more samples taken from the subject, the method described herein further involves performing at least one advanced MRI procedure on the subject (such as within 24 hours, within 48 hours or within 72 hours). The at least one advanced MRI procedure performed on the subject can be gradient echo magnetic resonance imaging, echo planar magnetic resonance imaging, spin echo magnetization resonance imaging, diffusion MRI (e.g., multi-shell diffusion MRI), resting-state functional MRI, magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof. The advanced MRI procedure performed generates data on the brain of the subject, such as, for example, diffusion MRI data (e.g., multi-shell diffusion MRI), resting-state functional MRI data, etc.

In one aspect, the data obtained from the at least one advanced MRI procedure can be used in a computer-implemented method to generate a (preliminary) second TBI diagnosis for the subject. Exemplary computer-implemented methods that can be used to generate the (preliminary) second TBI diagnosis include those disclosed in U.S. Pat. No. 9,265,441, the contents of each of which are herein incorporated by reference. Alternatively, another example is the READY View image analysis software available from GE Healthcare (GE Medical Systems SCS) (Buc, France) that allows users to process dynamic or functional volumetric data and to generate maps that display changes in image intensity over time, echo time, b-value and frequency, can also be used. For example, the advanced MRI techniques can provide information on the structural and functional connectivity of the subject's brain. However, it should be knotted that any computer-implemented method that employs data obtained from at least one advanced MRI procedure can be used in the methods of the present invention.

Once the first and second TBI diagnoses are generated, these diagnoses are combined to generate a final TBI diagnosis (final determination). The final determination can be that the subject: (1) does not have a head injury; (2) has a head injury but does not have a TBI; (3) has a head injury and has a TBI. Additionally, for subjects identified as having a head injury and a TBI, the final determination also provides (i) the pathophysiology of the head injury; (ii) the endotype of the head injury; (iii) whether the subject has, or more likely than not has, sustained a mild, moderate, severe, or moderate to severe TBI; or (iv) any combinations of (i)-(iii). For example, with respect to the pathophysiology of the injury, the final determination can identify or characterize the subject as having or suffering or at risk of suffering from one or more of cerebral hypofusion or hyperfusion, cerebral ischaemia, hyperaemia, impaired cerebrovascular CO₂-reactivity (such as, for example, cerebrovascular constriction or dilation in response to hypo- or hypercapnia), a cerebral vasospasm, brain tissue hypoxia, oedema (such as vasogenic brain or cytotoxic brain oedema), oxidative stress, cell death (such as, for example, necrosis or apoptosis) or any combinations thereof. With respect to the endotype of the injury, the final determination identifies whether the injury is axonal, neuronal or astroglial, whether there is microvascular injury, whether there is proteinopathy, is there inflammation or any combinations thereof.

In another aspect, the amount of the one or more biomarkers determined by the assay and the data obtained from the at least one advanced MRI procedure can be used in a computer-implemented method to generate a TBI diagnosis for the subject. The TBI diagnosis can be that the subject more likely than not: (1) does not have a head injury; (2) has a head injury but does not have a TBI; or (3) has a head injury and has a TBI. Additionally, for subjects identified as having a head injury and a TBI, the final determination also provides (i) the pathophysiology of the head injury; (ii) the endotype of the head injury; (iii) whether the subject has, or more likely than not has, sustained a mild, moderate, severe, or moderate to severe TBI; or (iv) any combinations of (i)-(iii). For example, with respect to the pathophysiology of the injury, the final determination can identify or characterize the subject as having or suffering or at risk of suffering from one or more of cerebral hypofusion or hyperfusion, cerebral ischaemia, hyperaemia, impaired cerebrovascular CO₂-reactivity (such as, for example, cerebrovascular constriction or dilation in response to hypo- or hypercapnia), a cerebral vasospasm, brain tissue hypoxia, oedema (such as vasogenic brain or cytotoxic brain oedema), oxidative stress, cell death (such as, for example, necrosis or apoptosis) or any combinations thereof. With respect to determining the endotype of the injury, the TBI diagnosis identifies whether the injury is axonal, neuronal or astroglial, whether there is microvascular injury, whether there is proteinopathy, is there inflammation or any combinations thereof. Additionally, information on the pathophysiology of the head injury, the endotype of the head injury and/or the type of TBI (mild, moderate, severe, or moderate to severe) determined using the methods described herein can be used to determine the degree of impairment in one or more systems of the brain and guide and manage treatment decisions and options for the subject, monitoring of the subject and/or predicting the outcome of the subject.

The at least one advanced MRI procedure can be performed on the subject either prior to or after the at least one assay is performed. In fact, the order in which the at least one assay or advanced MRI procedure are performed is not critical.

In some embodiments, the sample may be obtained or taken from the subject within about 0 minutes, within about 1 minute, within about 2 minutes, within about 3 minutes, within about 4 minutes, within about 5 minutes, within about 6 minutes, within about 7 minutes, within about 8 minutes, within about 9 minutes, within about 10 minutes, within about 11 minutes, within about 12 minutes, within about 13 minutes, within about 14 minutes, within about 15 minutes, within about 20 minutes, within about 30 minutes, within about 1 hour, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, within about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of a suspect injury to the head.

In some embodiments, the subject has received a Glasgow Coma Scale score before or after the at least one assay, at least one advanced MRI procedure, or the at least one assay and at least one advanced MRI procedure is performed. In some embodiments, the subject is suspected as having a moderate, severe, or a moderate to severe traumatic brain injury based on the Glasgow Coma Scale score. In some embodiments, the reference level of at least one biomarker is correlated with subjects having moderate to severe traumatic brain injury. In some embodiments, the reference level of the at least one biomarker is correlated with a Glasgow Coma Scale score of 9-13 (a moderate TBI). In some embodiments, the reference level of the biomarker is correlated with a Glasgow Coma Scale score of 3-8 (a severe TBI). In some embodiments, the reference level of the biomarker is correlated with a Glasgow Coma Scale score of 3-12 (a moderate to severe TBI). In some embodiments, the subject is suspected as having mild traumatic brain injury based on the Glasgow Coma Scale score. In some embodiments, the reference level of the biomarker is correlated with subjects having mild traumatic brain injury. In some embodiments, the reference level of the biomarker is correlated with a Glasgow Coma Scale score of 13-15 (mild TBI).

In some embodiments, the method further includes treating a human subject assessed as having a traumatic brain injury with a traumatic brain injury treatment, as described below. In yet other embodiments, the method further includes monitoring a subject assessed with a traumatic brain injury with a traumatic brain injury treatment. In some embodiments, the method further includes monitoring a human subject assessed as having traumatic brain injury, as described below.

The nature of the assay employed in the methods described herein is not critical and the test can be any assay known in the art such as, for example, immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Nonetheless, tests or assays competent to perform the claimed methods will be employed, such as, for example, assays having various sensitivities and sensitivities as described herein. Moreover, the assays employed in the methods described herein can be employed in a clinical chemistry format such as would be known by one of ordinary skill in the art. Such assays are described in further detail herein in Section 8. It is known in the art that the values (e.g., reference levels, cutoffs, thresholds, specificities, sensitivities, concentrations of calibrators and/or controls etc.) used in an assay that employs specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4^{th} edition, chapter 3.5, pages 315-322, the contents of which are herein incorporated by reference).

### 4. Methods of Predicting or Aiding in Predicting of the Outcome of Human Subjects Who have a Traumatic Brain Injury

The present disclosure relates, among other methods, to a method for predicting or as an aid in predicting the outcome of human subjects that have suffered a traumatic brain injury, such as, for example, a mild, moderate, severe, or moderate to severe TBI. “Predicting the outcome of a human subject that has suffered a TBI” refers to determining whether the subject will have an unfavorable outcome or a favorable outcome. As used herein, “determining whether the subject has an unfavorable outcome or a favorable outcome” refers to the fact that the aforementioned method can be used, e.g., with other information (such as, but not limited to, clinical assessment data), to determine that the subject is more likely than not to have an negative outcome (e.g., such as the subject will make only a partial or marginal recovery (meaning that the subject may be partially or permanently disabled or impaired from the TBI and may need long term therapeutic intervention and/or care and/or the subject will experience a poor, reduced and/or diminished quality of life) or a positive outcome (e.g., such as the subject will make a complete recovery from a TBI and/or having a normal and/or full quality of life).

In one aspect, the method described herein involves: (1) performing at least one assay on a sample taken from the subject that has suffered (e.g., been diagnosed with) a TBI; (2) performing at least one advanced MRI procedure on the subject when the level of the one or more biomarkers in the sample is higher than a reference level of the one or more biomarkers; and (3) predicting an outcome in the human subject based on the results of the advanced MRI procedure. Specifically, in this method, the advanced MRI procedure identifies the location of the TBI in the brain of the subject. For example, the location of the TBI may provide insight and guidance as to the prognosis of the subject. For example, the location of the TBI may predispose the subject to depression, another location may predispose the subject to Alzheimer's disease, etc. Depending on the location of the TBI as provided by the advanced TBI imaging, it can be predicted that the subject may have a favorable outcome or an unfavorable outcome as a result of the TBI.

Alternatively, the method described herein can be used to predict the outcome in a subject suffering from stress, particularly post-traumatic stress syndrome (PTSD). For example, it is known in the art that stress, particularly, PTSD is associated with changes in the hippocampus, amygdala, and medial prefrontal cortex of the brain. Advanced MRI procedures can be used to identify such changes in the brain. Thus, in this aspect, the method would involve (1) performing at least one assay on a sample taken from the subject that has suffered (e.g., been diagnosed with) a stress (such as PTSD); (2) performing at least one advanced MRI procedure on the subject when the level of the one or more biomarkers in the sample is higher than a reference level of the one or more biomarkers; and (3) predicting an outcome in the human subject based on the results of the advanced MRI procedure.

In another aspect, the method described herein involves: (1) detecting or determining the presence or amount of one or more biomarkers in as sample obtained from the subject that has suffered (e.g., been diagnosed with) a TBI; (2) obtaining data from the subject using at least one advanced magnetic resonance imaging procedure; and (3) processing all or part of the information and data obtained from the biomarkers and/or at least one advanced MRI procedure using a computer-implemented method.

The method involves performing at least one assay on one or more samples (e.g., such as a first sample, a second sample, etc.) obtained from the human subject (such as within about 24 hours, within about 48 hours or within 72 hours) after diagnosis of a TBI to determine the presence of (e.g., detect) or measure a level of one or more biomarkers (for example, one biomarker, two biomarkers, three biomarkers, four biomarkers, five biomarkers, six biomarkers, seven biomarkers, eight biomarkers, nine biomarkers, ten biomarkers, eleven biomarkers, twelve biomarkers, thirteen biomarkers, fourteen biomarkers, fifteen biomarkers, sixteen biomarkers, seventeen biomarkers, eighteen biomarkers, nineteen biomarkers, twenty biomarkers, etc.) in the sample using routine techniques known in the art. If more than one sample is obtained from the subject, the sample can be obtained at various time points after the first sample was obtained (e.g., such as about 1 minute after the first or subsequent sample was taken, about 2 minutes after the first or subsequent sample was taken, about 3 minutes after the first or subsequent sample was taken, about 4 minutes after the first or subsequent sample was taken, about 5 minutes after the first or subsequent sample was taken, about 10 minutes after the first or subsequent sample was taken, about 30 minutes after the first or subsequent sample was taken, about 45 minutes after the first or subsequent sample was taken, about 1 hour after the first or subsequent sample was taken, about 2 hours after the first or subsequent sample was taken, about 3 hours after the first or subsequent sample was taken, about 4 hours after the first or subsequent sample was taken, about 5 hours after the first or subsequent sample was taken, about 6 hours after the first or subsequent sample was taken, about 7 hours after the first or subsequent sample was taken, about 8 hours after the first or subsequent sample was taken, about 9 hours after the first or subsequent sample was taken, about 10 hours after the first or subsequent sample was taken, about 11 hours after the first or subsequent sample was taken, about 12 hours after the first or subsequent sample was taken, etc.

Any biomarkers which can provide a measure or indication of TBI can be used in the methods described herein. Examples of biomarkers of TBI can be: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In another aspect, one or more biomarkers that can be used in the methods described herein include: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

When the amount of one or more biomarkers is measured, this amount can be compared to one or more reference levels (e.g., if one biomarker is measured (e.g., NF-L)), then one reference level will be needed (e.g., NF-L), if two biomarkers are measured (e.g., APOA1 and MBP), then two reference levels will be needed (e.g., one for APOA1 and one for MBP), if three biomarkers are measured (e.g., NSE, tau, hsCRP), then three reference levels will be needed (one for NSE, one for tau and one for hsCRP), etc. Generally, a reference level of one or more biomarkers can be employed as a benchmark against which to assess results obtained upon assaying a test sample for the one or more biomarkers. Generally, in making such a comparison, the reference level of the one or more biomarkers is obtained by running a particular assay a sufficient number of times and under appropriate conditions such that a linkage or association of analyte presence, amount or concentration with a particular stage or endpoint of TBI or with particular indicia can be made. Typically, the reference level of the one or more biomarkers is obtained with assays of reference subjects (or populations of subjects). The one or more biomarkers measured can include fragments thereof, degradation products thereof, and/or enzymatic cleavage products thereof.

In certain embodiments, the reference level may be correlated with control subjects that have not sustained a head injury.

In one aspect, a (preliminary) first TBI outcome diagnosis is made based on the amount of the one or more biomarkers determined (e.g., measured) by the assay and optionally, whether the amount of the one or more biomarkers have increased or decreased when compared to the one or more reference levels. For example, the (preliminary) first TBI outcome diagnosis may be based on the amount of the one or more biomarkers determined (e.g., measured) in one or more samples obtained from the subject (and optionally, whether they have increased or decreased when compared to one or more reference levels) in order to predict that: (1) the subject will likely have a favorable outcome from the TBI; or (2) the subject will likely not have a favorable outcome from the TBI. In another aspect, the amount (data) of the one or more biomarkers determined by the at least one assays can be used in a computer-implemented method to generate a single TBI outcome diagnosis as discussed in more detail below. In addition to performing the at least one assay on one or more samples taken from the subject, the method described herein further involves performing at least one advanced MRI procedure on the subject after a diagnosis of a TBI (such as within 24 hours after diagnosis of a TBI, within 48 hours of diagnosis of a TBI or within 72 hours of diagnosis of a TBI). The at least one advanced magnetic resonance procedure performed on the subject can be gradient echo magnetic resonance imaging, echo planar magnetic resonance imaging, spin echo magnetization resonance imaging, diffusion MRI (e.g., multi-shell diffusion MRI), resting-state functional MRI, magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof.

The advanced MRI performed generates data on the brain of the subject, such as, for example, diffusion MRI data (e.g, multi-shell diffusion MRI), resting-state functional MRI data, etc.

In one aspect, the data obtained from the at least one advanced MRI procedure can be used in a computer-implemented method to generate a (preliminary) second TBI outcome diagnosis for the subject. Examples of computer-implemented methods that can be used to generate the (preliminary) second TBI outcome diagnosis include those disclosed in U.S. Pat. No. 9,265,441, the contents of which are herein incorporated by reference. Alternatively, another example is READY View image analysis software available from GE Healthcare (GE Medical Systems SCS) (Buc, France) that allows users to process dynamic or functional volumetric data and to generate maps that display changes in image intensity over time, echo time, b-value and frequency, can also be used. For example, the advanced MRI techniques can provide information on the structural and functional connectivity of the subject's brain. However, any computer-implemented method that utilizes data obtained from at least one advanced MRI procedure can be used in the methods of the present invention.

Once the first and second TBI outcome diagnoses are generated, these diagnoses are combined to generate a final TBI outcome diagnosis of whether or not the subject will have a favorable or unfavorable outcome as a result of the TBI. Specifically, the first and second TBI outcome diagnoses can be combined to provide a final TBI outcome diagnosis that the subject will: (1) likely have a favorable outcome as a result of the TBI; or (2) not likely have a favorable outcome as a result of the TBI.

In another aspect, the amount of the one or more biomarkers determined (e.g., measured) by the assay and the data obtained from the at least one advanced MRI procedure can be used in a computer-implemented method to generate a single TBI outcome diagnosis for the subject. The TBI outcome diagnosis can be used to predict whether the subject will: (1) likely have a favorable outcome as a result of the TBI; or (2) not likely have a favorable outcome as a result of the TBI.

The at least one advanced MRI procedure can be performed on the subject either prior to or after the at least one assay is performed. In fact, the order in which the at least one assay or advanced MRI procedure are performed is not critical.

In some embodiments of the methods described herein, the sample may be obtained or taken from the subject about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours about 24 hours, within about 25 hours, with about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours or within about 72 hours after a TBI diagnosis.

In some embodiments, the subject has received a Glasgow Coma Scale score before or after the at least one assay, at least one advanced MRI, or the at least one assay and at least one advanced MRI is performed. In some embodiments, the subject is suspected as having moderate or severe traumatic brain injury or a moderate to severe traumatic brain injury based on the Glasgow Coma Scale score. In some embodiments, the reference level of at least one biomarker is correlated with subjects having moderate to severe traumatic brain injury. In some embodiments, the reference level of the at least one biomarker is correlated with a Glasgow Coma Scale score of 9-13 (a moderate TBI). In some embodiments, the reference level of the biomarker is correlated with a Glasgow Coma Scale score of 3-8 (a severe TBI). In some embodiments, the reference level of the biomarker is correlated with a Glasgow Coma Scale score of 3-12 (a moderate to severe TBI). In some embodiments, the subject is suspected as having mild traumatic brain injury based on the Glasgow Coma Scale score. In some embodiments, the reference level of the biomarker is correlated with subjects having mild traumatic brain injury. In some embodiments, the reference level of the biomarker is correlated with a Glasgow Coma Scale score of 13-15 (mild TBI).

In some embodiments, the methods described herein further include treating a human subject assessed as having a traumatic brain injury with a traumatic brain injury treatment, as described below. In yet other embodiments, the method further includes monitoring a subject assessed with a traumatic brain injury treatment. In some embodiments, the method further includes monitoring a human subject assessed as having a traumatic brain injury, as described below.

The nature of the assay employed in the methods described herein is not critical and the test can be any assay known in the art such as, for example, immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Nonetheless, tests or assays competent to perform the claimed methods will be employed, such as, for example, assays having various sensitivities and sensitivities as described herein. Moreover, the assays employed in the methods described herein can be employed in a clinical chemistry format such as would be known by one of ordinary skill in the art. Such assays are described in further detail herein in Section 8. It is known in the art that the values (e.g., reference levels, cutoffs, thresholds, specificities, sensitivities, concentrations of calibrators and/or controls etc.) used in an assay that employs specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4^{th} edition, chapter 3.5, pages 315-322, the contents of which are herein incorporated by reference).

### 5. Methods for Determining a Course of Treatment or Efficacy of a Course of Treatment for a Human Subject that has Suffered a TBI

The present disclosure relates, among other methods, to a method for determining a course of treatment or determining the efficacy of a course of treatment of a human subject that has been determined to have (or, more likely than not, have) suffered a traumatic brain injury, such as, for example, a mild, moderate, severe, or moderate to severe TBI. In some embodiments, the subject determined to have (or, more likely than not, have) suffered a TBI may not have received any prior treatment at the time the method is performed. In such instances, the method described herein can be used to determine or develop a course of treatment. In other embodiments of this method, the subject determined to have (or, more likely than not, have) suffered a traumatic brain injury has received or is receiving one or more courses of treatment for the TBI. In such instances, the method described herein can be used to determine the efficacy of such treatment and whether any alterations of the course of treatment are necessary.

The method described herein involves: (1) detecting or determining the presence or amount of one or more biomarkers in as sample obtained from the subject that has been diagnosed with a TBI; (2) obtaining data from the subject using at least one advanced magnetic resonance imaging procedure; and (3) processing all or part of the information and data obtained from the biomarkers and/or at least one advanced MRI using a computer-implemented method.

The method involves performing at least one assay on one or more samples (e.g., such as a first sample, a second sample, etc.) obtained from the human subject who has suffered a TBI to determine the presence of (e.g., detect) or measure a level of one or more biomarkers (for example, one biomarker, two biomarkers, three biomarkers, four biomarkers, five biomarkers, six biomarkers, seven biomarkers, eight biomarkers, nine biomarkers, ten biomarkers, eleven biomarkers, twelve biomarkers, thirteen biomarkers, fourteen biomarkers, fifteen biomarkers, sixteen biomarkers, seventeen biomarkers, eighteen biomarkers, nineteen biomarkers, twenty biomarkers, etc.) in the sample using routine techniques known in the art. If more than one sample is obtained from the subject, the sample can be obtained at various time points after the first sample was obtained (e.g., such as about 1 minute after the first or subsequent sample was taken, about 2 minutes after the first or subsequent sample was taken, about 3 minutes after the first or subsequent sample was taken, about 4 minutes after the first or subsequent sample was taken, about 5 minutes after the first or subsequent sample was taken, about 10 minutes after the first or subsequent sample was taken, about 30 minutes after the first or subsequent sample was taken, about 45 minutes after the first or subsequent sample was taken, about 1 hour after the first or subsequent sample was taken, about 2 hours after the first or subsequent sample was taken, about 3 hours after the first or subsequent sample was taken, about 4 hours after the first or subsequent sample was taken, about 5 hours after the first or subsequent sample was taken, about 6 hours after the first or subsequent sample was taken, about 7 hours after the first or subsequent sample was taken, about 8 hours after the first or subsequent sample was taken, about 9 hours after the first or subsequent sample was taken, about 10 hours after the first or subsequent sample was taken, about 11 hours after the first or subsequent sample was taken, about 12 hours after the first or subsequent sample was taken, etc.

Any biomarkers of TBI can be used in the methods described herein. Examples of such biomarkers include: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b). In another aspect, one or more biomarkers that can be used in the methods described herein include: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

When the amount of one or more biomarkers is measured, this amount can be compared to one or more reference levels (e.g., if one biomarker is measured (e.g., NF-L)), then one reference level will be needed (e.g., NF-L), if two biomarkers are measured (e.g., APOA1 and MBP), then two reference levels will be needed (e.g., one for APOA1 and one for MBP), if three biomarkers are measured (e.g., NSE, tau, hsCRP), then three reference levels will be needed (one for NSE, one for tau and one for hsCRP), etc. Generally, a reference level of one or more biomarkers can be employed as a benchmark against which to assess results obtained upon assaying a test sample for the one or more biomarkers. Generally, in making such a comparison, the reference level of the one or more biomarkers is obtained by running a particular assay a sufficient number of times and under appropriate conditions such that a linkage or association of analyte presence, amount or concentration with a particular stage or endpoint of TBI or with particular indicia can be made. Typically, the reference level of the one or more biomarkers is obtained with assays of reference subjects (or populations of subjects). The one or more biomarkers measured can include fragments thereof, degradation products thereof, and/or enzymatic cleavage products thereof.

In certain embodiments, the reference level may be correlated with control subjects that have not sustained a head injury.

In one aspect, a (preliminary) first TBI treatment protocol can is based upon the amount of the one or more biomarkers determined (e.g., measured) by the assay and optionally, whether the amount of the one or more biomarkers have increased or decreased when compared to one or more reference levels. For example, the (preliminary) first TBI treatment protocol can be based on the amount of the one or more biomarkers determined (e.g., measured) in the one or more samples obtained from the in the subject (and optionally, whether they have increased or decreased when compared to one or more reference levels) indicates that: (1) the subject diagnosed with a TBI who was previously not receiving any course of treatment, should begin a course of treatment, such as one of the courses of treatment described herein in Section 5; or (2) the subject diagnosed with a TBI and previously receiving a course of treatment should (i) continue the existing course of treatment; (i) discontinue the existing course of treatment and institute a different type of treatment; or (iii) add one or more additional treatments to the existing treatment.

In another aspect, the amount (data) of the one or more biomarkers determined (e.g., measured) by the at least one assays can be used in a computer-implemented method to generate a single TBI treatment protocol as discussed in more detail below. In addition to performing the at least one assay on one or more samples taken from the subject, the method described herein further involves performing at least one advanced MRI procedure on the subject. The at least one advanced magnetic resonance procedure performed on the subject can be gradient echo magnetic resonance imaging, echo planar magnetic resonance imaging, spin echo magnetization resonance imaging, diffusion MRI (e.g., multi-shell diffusion MRI), resting-state functional MRI, magnetic resonance spectroscopy, perfusion magnetic resonance imaging (e.g., such as arterial spin labeling (ASL) perfusion), dynamic susceptibility weighted dynamic susceptibility MRI, dynamic contrast-enhanced MRI, quantitative structural MRI, magnetization transfer MRI or combinations thereof. The performance of the advanced MRI generates data on the brain of the subject, such as, for example, diffusion MRI data, resting-state functional MRI data, etc.

In one aspect, the data obtained from the at least one advanced MRI procedure can be used in a computer-implemented method to generate a (preliminary) second TBI treatment protocol for the subject. Examples of computer-implemented methods that can be used to generate the (preliminary) second TBI diagnosis include those disclosed in U.S. Pat. No. 9,265,441, the contents of which are herein incorporated by reference. Alternatively, another example is READY View image analysis software available from GE Healthcare (GE Medical Systems SCS) (Buc, France) that allows users to process dynamic or functional volumetric data and to generate maps that display changes in image intensity over time, echo time, b-value and frequency, can also be used. For example, the advanced MRI techniques can provide information on the structural and functional connectivity of the subject's brain. However, any computer-implemented method which utilizes data obtained from at least one advanced MRI procedure can be used in the methods described herein.

Once the first and second TBI treatment protocols are generated, these protocols are combined to generate a final TBI treatment protocol to determine, design or develop: (1) a course of treatment for the subject (e.g., such as in circumstances where the subject was not previously receiving any treatment); (2) whether an existing course of treatment is efficacious for a subject; or (3) whether a course of treatment needs to be altered, discontinued and/or supplemented.

In another aspect, the amount of the one or more biomarkers determined (e.g., measured) by the assay and the data obtained from the at least one advanced MRI procedure can be used in a computer-implemented method to generate a single TBI treatment protocol for the subject. The TBI treatment protocol can be used to determine, design or develop: (1) a course of treatment for the subject (e.g., such as in circumstances where the subject was not previously receiving any treatment); (2) whether an existing course of treatment is efficacious for a subject; or (3) whether a course of treatment needs to be altered, discontinued and/or supplemented.

The at least one advanced MRI procedure can be performed on the subject either prior to or after the at least one assay is performed. In fact, the order in which the at least one assay or advanced MRI procedure are performed is not critical.

In some embodiments, the subject has received a Glasgow Coma Scale score before or after the at least one assay, at least one advanced MRI, or the at least one assay and at least one advanced MRI is performed. In some embodiments, the subject is suspected as having moderate or severe traumatic brain injury or a moderate to severe traumatic brain injury based on the Glasgow Coma Scale score. In some embodiments, the reference level of at least one biomarker is correlated with subjects having moderate to severe traumatic brain injury. In some embodiments, the reference level of the at least one biomarker is correlated with a Glasgow Coma Scale score of 9-13 (a moderate TBI). In some embodiments, the reference level of the biomarker is correlated with a Glasgow Coma Scale score of 3-8 (a severe TBI). In some embodiments, the reference level of the biomarker is correlated with a Glasgow Coma Scale score of 3-12 (a moderate to severe TBI). In some embodiments, the subject is suspected as having mild traumatic brain injury based on the Glasgow Coma Scale score. In some embodiments, the reference level of the biomarker is correlated with subjects having mild traumatic brain injury. In some embodiments, the reference level of the biomarker is correlated with a Glasgow Coma Scale score of 13-15 (mild TBI).

In another aspect, in subjects identified as suffering from a traumatic brain injury (e.g., such as a mild, moderate, severe, or moderate to severe TBI), the amount of the one or more biomarkers determined by the assay and the data obtained from the at least one advanced MRI procedure can be used in a computer-implemented method to assess (i) the pathophysiology of the head injury (e.g., whether the subject has one or more of a cerebral hypofusion or hyperfusion, cerebral ischaemia, hyperaemia, impaired cerebrovascular CO₂-reactivity (such as, for example, cerebrovascular constriction or dilation in response to hypo- or hypercapnia), a cerebral vasospasm, brain tissue hypoxia, oedema (such as vasogenic brain or cytotoxic brain oedema), oxidative stress, cell death (such as, for example, necrosis or apoptosis) or any combinations thereof, the final determination can identify or characterize the subject as having or suffering or at risk of suffering from one or more of cerebral hypofusion or hyperfusion, cerebral ischaemia, hyperaemia, impaired cerebrovascular CO₂-reactivity (such as, for example, cerebrovascular constriction or dilation in response to hypo- or hypercapnia), a cerebral vasospasm, brain tissue hypoxia, oedema (such as vasogenic brain or cytotoxic brain oedema), oxidative stress, cell death (such as, for example, necrosis or apoptosis) or any combinations thereof); (ii) the endotype of the head injury (e.g., identifying that the injury is axonal, neuronal or astroglial, whether there is microvascular injury, whether there is proteinopathy, is there inflammation or any combinations thereof); or (iii) any combinations of (i)-(ii). Information on the pathophysiology of the head injury and/or the endotype of the head injury and optionally, the type of TBI (mild, moderate, severe, or moderate to severe) can be used to assess the degree of impairment in one or more systems of the brain and to determine, design or develop: (1) a course of treatment for the subject (e.g., such as in circumstances where the subject was not previously receiving any treatment); (2) whether an existing course of treatment is efficacious for a subject; or (3) whether a course of treatment needs to be altered, discontinued and/or supplemented.

In some embodiments, the method further includes treating a human subject assessed as having a traumatic brain injury with a traumatic brain injury treatment, as described below. In yet other embodiments, the method further includes monitoring a subject assessed with a traumatic brain injury with a traumatic brain injury treatment. In some embodiments, the method further includes monitoring a human subject assessed as having traumatic brain injury, as described below.

The nature of the assay employed in the methods described herein is not critical and the test can be any assay known in the art such as, for example, immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Nonetheless, tests or assays competent to perform the claimed methods will be employed, such as, for example, assays having various sensitivities and sensitivities as described herein. Moreover, the assays employed in the methods described herein can be employed in a clinical chemistry format such as would be known by one of ordinary skill in the art. Such assays are described in further detail herein in Sections 8. It is known in the art that the values (e.g., reference levels, cutoffs, thresholds, specificities, sensitivities, concentrations of calibrators and/or controls etc.) used in an assay that employs specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4^{th} edition, chapter 3.5, pages 315-322, the contents of which are herein incorporated by reference).

### 6. Treatment and Monitoring of Subjects Who have Sustained an Injury to the Head

The subject identified in the methods described above may be treated or monitored. In some embodiments, the method further includes treating the human subject with a traumatic brain injury treatment, such as any treatments known in the art. For example, treatment of traumatic brain injury can take a variety of forms depending on the severity of the injury to the head. For example, for some subjects the treatment may include one or more of rest, abstaining from physical activities, such as sports, avoiding light or wearing sunglasses when out in the light, medication for relief of a headache or migraine, anti-nausea medication, etc. Alternatively, for some subject, treatment may include administration of one or more appropriate medications (such as, for example, diuretics, anti-convulsant medications, medications to sedate and put an individual in a drug-induced coma, or other pharmaceutical or biopharmaceutical medications (either known or developed in the future for treatment of TBI), one or more surgical procedures (such as, for example, removal of a hematoma, repairing a skull fracture, decompressive craniectomy, etc.), protecting the airway, and one or more therapies (such as, for example one or more rehabilitation, cognitive behavioral therapy, anger management, counseling psychology, etc.). In some embodiments, the subject being treated has a mild TBI. In other aspects, the subject being treated has a moderate TBI. In yet other aspects, the subject being treated has a severe TBI. In yet other aspects, the subject being treated has a moderate to severe TBI. In yet further aspects, the method further includes monitoring the human subject (e.g., such as a subject with a mild TBI, a moderate TBI, a severe TBI, or a moderate to severe TBI). In some embodiments, a subject may be monitored with CT scan or MRI.

### 7. Methods for Measuring the Level of Biomarkers and Autoantibodies to Biomarkers

A. Biomarkers

The present disclosure involves assays to measure and/or detect a level of one or more biomarkers in one or more test samples obtained from a subject. Specifically, the one or more biomarkers that can be used in the methods described herein are any brain biomarkers. Examples of brain biomarkers which provide a measure or indication of traumatic brain injury include one or more biomarkers of neuronal or axonal injury, one or more biomarkers of astroglial injury, one or more biomarkers of neuroinflammation, one or more genetic biomarkers, autoantibodies to any of the foregoing and any combinations thereof. In one aspect, one or more biomarkers that can be used in the methods described herein include a) APOA1, CRP, NSE, BDNF, tau, p-tau, NF-L, S100β, UCH-L1, GFAP and combinations thereof; b) one or more autoantibodies to any of the biomarkers listed in a) or c) combinations of a) and b).

### 1. Ubiquitin Carboxy-Terminal Hydrolase L1 (UCH-L1)

Ubiquitin carboxy-terminal hydrolase L1 (“UCH-L1”), which is also known as “ubiquitin C-terminal hydrolase,” is a deubiquitinating enzyme. UCH-L1 is a member of a gene family whose products hydrolyze small C-terminal adducts of ubiquitin to generate the ubiquitin monomer. Expression of UCH-L1 is highly specific to neurons and to cells of the diffuse neuroendocrine system and their tumors. It is abundantly present in all neurons (accounts for 1-2% of total brain protein), expressed specifically in neurons and testis/ovary. The catalytic triad of UCH-L1 contains a cysteine at position 90, an aspartate at position 176, and a histidine at position 161 that are responsible for its hydrolase activity.

Human UCH-L1 may have the following amino acid sequence:

(SEQ ID NO: 1)
MQLKPMEINPEMLNKVLSRLGVAGQWREVDVLGLEEESLGSVPAPACALL
LLFPLTAQHENFRKKQIEELKGQEVSPKVYFMKQTIGNSCGTIGLIHAVA
NNQDKLGFEDGSVLKQFLSETEKMSPEDRAKCFEKNEAIQAAHDAVAQEG
QCRVDDKVNFHFILFNNVDGHLYELDGRMPFPVNHGASSEDTLLKDAAKV
CREFTEREQGEVRFSAVALCKAA.

The human UCH-L1 may be a fragment or variant of SEQ ID NO: 1. The fragment of UCH-L1 may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise a contiguous number of amino acids from SEQ ID NO: 1.

### 2. Glial Fibrillary Acidic Protein (GFAP)

Glial fibrillary acidic protein (GFAP) is a 50 kDa intracytoplasmic filamentous protein that constitutes a portion of the cytoskeleton in astrocytes, and it has proved to be the most specific marker for cells of astrocytic origin. GFAP protein is encoded by the GFAP gene in humans. GFAP is the principal intermediate filament of mature astrocytes. In the central rod domain of the molecule, GFAP shares considerable structural homology with the other intermediate filaments. GFAP is involved in astrocyte motility and shape by providing structural stability to astrocytic processes. Glial fibrillary acidic protein and its breakdown products (GFAP-BDP) are brain-specific proteins released into the blood as part of the pathophysiological response after traumatic brain injury (TBI). Following injury to the human CNS caused by trauma, genetic disorders, or chemicals, astrocytes proliferate and show extensive hypertrophy of the cell body and processes, and GFAP is markedly upregulated. In contrast, with increasing astrocyte malignancy, there is a progressive loss of GFAP production. GFAP can also be detected in Schwann cells, enteric glia cells, salivary gland neoplasms, metastasizing renal carcinomas, epiglottic cartilage, pituicytes, immature oligodendrocytes, papillary meningiomas, and myoepithelial cells of the breast.

Human GFAP may have the following amino acid sequence:

(SEQ ID NO: 2)
MERRRITSAARRSYVSSGEMMVGGLAPGRRLGPGTRLSLARMPPPLPTRV
DFSLAGALNAGFKETRASERAEMMELNDRFASYIEKVRFLEQQNKALAAE
LNQLRAKEPTKLADVYQAELRELRLRLDQLTANSARLEVERDNLAQDLAT
VRQKLQDETNLRLEAENNLAAYRQEADEATLARLDLERKIESLEEEIRFL
RKIHEEEVRELQEQLARQQVHVELDVAKPDLTAALKEIRTQYEAMASSNM
HEAEEWYRSKFADLTDAAARNAELLRQAKHEANDYRRQLQSLTCDLESLR
GTNESLERQMREQEERHVREAASYQEALARLEEEGQSLKDEMARHLQEYQ
DLLNVKLALDIEIATYRKLLEGEENRITIPVQTFSNLQIRETSLDTKSVS
EGHLKRNIVVKTVEMRDGEVIKESKQEHKDVM.

The human GFAP may be a fragment or variant of SEQ ID NO: 2. The fragment of GFAP may be between 5 and 400 amino acids, between 10 and 400 amino acids, between 50 and 400 amino acids, between 60 and 400 amino acids, between 65 and 400 amino acids, between 100 and 400 amino acids, between 150 and 400 amino acids, between 100 and 300 amino acids, or between 200 and 300 amino acids in length. The fragment may comprise a contiguous number of amino acids from SEQ ID NO: 2. The human GFAP fragment or variant of SEQ ID NO: 21 may be a GFAP breakdown product (BDP). The GFAP BDP may be 38 kDa, 42 kDa, 47 kDa, 25 kDa, 19 kDa, or 20 kDa.

### 3. Apolipoprotein A1

Apolipoprotein A1, also referred to as “APOA1,” is the major protein component of high density lipoprotein (HDL) in plasma. A preproprotein is proteolytically processed to generate the mature Apo A1 protein, which promotes cholesterol efflux from tissues to the liver for excretion, and is a cofactor for lecithin cholesterolacyltransferase (LCAT), an enzyme responsible for the formation of most plasma cholesteryl esters. This gene is closely linked with two other apolipoprotein genes on chromosome 11. Defects in this gene are associated with HDL deficiencies, including Tangier disease, and with systemic non-neuropathic amyloidosis. Variation in the apolipoprotein A1 gene (APOA1) might influence the function of the protein, and thus brain cholesterol metabolism, leading to an increased risk for Alzheimer's disease (Volbach et al., Ann. Neurol., 58 (3): 436-41 (2005)).

Human apolipoprotein A1 may have the following amino acid sequence:

(SEQ ID NO: 3)
MKAAVLTLAVLFLTGSQARHFWQQDEPPQSPWDRVKDLATVYVDVLKDSG
RDYVSQFEGSALGKQLNLKLLDNWDSVTSTFSKLREQLGPVTQEFWDNLE
KETEGLRQEMSKDLEEVKAKVQPYLDDFQKKWQEEMELYRQKVEPLRAEL
QEGARQKLHELQEKLSPLGEEMRDRARAHVDALRTHLAPYSDELRQRLAA
RLEALKENGGARLAEYHAKATEHLSTLSEKAKPALEDLRQGLLPVLESFK
VSFLSALEEYTKKLNTQ.

The human apolipoprotein A1 may be a fragment or variant of SEQ ID NO: 3. The fragment of apolipoprotein A1 may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise any contiguous number of amino acids from SEQ ID NO: 3.

### 4. Free and Total Brain Derived Neurotrophic Factor (BDNF)

Brain derived neurotrophic factor (BDNF) is the most prevalent growth factor in the central nervous system (CNS), and is essential for the development of the CNS and for neuronal plasticity. Because BDNF plays a crucial role in development and plasticity of the brain, it is widely implicated in psychiatric diseases. BDNF is a member of the neurotrophin family of growth factors along with nerve growth factor (NGF); neurotrophins-3 (NT-3), NT4/5 and NT-6. BDNF is synthesized as a precursor protein known as prepro-BDNF that is cleaved into pro-BDNF, which can then be further cleaved into mature BDNF (Lessmann et al., Prog. Neurobiol., 69: 341-374 (2003)).

Pro-BDNF and mature BDNF have been shown to activate different intracellular signaling pathways. Pro-BDNF signals through the low-affinity neurotrophin receptor p75 that is believed to be involved in apoptosis. Mature BDNF signals through its high-affinity tropomyosin-related kinase B (TrkB) receptor. BDNF binding to TrkB induces its dimerization autophosphorylation of the receptor tyrosine kinase, leading to activation of intracellular signaling cascades, as well as augmentation of N-methyl-d-aspartate (NDMA) receptor currents. There are at least three signaling transduction pathways that BDNF-TrkB activation can regulate: the phospholipase C γ (PLC γ) pathway, which leads to activation of protein kinase C; the phosphatidylinositol 3-kinase (PI3K) pathway, which activates serine/threonine kinase AKT; and the mitogen-activated protein kinase (MAPK, or extracellular signal related kinase (ERK)) pathway, which activates several downstream effectors. Decreased levels of BDNF are associated with neurodegenerative diseases characterized by neuronal loss, such as Parkinson's disease, Alzheimer's disease, multiple sclerosis, and Huntington's disease (see, e.g., Bathina, S. and U. N. Das, Arch. Med. Sci. 11(6): 1164-1178 (2015); Binder, D. K. and H. E. Scharfman, Growth Factors, 22(3): 123-131 (2004); Autry, A. E. and L. M. Monteggia, Pharmacol. Rev., 64(2): 238-258 (2012)).

The methods described herein can be used to detect both free and/or total BNDF in a sample. “Free BNDF” refers to mature BDNF that is not bound to a Trk receptor, while “total BNDF” refers to free BNDF and Trk-bound BNDF.

Human BNDF may have the following amino acid sequence:

(SEQ ID NO: 4)
MTILFLTMVISYFGCMKAAPMKEANIRGQGGLAYPGVRTHGTLESVNGPK
AGSRGLTSLADTFEHVIEELDEDQKVRPNEENNKDADLYTSRVMLSSQVP
LEPPLLFLLEEYKNYLDAANMSMRVRRHSDPARRGELSVCDSISEWVTAA
DKKTAVDMSGGTVTVLEKVPVSKGQLKQYFYETKCNPMGYTKEGCRGIDK
RHWNSQCRTTQSYVRALTMDSKKRIGWRFIRIDTSCVCTLTIKRGR.

The human BDNF may be a fragment or variant of SEQ ID NO: 4. The fragment of BDNF may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise any contiguous number of amino acids from SEQ ID NO: 4.

### 5. C-Reactive Protein (CRP)

C-reactive protein (CRP) is an annular (ring-shaped), pentameric protein found in blood plasma, whose levels rise in response to inflammation. CRP is an acute-phase protein of hepatic origin that increases following interleukin-6 secretion by macrophages and T cells. CRP binds to lysophosphatidylcholine expressed on the surface of dead or dying cells (and some types of bacteria) in order to activate the complement system via C1q (Thompson et al., Structure, 7(2): 169-177 (1999). A normal level of CRP in human blood is less than 10 milligram per liter (mg/L). CRP testing typically is used to assess inflammation in humans, which can indicate infection, chronic inflammatory diseases, as well as heart disease risk. In particular, high levels of CRP (e.g., 10-40 mg/L) may indicate mild or active inflammation, bacterial infection, and/or viral infection. High levels of CRP also are associated with an increased risk of heart disease.

Human CRP may have the following amino acid sequence:

(SEQ ID NO: 5)
MEKLLCFLVLTSLSHAFGQTDMSRKAFVFPKESDTSYVSLKAPLTKPLKA
FTVCLHFYTELSSTRGYSIFSYATKRQDNEILIFWSKDIGYSFTVGGSEI
LFEVPEVTVAPVHICTSWESASGIVEFWVDGKPRVRKSLKKGYTVGAEAS
IILGQEQDSFGGNFEGSQSLVGDIGNVNMWDFVLSPDEINTIYLGGPFSP
NVLNWRALKYEVQGEVFTKPQLWP.

The human CRP may be a fragment or variant of SEQ ID NO: 5. The fragment of CRP may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise any contiguous number of amino acids from SEQ ID NO: 5.

### 6. Neuron Specific Enolase (NSE)

Neuron specific enolase (NSE) is a 78 kD gamma-homodimer and represents the dominant enolase-isoenzyme found in neuronal and neuroendocrine tissues. Vertebrates express three isozymes of enolase: enolase α is ubiquitous; enolase β is muscle-specific, and enolase γ is neuron-specific. The expression of NSE, which occurs as γγ- and αγ-dimer, is a late event in neural differentiation, thus making it a useful index of neural maturation. NSE is a highly specific marker for neurons and peripheral neuroendocrine cells. Increased body fluids levels of NSE may occur with malignant proliferation, and thus can be used in diagnosis, staging, and treatment of related neuroendocrine tumor (NETs). NSE also is currently the most reliable tumor marker in diagnosis, prognosis, and follow-up of small cell lung cancer (SCLC). The level of NSE correlates with tumor burden, number of metastatic sites, and response to treatment. NSE can be also useful at diagnosis of NETs and gastroenteropancreatic (GEP)-NETs. Raised serum levels of NSE have been found in all stages of neuroblastoma, although the incidence of increased concentration is greater in widespread and metastatic disease. Moreover, NSE determination in cord blood offers an early postnatal possibility of confirming the diagnosis of neuroblastoma in newborns. NSE also may provide quantitative measures of brain damage and/or improve the diagnosis and the outcome evaluation in ischemic stroke, intracerebral hemorrhage, seizures, comatose patients after cardiopulmonary resuscitation for cardiac arrest, and traumatic brain injury. Increased NSE serum levels have also been found associated with melanoma, seminoma, renal cell carcinoma, Merkel cell tumor, carcinoid tumors, dysgerminomas and immature teratomas, malignant phaechromocytoma, Guillain-Barré syndrome and Creutzfeldt-Jakob disease (Isgro et al., Adv Exp Med Biol., 867: 125-43 (2015)).

Human neuron specific enolase may have the following amino acid sequence:

(SEQ ID NO: 6)
MSIEKIWAREILDSRGNPTVEVDLYTAKGLFRAAVPSGASTGIYEALELR
DGDKQRYLGKGVLKAVDHINSTIAPALISSGLSVVEQEKLDNLMLELDGT
ENKSKFGANAILGVSLAVCKAGAAERELPLYRHIAQLAGNSDLILPVPAF
NVINGGSHAGNKLAMQEFMILPVGAESFRDAMRLGAEVYHTLKGVIKDKY
GKDATNVGDEGGFAPNILENSEALELVKEAIDKAGYTEKIVIGMDVAASE
FYRDGKYDLDFKSPTDPSRYITGDQLGALYQDFVRDYPVVSIEDPFDQDD
WAAWSKFTANVGIQIVGDDLTVTNPKRIERAVEEKACNCLLLKVNQIGSV
TEAIQACKLAQENGWGVMVSHRSGETEDTFIADLVVGLCTGQIKTGAPCR
SERLAKYNQLMRIEEELGDEARFAGHNFRNPSVL.

The human NSE may be a fragment or variant of SEQ ID NO: 6. The fragment of NSE may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise any contiguous number of amino acids from SEQ ID NO: 6.

### 7. Neurofilament Light Protein (NF-L)

Neurofilament light protein (NF-L) is the smallest of three subunits that make up neurofilaments, which are major components of the neuronal cytoskeleton. NF-L is released from damaged neurons, and concentrations of NF-L in CSF are increased in people with neurodegenerative diseases, including Alzheimer's disease, amyotrophic lateral sclerosis, and frontotemporal dementia (Rosengren et al., Neurochem., 67: 2013-2018 (1996); and Landqvist et al., BMC Neurol., 13: 54 (2013)). Small-scale cross-sectional studies also found increased concentrations of NF-L in the CSF of individuals with Huntington's disease (Wild et al., J Clin Invest., 125: 1979-1986 (2015); Constantinescu et al., Parkinsonism Relat Disord., 15: 245-248 (2009); Vinther-Jensen et al., Neurol Neuroimmunol Neuroinflamm., 3: e287 (2016); and Niemela et al., PLoS One; 12: e0172762 (2017)).

Human neurofilament light protein may have the following amino acid sequence:

(SEQ ID NO: 7)
MSSFSYEPYYSTSYKRRYVETPRVHISSVRSGYSTARSAYSSYSAPVSSS
LSVRRSYSSSSGSLMPSLENLDLSQVAAISNDLKSIRTQEKAQLQDLNDR
FASFIERVHELEQQNKVLEAELLVLRQKHSEPSRFRALYEQEIRDLRLAA
EDATNEKQALQGEREGLEETLRNLQARYEEEVLSREDAEGRLMEARKGAD
EAALARAELEKRIDSLMDEISFLKKVHEEEIAELQAQIQYAQISVEMDVT
KPDLSAALKDIRAQYEKLAAKNMQNAEEWFKSRFTVLTESAAKNTDAVRA
AKDEVSESRRLLKAKTLEIEACRGMNEALEKQLQELEDKQNADISAMQDT
INKLENELRTTKSEMARYLKEYQDLLNVKMALDIEIAAYRKLLEGEETRL
SFTSVGSITSGYSQSSQVFGRSAYGGLQTSSYLMSTRSFPSYYTSHVQEE
QIEVEETIEAAKAEEAKDEPPSEGEAEEEEKDKEEAEEEEAAEEEEAAKE
ESEEAKEEEEGGEGEEGEETKEAEEEEKKVEGAGEEQAAKKKD.

The human NF-L protein may be a fragment or variant of SEQ ID NO: 7. The fragment of NF-L may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise any contiguous number of amino acids from SEQ ID NO: 7.

### 8. S100 Calcium-Binding Protein B (S100β)

S100 proteins are localized in the cytoplasm and nucleus of a wide range of cells, and involved in the regulation of a number of cellular processes such as cell cycle progression and differentiation. S100 calcium-binding protein B (S100β or S100β) is glial-specific and expressed primarily by astrocytes, but not all astrocytes express S100β. It has been shown that S100β is only expressed by a subtype of mature astrocytes that ensheath blood vessels, and by NG2-expressing cells (Wang and Bordey, Prog. Neurobiol., 86 (4): 342-67 (2008)). S100β may function in neurite extension, proliferation of melanoma cells, stimulation of Ca2+ fluxes, inhibition of PKC-mediated phosphorylation, astrocytosis and axonal proliferation, and inhibition of microtubule assembly. In the developing CNS, S100β acts as a neurotrophic factor and neuronal survival protein.

Chromosomal rearrangements and altered expression of the gene encoding S100β have been implicated in neurological, neoplastic, and other diseases, including Alzheimer's disease, Down's syndrome, epilepsy, amyotrophic lateral sclerosis, schwannoma, melanoma, and type I diabetes. S100β also has emerged as a candidate peripheral biomarker of blood-brain barrier (BBB) permeability and CNS injury. Elevated S100β levels may reflect the presence of neuropathological conditions including traumatic head injury or neurodegenerative diseases, while normal S100β levels reliably exclude major CNS pathology.

Human S100β may have the following amino acid sequence:

(SEQ ID NO: 8)
MSELEKAMVALIDVFHQYSGREGDKHKLKKSELKELINNELSHFLEEIKE
QEVVDKVMETLDNDGDGECDFQEFMAFVAMVTTACHEFFEHE.

The human S100β may be a fragment or variant of SEQ ID NO: 8. The fragment of S100β may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise any contiguous number of amino acids from SEQ ID NO: 8.

### 9. Tau and Phosphorylated Tau (p-Tau)

One of the typical brain lesions of Alzheimer's disease (AD) is neurofibrillary tangles (NFTs) that consist of hyperphosphorylated forms of the microtubule associated protein tau that is assembled into paired helical filaments or PHFs (Grundke-Iqbal et al., Proc Natl Acad Sci USA, 83: 4913-4917 (1986); Kosik et al., Proc Natl Acad Sci USA, 83: 4044-4048 (1986); Lee et al., Science, 251: 675-678 (1991); Nukina and Ihara, J. Biochem., 99: 1541-1544 (1986); Wood et al., Proc Natl Acad Sci USA, 83: 4040-4043 (1986)). Tau expression is high in non-myelinated cortical axons, especially in the regions of the brain that are involved in memory consolidation such as the limbic cortex including the hippocampus. Hyperphosphorylation of tau causes the protein to detach from the microtubules, thereby destabilizing microtubules and compromising axonal transport (Bramblett et al., Neuron, 10: 1089-1099 (1993); Ishihara et al., Neuron, 24:751-762 (1999)). While tau phosphorylation promotes axonal and synaptic plasticity in the developing brain (Lovestone and Reynolds, Neuroscience, 78:309-324 (1997)), it is pathological in the adult brain and specifically related to a group of disorders referred to as tauopathies, which includes AD and some forms of frontotemporal dementia (FTD) (Ballatore et al., Nat Rev Neurosci., 8: 663-672 (2007)). Tau proteins may be considered promising candidate biomarkers for Alzheimer-type axonal degeneration and NFT formation (Hampel et al., Experimental Gerontology, 45(1):30. doi:10.1016/j.exger.2009.10.010 (2010)).

Human tau protein and p-tau protein may have the following amino acid sequence:

(SEQ ID NO: 9)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQEPESGKVVQEGFLREPGPPGLSHQLM
SGMPGAPLLPEGPREATRQPSGTGPEDTEGGRHAPELLKHQLLGDLHQEG
PPLKGAGGKERPGSKEEVDEDRDVDESSPQDSPPSKASPAQDGRPPQTAA
REATSIPGFPAEGAIPLPVDFLSKVSTEIPASEPDGPSVGRAKGQDAPLE
FTFHVEITPNVQKEQAHSEEHLGRAAFPGAPGEGPEARGPSLGEDTKEAD
LPEPSEKQPAAAPRGKPVSRVPQLKARMVSKSKDGTGSDDKKAKTSTRSS
AKTLKNRPCLSPKHPTPGSSDPLIQPSSPAVCPEPPSSPKYVSSVTSRTG
SSGAKEMKLKGADGKTKIATPRGAAPPGQKGQANATRIPAKTPPAPKTPP
SSGEPPKSGDRSGYSSPGSPGTPGSRSRTPSLPTPPTREPKKVAVVRTPP
KSPSSAKSRLQTAPVPMPDLKNVKSKIGSTENLKHQPGGGKVQIINKKLD
LSNVQSKCGSKDNIKHVPGGGSVQIVYKPVDLSKVTSKCGSLGNIHHKPG
GGQVEVKSEKLDFKDRVQSKIGSLDNITHVPGGGNKKIETHKLTFRENAK
AKTDHGAEIVYKSPVVSGDTSPRHLSNVSSTGSIDMVDSPQLATLADEVS
ASLAKQGL

The human tau or p-tau protein may be a fragment or variant of SEQ ID NO: 9. The fragment of tau or p-tau may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise any contiguous number of amino acids from SEQ ID NO: 9.

B. Measuring the Level of Biomarkers

In the methods described above, the levels of one more biomarkers disclosed herein can be measured by any means, such as antibody dependent methods, such as immunoassays, protein immunoprecipitation, immunoelectrophoresis, chemical analysis, SDS-PAGE and Western blot analysis, protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in clinical chemistry format such as would be known by one skilled in the art.

In some embodiments, measuring the level of one or more biomarkers includes contacting the sample with a first specific binding member and second specific binding member. In some embodiments the first specific binding member is a capture antibody and the second specific binding member is a detection antibody. In some embodiments, measuring the level of one or more biomarkers includes contacting the sample, either simultaneously or sequentially, in any order with: (1) a capture antibody (e.g., a biomarker-capture antibody), which binds to an epitope on the biomarker or biomarker fragment to form a capture antibody-biomarker antigen complex (e.g., a biomarker-capture antibody-biomarker antigen complex), and (2) a detection antibody (e.g., a biomarker-detection antibody), which includes a detectable label and binds to an epitope on the biomarker that is not bound by the capture antibody, to form a biomarker antigen-detection antibody complex (e.g., a biomarker antigen-a biomarker-detection antibody complex), such that a capture antibody-biomarker antigen-detection antibody complex (e.g., a biomarker-capture antibody-biomarker antigen-biomarker-detection antibody complex) is formed, and measuring the amount or concentration of a biomarker in the sample based on the signal generated by the detectable label in the capture antibody-biomarker antigen-detection antibody complex.

In some embodiments, the first specific binding member is immobilized on a solid support. In some embodiments, the second specific binding member is immobilized on a solid support. In some embodiments, the first specific binding member is a biomarker antibody as described below.

In some embodiments, the sample is diluted or undiluted.

Some instruments (such as, for example the Abbott Laboratories instrument ARCHITECT®, and other core laboratory instruments) other than a point-of-care device may be capable of measuring levels of one or more biomarkers in a sample higher or greater than 25,000 pg/mL.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety.

### 1. Biomarker-Recognizing Antibodies

The methods described herein may use an isolated antibody that specifically binds to one or more biomarkers, such as, one or more biomarkers of neuronal or axonal injury, one or more biomarkers of astroglial injury, one or more biomarkers of neuroinflammation, one or more genetic biomarkers, and any combinations thereof. Such an antibody may be referred to as a “biomarker antibody,” an “anti-biomarker antibody,” “APOA1 antibody,” “a BDNF antibody,” “a NSE antibody,” “a NF-L antibody,” “a S100β antibody”, a “NF-L antibody”, a “CRP antibody” and the like. The biomarker antibodies can be used to assess the biomarker status as a measure of traumatic brain injury, detect the presence of a biomarker in a sample, quantify the amount of biomarker present in a sample, or detect the presence of and quantify the amount of one or more biomarkers in a sample.

The antibody is an antibody that binds to one or more biomarkers, a fragment thereof, an epitope of one or more biomarkers, or a variant thereof. The antibody may be a fragment of an anti-biomarker antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

An anti-biomarker antibody may be a chimeric anti-biomarker or humanized anti-biomarker antibody. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008(8):85. Therefore, the antibody may be a product of the human and not animal repertoire. Because it is of human origin, the risks of reactivity against self-antigens may be minimized. Alternatively, standard yeast display libraries and display technologies may be used to select and isolate human anti-biomarker antibodies. For example, libraries of naïve human single chain variable fragments (scFv) may be used to select human anti-biomarker antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

### 2. Biomarker Epitope(s)

The antibody may immunospecifically bind to a biomarker (such as any of those described herein), a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region.

### 3. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, in order to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77: 4216-4220 (1980)), used with a DHFR selectable marker, e.g., as described in Kaufman and Sharp, J. Mol. Biol., 159: 601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., an antibody that binds human AMPAR) and the other heavy and light chain are specific for an antigen other than human AMPAR by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods for preparing monoclonal antibodies involve the preparation of immortal cell lines capable of producing antibodies having the desired specificity. Such cell lines may be produced from spleen cells obtained from an immunized animal. The animal may be immunized with a biomarker or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH::VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art (see, e.g., U.S. Pat. Nos. 4,704,692; 5,723,323; 5,763,192; 5,814,476; 5,817,483; 5,824,514; and 5,976,862). Alternative methods rely upon immunization of transgenic animals (e.g., SCID mice, Nguyen et al. (1997) Microbiol. Immunol., 41:901-907; Sandhu et al. (1996) Crit. Rev. Biotechnol., 16:95-118; Eren et al. (1998) Immunol., 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol., 8:333-337; One Cell Systems, (Cambridge, Mass.); Gray et al. (1995) J. Imm. Meth., 182:155-163; Kenny et al. (1995) Bio/Technol. 13:787-790); and B-cell selection (Steenbakkers et al. (1994) Molec. Biol. Reports 19:125-134 (1994)).

An affinity-matured antibody may be produced by any one of a number of procedures that are known in the art. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); and Hawkins et al, J. Mol. Biol., 226: 889-896 (1992). Selective mutation at selective mutagenesis positions and at contact or hypermutation positions with an activity enhancing amino acid residue is described in, for example, U.S. Pat. No. 6,914,128 B1.

Antibody variants can also be prepared by delivering a polynucleotide encoding an antibody to a suitable host such as to provide transgenic animals or mammals, such as goats, cows, horses, sheep, and the like, that produce such antibodies in their milk. These methods are known in the art and are described in, for example, U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants, cultured plant cells, and/or specified portions or variants of plant parts (e.g., but not limited to tobacco, maize, and duckweed) that produce such antibodies. For example, Cramer et al. (1999) Curr. Top. Microbiol. Immunol., 240:95-118, and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol., (1999) 464:127-147, and references cited therein. Antibody variants have also been produced in large amounts from transgenic plant seeds including antibody fragments, such as single chain antibodies (scFvs), including tobacco seeds and potato tubers. See, e.g., Conrad et al. (1998) Plant Mol. Biol., 38:101-109, and reference cited therein. Thus, antibodies can also be produced using transgenic plants according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al., (1995) Protein Eng., 8(10):1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (131I), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from Chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibody production via the use of hybridoma technology, the selected lymphocyte antibody method (SLAM), transgenic animals, and recombinant antibody libraries is described in more detail below.

(a) Production of Anti-Biomarker Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the invention wherein, preferably, the hybridoma is generated by fusing splenocytes isolated from an animal, e.g., a rat or a mouse, immunized with a biomarker antigen with myeloma cells and then screening the hybridomas resulting from the fusion for hybridoma clones that secrete an antibody able to bind a biomarker of the invention. Briefly, rats can be immunized with a biomarker antigen. In a preferred embodiment, the biomarker antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a biomarker antigen, antibodies and/or antibody-producing cells may be obtained from the animal. An anti-biomarker antibody-containing serum is obtained from the animal by bleeding or sacrificing the animal. The serum may be used as it is obtained from the animal, an immunoglobulin fraction may be obtained from the serum, or the anti-biomarker antibodies may be purified from the serum. Serum or immunoglobulins obtained in this manner are polyclonal, thus having a heterogeneous array of properties.

Once an immune response is detected, e.g., antibodies specific for the biomarker antigen are detected in the rat serum, the rat spleen is harvested, and splenocytes are isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding a biomarker. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using the biomarker, or a portion thereof, or a cell expressing the biomarker. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-biomarker antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-biomarker antibody.

Antibody fragments that recognize specific epitopes may be generated by known techniques. For example, Fab and F(ab′)2 fragments of the invention may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

(b) Production of Anti-Biomarker Monoclonal Antibodies Using SLAM

In another aspect of the invention, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93: 7843-7848 (1996). In this method, single cells secreting antibodies of interest, e.g., lymphocytes derived from any one of the immunized animals are screened using an antigen-specific hemolytic plaque assay, wherein the biomarker antigen, or a subunit or fragment thereof, is coupled to sheep red blood cells using a linker, such as biotin, and used to identify single cells that secrete antibodies with specificity for the biomarker. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to the biomarker. The amplified immunoglobulin sequences further can be manipulated in vitro, such as by in vitro affinity maturation method. See, for example, PCT Publication No. WO 97/29131 and PCT Publication No. WO 00/56772.

(c) Production of Anti-Biomarker Monoclonal Antibodies Using Transgenic Animals

In another embodiment of the invention, antibodies are produced by immunizing a non-human animal comprising some, or all, of the human immunoglobulin locus with a biomarker antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7: 13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies, and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15: 146-156 (1997), Green and Jakobovits, J. Exp. Med., 188: 483-495 (1998), the disclosures of which are hereby incorporated by reference.

(d) Production of Anti-Biomarker Monoclonal Antibodies Using Recombinant Antibody Libraries

In vitro methods also can be used to make the antibodies of the invention, wherein an antibody library is screened to identify an antibody having the desired biomarker-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9: 1369-1372 (1991); Hay et al., Hum. Antibod. Hybridomas, 3: 81-85 (1992); Huse et al., Science, 246: 1275-1281 (1989); McCafferty et al., Nature, 348: 552-554 (1990); Griffiths et al., EMBO J., 12: 725-734 (1993); Hawkins et al., J. Mol. Biol., 226: 889-896 (1992); Clackson et al., Nature, 352: 624-628 (1991); Gram et al., Proc. Natl. Acad. Sci. USA, 89: 3576-3580 (1992); Garrard et al., Bio/Technology, 9: 1373-1377 (1991); Hoogenboom et al., Nucl. Acids Res., 19: 4133-4137 (1991); Barbas et al., Proc. Natl. Acad. Sci. USA, 88: 7978-7982 (1991); U.S. Patent Application Publication No. 2003/0186374; and PCT Publication No. WO 97/29131, the contents of each of which are incorporated herein by reference.

The recombinant antibody library may be from a subject immunized with a biomarker, or a portion of a biomarker. Alternatively, the recombinant antibody library may be from a naive subject, e.g., one who has not been immunized with a biomarker, such as a human antibody library from a human subject who has not been immunized with a human biomarker. Antibodies of the invention are selected by screening the recombinant antibody library with the peptide comprising biomarker antigen to thereby select those antibodies that recognize the biomarker. Methods for conducting such screening and selection are well known in the art, such as described in the references in the preceding paragraph. To select antibodies of the invention having particular binding affinities for a biomarker, such as those that dissociate from a human biomarker with a particular K_{off} rate constant, the art-known method of surface plasmon resonance can be used to select antibodies having the desired K_{off} rate constant. To select antibodies of the invention having a particular neutralizing activity for a human biomarker, such as those with a particular IC₅₀, standard methods known in the art for assessing the inhibition of activity of a specific biomarker may be used.

In one aspect, the invention pertains to an isolated antibody, or an antigen-binding portion thereof, that binds a human biomarker. Preferably, the antibody is a neutralizing antibody. In various embodiments, the antibody is a recombinant antibody or a monoclonal antibody.

Antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182: 41-50 (1995); Ames et al., J. Immunol. Methods, 184:177-186 (1995); Kettleborough et al., Eur. J. Immunol., 24: 952-958 (1994); Persic et al., Gene, 187: 9-18 (1997); Burton et al., Advances in Immunology, 57: 191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

After phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12(6): 864-869 (1992); Sawai et al., Am. J. Reprod. Immunol., 34: 26-34 (1995); and Better et al., Science, 240: 1041-1043 (1988). Examples of techniques which can be used to produce single-chain Fvs and antibodies include those described in U.S. Pat. Nos. 4,946,778 and 5,258,498; Huston et al., Methods in Enzymology, 203: 46-88 (1991); Shu et al., Proc. Natl. Acad. Sci. USA, 90: 7995-7999 (1993); and Skerra et al., Science, 240: 1038-1041 (1988).

Alternative to screening of recombinant antibody libraries by phage display, other methodologies known in the art for screening large combinatorial libraries can be applied to the identification of antibodies of the invention. One type of alternative expression system is one in which the recombinant antibody library is expressed as RNA-protein fusions, as described in PCT Publication No. WO 98/31700 (Szostak and Roberts), and in Roberts and Szostak, Proc. Natd. Acad. Sci. USA, 94: 12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

(e) Production of Humanized Antibodies

A humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (e.g., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present invention can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for a biomarker and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristics, such as increased affinity for a biomarker, are achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g., mice that are capable, upon immunization, of producing a full repertoire of human antibodies in the absence of endogenous immunoglobulin production. For example, the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in, for example, U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

Anti-biomarker antibodies may be generated using any of the techniques described above as well as using routine techniques known in the art. For example, the anti-biomarker antibody may be an unconjugated biomarker antibody, such as anti-NSE or anti-S100β antibodies, available from United State Biological (Catalog Number: 031320), Cell Signaling Technology (Catalog Number: 3524), Sigma-Aldrich (Catalog Number: HPA005993), Santa Cruz Biotechnology, Inc. (Catalog Numbers: sc-58593 or sc-58594), R&D Systems (Catalog Number: MAB6007), Novus Biologicals (Catalog Number: NB600-1160), Biorbyt (Catalog Number: orb33715), Enzo Life Sciences, Inc. (Catalog Number: ADI-905-520-1), Bio-Rad (Catalog Number: VMA00004), BioVision (Catalog Number: 6130-50), Abcam (Catalog Numbers: ab75275 or ab104938), Invitrogen Antibodies (Catalog Numbers: 480012), ThermoFisher Scientific (Catalog Numbers: MA1-46079, MA5-17235, MA1-90008, or MA1-83428), EMD Millipore (Catalog Number: MABN48), or Sino Biological Inc. (Catalog Number: 50690-R011). The anti-AMPAR antibody may be conjugated to a fluorophore, such as conjugated AMPAR antibodies available from BioVision (Catalog Number: 6960-25) or Aviva Systems Biology (Cat. Nos. OAAF01904-FITC).

C. Measuring the Level of Brain Biomarker Autoantibodies

In the methods described above, brain biomarker autoantibody levels can be measured by any means known in the art such as protein microarrays, mass spectrometry-based techniques (including liquid chromatography coupled to tandem mass spectrometry (LC-MS/MS), nano LC-MS/MS, matrix-assisted laser desorption/ionization mass spectrometry (MALDI-MS) as described in WO 2009/004576 (including surface enhanced laser desorption/ionization mass spectrometry (SELDI-MS), especially surfaced-enhanced affinity capture (SEAC), surface-enhanced need desorption (SEND) or surface-enhanced phot label attachment and release (SEPAR), matrix-assisted laser desorption/ionization time-of-flight (MALDI-TOF) mass spectrometry, antigen based immunoassays, antibody based techniques (including antibody based immunoassays, Western blotting, immunoblotting, ELISA “sandwich” immunoassays, radioimmunoassay (RIA), immunoprecipitation and dissociation-enhanced lanthanide fluoro immuno assay (DELFIA), precipitation reactions, gel diffusion precipitin reactions, immunodiffusion assays, immunoradiometric assays and protein A immunoassays), proteomic techniques, surface plasmon resonance (SPR), versatile fibre-based SPR, chemiluminescence, fluorescent polarization, phosphorescence, immunohistochemistry, immunofluorescence, microcytometry, microscopy, fluorescence activated cell sorting (FACS) and flow cytometry.

In some embodiments, measuring the level of one or more brain biomarker autoantibodies includes contacting the sample with a first specific binding member and second specific binding member. In some embodiments the first specific binding member is a capture antigen and the second specific binding member is a detection antigen. In some embodiments, measuring the level of a brain biomarker includes contacting the sample, either simultaneously or sequentially, in any order: (1) a capture antigen (e.g., a brain biomarker-capture antigen), which binds to an brain biomarker autoantibody to form a capture antigen-brain biomarker autoantibody complex (e.g., a brain biomarker-capture antigen-brain biomarker autoantibody complex), and (2) a detection antigen (e.g., a brain biomarker-detection antigen), which includes a detectable label and binds to a different location (epitope) on the brain biomarker autoantibody that is not bound by the capture antigen, to form a brain biomarker autoantibody-detection antigen complex (e.g., a brain biomarker autoantibody-brain biomarker-detection antigen complex), such that a capture antigen-brain biomarker autoantibody-detection antigen complex (e.g., a brain biomarker-capture antigen-brain biomarker autoantibody-brain biomarker-detection antigen complex) is formed, and measuring the amount or concentration of brain biomarker autoantibody in the sample based on the signal generated by the detectable label in the capture antigen-brain biomarker autoantigen-detection antigen complex.

In some embodiments, the first specific binding member is immobilized on a solid support. In some embodiments, the second specific binding member is immobilized on a solid support. In some embodiments, the first specific binding member, second specific binding member or both the first specific binding member and second specific binding member is a brain biomarker antigen. The brain biomarker can be a full-length brain biomarker protein or a fragment thereof, provided that the fragment is recognized by the autoantibody. For example, the fragment of the brain biomarker antigen may consist of as little as 5 amino acid residues of the antigen. In other aspect, the brain biomarker antigen may consist of as little as about 6 amino acid residues, at least 7 amino acid residues, at least 8 amino acid residues, at least 9 amino acid residues, at least 10 amino acid residues, at least 15 amino acid residues, at least 20 amino acid residues, at least 25 amino acid residues, at least 30 amino acid residues, at least 35 amino acid residues, at least 40 amino acid residues or at least 50 amino acid residues of the brain biomarker antigen. It is preferred that the fragment of the brain biomarker antigen represents an epitope sequence which can be recognized by the brain biomarker autoantibody. The size or type of the epitope is not limited as long as it can be recognized by the autoantibody.

Methods of making brain biomarker antigens are well known in the art. For example, recombinant DNA techniques may be used wherein a DNA molecule encoding a brain biomarker antigen, or part thereof, can be genetically engineered into an appropriate expression vector for large scale preparation of the brain biomarker antigen. It may also be advantageous to engineer fusion proteins that can facilitate labelling, immobilization or detection of the brain biomarker antigen. Many methods are also disclosed in standard molecular biology textbooks such as Green M. R. and Sambrook, J., Molecular Cloning: A Laboratory Manual (4^{th} Edition), Cold Spring Harbor Laboratory Press, 2012. Alternatively, the antigen may be purified from natural sources, e.g., purified from cells using protein separation techniques that are well known in the art. Such purification techniques may include, molecular sieve chromatography, ion exchange chromatography, etc.

In some embodiments, the sample is diluted or undiluted. The sample can be from about 1 to about 500 microliters, about 1 to about 450 microliters, about 1 to about 400 microliters, about 1 to about 350 microliters, about 1 to about 300 microliters, about 1 to about 250 microliters, about 1 to about 200 microliters, about 1 to about 150 microliters, about 1 to about 100 microliters, about 1 to about 90 microliters, about 1 to about 80 microliters, about 1 to about 75 microliters, about 1 to about 70 microliters, about 1 to about 60 microliters, about 1 to about 50 microliters, about 1 to about 40 microliters, about 1 to about 30 microliters, about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, or about 1 microliter to about 15 microliters.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety.

In some embodiments, measuring the level of brain biomarker autoantibodies includes contacting the sample with a first specific binding member and second specific binding member. In some embodiments the first specific binding member is a capture antibody and the second specific binding member is a detection antibody. In some embodiments, measuring the level of brain biomarker includes contacting the sample, either simultaneously or sequentially, in any order: (1) a capture antibody (e.g., a brain biomarker-capture antibody), which binds to a brain biomarker autoantibody to form a capture antibody-brain biomarker autoantibody complex (e.g., a brain biomarker-capture antibody-brain biomarker autoantibody complex), and (2) a detection antibody (e.g., brain biomarker-detection antibody), which includes a detectable label and binds to a different location on the brain biomarker autoantibody that is not bound by the capture antibody, to form a brain biomarker autoantibody-detection antibody complex (e.g., a brain biomarker autoantibody-brain biomarker-detection antibody complex), such that a capture antibody-brain biomarker autoantibody-detection antibody complex (e.g., a brain biomarker-capture antibody-brain biomarker autoantibody-brain biomarker-detection antibody complex) is formed, and measuring the amount or concentration of brain biomarker in the sample based on the signal generated by the detectable label in the capture antibody-brain biomarker autoantibody-detection antibody complex.

In some embodiments, the first specific binding member is immobilized on a solid support. In some embodiments, the second specific binding member is immobilized on a solid support. In some embodiments, the first specific binding member is an anti-brain biomarker antibody as described previously herein.

In some embodiments, the sample is diluted or undiluted. The sample can be from about 1 to about 500 microliters, about 1 to about 450 microliters, about 1 to about 400 microliters, about 1 to about 350 microliters, about 1 to about 300 microliters, about 1 to about 250 microliters, about 1 to about 200 microliters, about 1 to about 150 microliters, about 1 to about 100 microliters, about 1 to about 90 microliters, about 1 to about 80 microliters, about 1 to about 75 microliters, about 1 to about 70 microliters, about 1 to about 60 microliters, about 1 to about 50 microliters, about 1 to about 40 microliters, about 1 to about 30 microliters, about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, or about 1 microliter to about 15 microliters.

Some instruments (such as, for example the Abbott Laboratories instrument ARCHITECT®, and other core laboratory instruments) other than a point-of-care device may be capable of measuring levels of brain biomarker in a sample higher or greater than 25,000 pg/mL.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety.

### 8. Variations on Methods

The disclosed methods of determining the presence or amount of analyte of interest (e.g., one or more biomarkers) present in a sample may be as described herein. The methods may also be adapted in view of other methods for analyzing analytes. Examples of well-known variations include, but are not limited to, immunoassay, such as sandwich immunoassay (e.g., monoclonal-monoclonal sandwich immunoassays, monoclonal-polyclonal sandwich immunoassays, including enzyme detection (enzyme immunoassay (EIA) or enzyme-linked immunosorbent assay (ELISA), competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, etc.

a. Immunoassay

The analyte of interest, and/or peptides of fragments thereof (e.g., one or more biomarkers, and/or peptides or fragments thereof), may be analyzed using one or more biomarker antibodies in an immunoassay. The presence or amount of analyte (e.g., one or more biomarkers) can be determined using antibodies and detecting specific binding to the analyte (e.g., one or more biomarkers). For example, the antibody, or antibody fragment thereof, may specifically bind to the analyte (e.g., one or more biomarkers). If desired, one or more of the antibodies can be used in combination with one or more commercially available monoclonal/polyclonal antibodies. Such antibodies are available from companies such as R&D Systems, Inc. (Minneapolis, Minn.) and Enzo Life Sciences International, Inc. (Plymouth Meeting, Pa.).

The presence or amount of analyte (e.g., one or more biomarkers) present in a body sample may be readily determined using an immunoassay, such as sandwich immunoassay (e.g., monoclonal-monoclonal sandwich immunoassays, monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)). An example of a point-of-care device that can be used is i-STAT® (Abbott, Laboratories, Abbott Park, Ill.). Other methods that can be used include a chemiluminescent microparticle immunoassay, in particular one employing the ARCHITECT® automated analyzer (Abbott Laboratories, Abbott Park, Ill.), as an example. Other methods include, for example, mass spectrometry, and immunohistochemistry (e.g., with sections from tissue biopsies), using anti-analyte (e.g., an anti-biomarker) antibodies (monoclonal, polyclonal, chimeric, humanized, human, etc.) or antibody fragments thereof against analyte (e.g., one or more biomarkers). Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of the antibody to the analyte (e.g., one or more biomarkers) can be detected via direct labels, such as fluorescent or luminescent tags, metals and radionuclides attached to the antibody or via indirect labels, such as alkaline phosphatase or horseradish peroxidase.

The use of immobilized antibodies or antibody fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

A homogeneous format may be used. For example, after the test sample is obtained from a subject, a mixture is prepared. The mixture contains the test sample being assessed for analyte (e.g., one or more biomarkers), a first specific binding partner, and a second specific binding partner. The order in which the test sample, the first specific binding partner, and the second specific binding partner are added to form the mixture is not critical. The test sample is simultaneously contacted with the first specific binding partner and the second specific binding partner. In some embodiments, the first specific binding partner and any one or more biomarkers contained in the test sample may form a first specific binding partner-analyte (e.g., one or more biomarkers)-antigen complex and the second specific binding partner may form a first specific binding partner-analyte of interest (e.g., one or more biomarker)-second specific binding partner complex. In some embodiments, the second specific binding partner and any contained in the test sample may form a second specific binding partner-analyte (e.g., biomarker)-antigen complex and the first specific binding partner may form a first specific binding partner-analyte of interest (e.g., one or more biomarker)-second specific binding partner complex. For example, the first specific binding partner may be an anti-analyte antibody (e.g., anti-NSE antibody that binds to an epitope having an amino acid sequence comprising at least three contiguous (3) amino acids of SEQ ID NO: 6 or anti-NF-L antibody that binds to an epitope having an amino acid sequence comprising at least three contiguous (3) amino acids of SEQ ID NO: 7. The second specific binding partner may be an anti-analyte antibody (e.g., anti-NSE antibody that binds to an epitope having an amino acid sequence comprising at least three contiguous (3) amino acids of SEQ ID NO: 6 or anti-NF-L antibody that binds to an epitope having an amino acid sequence comprising at least three contiguous (3) amino acids of SEQ ID NO: 7). Moreover, the second specific binding partner is labeled with or contains a detectable label as described above.

A heterogeneous format may be used. For example, after the test sample is obtained from a subject, a first mixture is prepared. The mixture contains the test sample being assessed for analyte (e.g., one or more biomarkers) and a first specific binding partner, wherein the first specific binding partner and any One or more biomarkers contained in the test sample form a first specific binding partner-analyte (e.g., one or more biomarker)-antigen complex. For example, the first specific binding partner may be an anti-analyte antibody (e.g., anti-NSE antibody that binds to an epitope having an amino acid sequence comprising at least three contiguous (3) amino acids of SEQ ID NO: 6 or anti-NF-L antibody that binds to an epitope having an amino acid sequence comprising at least three contiguous (3) amino acids of SEQ ID NO: 7). The order in which the test sample and the first specific binding partner are added to form the mixture is not critical.

The first specific binding partner may be immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc, and a chip. In those embodiments where the solid phase is a bead, the bead may be a magnetic bead or a magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO.Fe₂O₃). Beads can have a solid core portion that is magnetic and is surrounded by one or more non-magnetic layers. Alternately, the magnetic portion can be a layer around a non-magnetic core. The solid support on which the first specific binding member is immobilized may be stored in dry form or in a liquid. The magnetic beads may be subjected to a magnetic field prior to or after contacting with the sample with a magnetic bead on which the first specific binding member is immobilized.

After the mixture containing the first specific binding partner-analyte (e.g., one or more biomarker) antigen complex is formed, any unbound analyte (e.g., one or more biomarkers) is removed from the complex using any technique known in the art. For example, the unbound analyte (e.g., one or more biomarkers) can be removed by washing. Desirably, however, the first specific binding partner is present in excess of any analyte (e.g., one or more biomarkers) present in the test sample, such that all analyte (e.g., one or more biomarkers) that is present in the test sample is bound by the first specific binding partner.

After any unbound analyte (e.g., one or more biomarkers) is removed, a second specific binding partner is added to the mixture to form a first specific binding partner-analyte of interest (e.g., one or more biomarkers)-second specific binding partner complex. For example, the second specific binding partner may be an anti-analyte antibody (e.g., anti-NSE antibody that binds to an epitope having an amino acid sequence comprising at least three contiguous (3) amino acids of SEQ ID NO: 6 or anti-NF-L antibody that binds to an epitope having an amino acid sequence comprising at least three contiguous (3) amino acids of SEQ ID NO: 7). Moreover, the second specific binding partner is labeled with or contains a detectable label as described above.

The use of immobilized antibodies or antibody fragments thereof may be incorporated into the immunoassay. The antibodies may be immobilized onto a variety of supports, such as magnetic or chromatographic matrix particles (such as a magnetic bead), latex particles or modified surface latex particles, polymer or polymer film, plastic or plastic film, planar substrate, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

(1) Sandwich Immunoassay

A sandwich immunoassay measures the amount of antigen between two layers of antibodies (e.g., at least one capture antibody) and a detection antibody (e.g., at least one detection antibody). The capture antibody and the detection antibody bind to different epitopes on the antigen, e.g., analyte of interest such as one or more biomarkers. Desirably, binding of the capture antibody to an epitope does not interfere with binding of the detection antibody to an epitope. Either monoclonal or polyclonal antibodies may be used as the capture and detection antibodies in the sandwich immunoassay.

Generally, at least two antibodies are employed to separate and quantify analyte (e.g., one or more biomarkers) in a test sample. More specifically, the at least two antibodies bind to certain epitopes of analyte (e.g., one or more biomarkers) forming an immune complex which is referred to as a “sandwich”. One or more antibodies can be used to capture the analyte (e.g., one or more biomarkers) in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, the binding of an antibody to its epitope desirably is not diminished by the binding of any other antibody in the assay to its respective epitope. Antibodies are selected so that the one or more first antibodies brought into contact with a test sample suspected of containing analyte (e.g., one or more biomarkers) do not bind to all or part of an epitope recognized by the second or subsequent antibodies, thereby interfering with the ability of the one or more second detection antibodies to bind to the analyte (e.g., one or more biomarkers).

The antibodies may be used as a first antibody in said immunoassay. The antibody immunospecifically binds to epitopes on analyte (e.g., one or more biomarkers). In addition to the antibodies of the present invention, said immunoassay may comprise a second antibody that immunospecifically binds to epitopes that are not recognized or bound by the first antibody.

A test sample suspected of containing analyte (e.g., one or more biomarkers) can be contacted with at least one first capture antibody (or antibodies) and at least one second detection antibodies either simultaneously or sequentially. In the sandwich assay format, a test sample suspected of containing analyte (e.g., one or more biomarkers) is first brought into contact with the at least one first capture antibody that specifically binds to a particular epitope under conditions which allow the formation of a first antibody-analyte (e.g., one or more biomarkers) antigen complex. If more than one capture antibody is used, a first multiple capture antibody-one or more biomarker antigen complex is formed. In a sandwich assay, the antibodies, preferably, the at least one capture antibody, are used in molar excess amounts of the maximum amount of analyte (e.g., one or more biomarkers) expected in the test sample. For example, from about 5 μg/mL to about 1 mg/mL of antibody per ml of microparticle coating buffer may be used.

i. Capture Antibody

Optionally, prior to contacting the test sample with the at least one first capture antibody, the at least one first capture antibody can be bound to a solid support which facilitates the separation the first antibody-analyte (e.g., one or more biomarkers) complex from the test sample. Any solid support known in the art can be used, including but not limited to, solid supports made out of polymeric materials in the forms of wells, tubes, or beads (such as a microparticle). The antibody (or antibodies) can be bound to the solid support by adsorption, by covalent bonding using a chemical coupling agent or by other means known in the art, provided that such binding does not interfere with the ability of the antibody to bind analyte (e.g., one or more biomarkers). Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide).

After the test sample suspected of containing analyte (e.g., one or more biomarkers) is incubated in order to allow for the formation of a first capture antibody (or multiple antibody)-analyte (e.g., one or more biomarkers) complex. The incubation can be carried out at a pH of from about 4.5 to about 10.0, at a temperature of from about 2° C. to about 45° C., and for a period from at least about one (1) minute to about eighteen (18) hours, from about 2-6 minutes, from about 7-12 minutes, from about 5-15 minutes, or from about 3-4 minutes.

ii. Detection Antibody

After formation of the first/multiple capture antibody-analyte (e.g., one or more biomarkers) complex, the complex is then contacted with at least one second detection antibody (under conditions that allow for the formation of a first/multiple antibody-analyte (e.g., one or more biomarkers) antigen-second antibody complex). In some embodiments, the test sample is contacted with the detection antibody simultaneously with the capture antibody. If the first antibody-analyte (e.g., one or more biomarkers) complex is contacted with more than one detection antibody, then a first/multiple capture antibody-analyte (e.g., one or more biomarkers)-multiple antibody detection complex is formed. As with first antibody, when the at least second (and subsequent) antibody is brought into contact with the first antibody-analyte (e.g., one or more biomarkers) complex, a period of incubation under conditions similar to those described above is required for the formation of the first/multiple antibody-analyte (e.g., one or more biomarkers)-second/multiple antibody complex. Preferably, at least one second antibody contains a detectable label. The detectable label can be bound to the at least one second antibody prior to, simultaneously with or after the formation of the first/multiple antibody-analyte (e.g., one or more biomarkers)-second/multiple antibody complex. Any detectable label known in the art can be used.

Chemiluminescent assays can be performed in accordance with the methods described in Adamczyk et al., Anal. Chim. Acta 579(1): 61-67 (2006). While any suitable assay format can be used, a microplate chemiluminometer (Mithras LB-940, Berthold Technologies U.S.A., LLC, Oak Ridge, Tenn.) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed.

The order in which the test sample and the specific binding partner(s) are added to form the mixture for chemiluminescent assay is not critical. If the first specific binding partner is detectably labeled with an acridinium compound, detectably labeled first specific binding partner-antigen (e.g., one or more biomarker) complexes form. Alternatively, if a second specific binding partner is used and the second specific binding partner is detectably labeled with an acridinium compound, detectably labeled first specific binding partner-analyte (e.g., one or more biomarker)-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of analyte (e.g., one or more biomarkers) is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample. Other labels other than chemiluminescent labels can be employed. For instance, enzymatic labels (including but not limited to alkaline phosphatase) can be employed.

The chemiluminescent signal, or other signal, that is generated can be detected using routine techniques known to those skilled in the art. Based on the intensity of the signal generated, the amount of analyte of interest (e.g., one or more biomarkers) in the sample can be quantified. Specifically, the amount of analyte (e.g., one or more biomarkers) in the sample is proportional to the intensity of the signal generated. The amount of analyte (e.g., one or more biomarkers) present can be quantified by comparing the amount of light generated to a standard curve for analyte (e.g., one or more biomarkers) or by comparison to a reference standard. The standard curve can be generated using serial dilutions or solutions of known concentrations of analyte (e.g., one or more biomarkers) by mass spectroscopy, gravimetric methods, and other techniques known in the art.

(1) Forward Competitive Inhibition Assay

In a forward competitive format, an aliquot of labeled analyte of interest (e.g., analyte (e.g., one or more biomarkers) having a fluorescent label, a tag attached with a cleavable linker, etc.) of a known concentration is used to compete with analyte of interest (e.g., one or more biomarkers) in a test sample for binding to analyte of interest antibody (e.g., one or more biomarker antibodies).

In a forward competition assay, an immobilized specific binding partner (such as an antibody) can either be sequentially or simultaneously contacted with the test sample and a labeled analyte of interest, analyte of interest fragment or analyte of interest variant thereof. The analyte of interest peptide, analyte of interest fragment or analyte of interest variant can be labeled with any detectable label, including a detectable label comprised of tag attached with a cleavable linker. In this assay, the antibody can be immobilized on to a solid support. Alternatively, the antibody can be coupled to an antibody, such as an antispecies antibody, that has been immobilized on a solid support, such as a microparticle or planar substrate.

The labeled analyte of interest, the test sample and the antibody are incubated under conditions similar to those described above in connection with the sandwich assay format. Two different species of antibody-analyte of interest complexes may then be generated. Specifically, one of the antibody-analyte of interest complexes generated contains a detectable label (e.g., a fluorescent label, etc.) while the other antibody-analyte of interest complex does not contain a detectable label. The antibody-analyte of interest complex can be, but does not have to be, separated from the remainder of the test sample prior to quantification of the detectable label. Regardless of whether the antibody-analyte of interest complex is separated from the remainder of the test sample, the amount of detectable label in the antibody-analyte of interest complex is then quantified. The concentration of analyte of interest (such as membrane-associated analyte of interest, soluble analyte of interest, fragments of soluble analyte of interest, variants of analyte of interest (membrane-associated or soluble analyte of interest) or any combinations thereof) in the test sample can then be determined, e.g., as described above.

(2) Reverse Competitive Inhibition Assay

In a reverse competition assay, an immobilized analyte of interest (e.g., one or more biomarkers) can either be sequentially or simultaneously contacted with a test sample and at least one labeled antibody.

The analyte of interest can be bound to a solid support, such as the solid supports discussed above in connection with the sandwich assay format.

The immobilized analyte of interest, test sample and at least one labeled antibody are incubated under conditions similar to those described above in connection with the sandwich assay format. Two different species analyte of interest-antibody complexes are then generated. Specifically, one of the analyte of interest-antibody complexes generated is immobilized and contains a detectable label (e.g., a fluorescent label, etc.) while the other analyte of interest-antibody complex is not immobilized and contains a detectable label. The non-immobilized analyte of interest-antibody complex and the remainder of the test sample are removed from the presence of the immobilized analyte of interest-antibody complex through techniques known in the art, such as washing. Once the non-immobilized analyte of interest antibody complex is removed, the amount of detectable label in the immobilized analyte of interest-antibody complex is then quantified following cleavage of the tag. The concentration of analyte of interest in the test sample can then be determined by comparing the quantity of detectable label as described above.

(3) One-Step Immunoassay or “Capture on the Fly” Assay

In a capture on the fly immunoassay, a solid substrate is pre-coated with an immobilization agent. The capture agent, the analyte (e.g., one or more biomarkers) and the detection agent are added to the solid substrate together, followed by a wash step prior to detection. The capture agent can bind the analyte (e.g., one or more biomarkers) and comprises a ligand for an immobilization agent. The capture agent and the detection agents may be antibodies or any other moiety capable of capture or detection as described herein or known in the art. The ligand may comprise a peptide tag and an immobilization agent may comprise an anti-peptide tag antibody. Alternately, the ligand and the immobilization agent may be any pair of agents capable of binding together so as to be employed for a capture on the fly assay (e.g., specific binding pair, and others such as are known in the art). More than one analyte may be measured. In some embodiments, the solid substrate may be coated with an antigen and the analyte to be analyzed is an antibody.

In certain other embodiments, in a one-step immunoassay or “capture on the fly”, a solid support (such as a microparticle) pre-coated with an immobilization agent (such as biotin, streptavidin, etc.) and at least a first specific binding member and a second specific binding member (which function as capture and detection reagents, respectively) are used. The first specific binding member comprises a ligand for the immobilization agent (for example, if the immobilization agent on the solid support is streptavidin, the ligand on the first specific binding member may be biotin) and also binds to the analyte of interest (e.g., one or more biomarkers). The second specific binding member comprises a detectable label and binds to an analyte of interest (e.g., one or more biomarkers). The solid support and the first and second specific binding members may be added to a test sample (either sequentially or simultaneously). The ligand on the first specific binding member binds to the immobilization agent on the solid support to form a solid support/first specific binding member complex. Any analyte of interest present in the sample binds to the solid support/first specific binding member complex to form a solid support/first specific binding member/analyte complex. The second specific binding member binds to the solid support/first specific binding member/analyte complex and the detectable label is detected. An optional wash step may be employed before the detection. In certain embodiments, in a one-step assay more than one analyte may be measured. In certain other embodiments, more than two specific binding members can be employed. In certain other embodiments, multiple detectable labels can be added. In certain other embodiments, multiple analytes of interest can be detected, or their amounts, levels or concentrations, measured, determined or assessed.

The use of a capture on the fly assay can be done in a variety of formats as described herein, and known in the art. For example, the format can be a sandwich assay such as described above, but alternately can be a competition assay, can employ a single specific binding member, or use other variations such as are known.

### 9. Other Factors

The methods of diagnosing, prognosticating, and/or assessing, as described above, can further include using other factors for the diagnosis, prognostication, and assessment. In some embodiments, traumatic brain injury may be diagnosed using the Glasgow Coma Scale or the Extended Glasgow Outcome Scale (GOSE). Other tests, scales or indices can also be used either alone or in combination with the Glasgow Coma Scale. An example is the Ranchos Los Amigos Scale. The Ranchos Los Amigos Scale measures the levels of awareness, cognition, behavior and interaction with the environment. The Ranchos Los Amigos Scale includes: Level I: No Response; Level II: Generalized Response; Level III: Localized Response; Level IV: Confused-agitated; Level V: Confused-inappropriate; Level VI: Confused-appropriate; Level VII: Automatic-appropriate; and Level VIII: Purposeful-appropriate.

### 10. Samples

In some embodiments, the sample is obtained after the human subject sustained an injury to the head caused by physical shaking, blunt impact by an external mechanical or other force that results in a closed or open head trauma, one or more falls, explosions or blasts or other types of blunt force trauma. In some embodiments, the sample is obtained after the human subject has ingested or been exposed to a chemical, toxin or combination of a chemical and toxin. Examples of such chemicals and/or toxins include, fires, molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. In some embodiments, the sample is obtained from a human subject that suffers from an autoimmune disease, a metabolic disorder, a brain tumor, hypoxia, one or more viruses, meningitis, hydrocephalus or combinations thereof.

In yet another embodiment, the methods described herein use samples that also can be used to determine whether or not a subject has or is at risk of developing mild traumatic brain injury by determining the levels of one or more biomarkers in a subject using the anti-biomarkers antibodies described below, or antibody fragments thereof. Thus, in particular embodiments, the disclosure also provides a method for determining whether a subject having, or at risk for, traumatic brain injuries, discussed herein and known in the art, is a candidate for therapy or treatment. Generally, the subject is at least one who: (i) has experienced an injury to the head; (ii) ingested and/or been exposed to one or more chemicals and/or toxins; (iii) suffers from an autoimmune disease, a metabolic disorder, a brain tumor, hypoxia, one or more viruses, meningitis, hydrocephalus or suffers from any combinations thereof; or (iv) any combinations of (i)-(iii); or, who has actually been diagnosed as having, or being at risk for TBI (such as, for example, subjects suffering from an autoimmune disease, a metabolic disorder, a brain tumor, hypoxia, one or more viruses, meningitis, hydrocephalus or combinations thereof), and/or who demonstrates an unfavorable (e.g., clinically undesirable) concentration or amount of one or more biomarkers or fragments thereof, as described herein.

a. Test or Biological Sample

As used herein, “sample”, “test sample”, “biological sample” refer to fluid sample containing or suspected of containing one or more biomarkers. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing one or more biomarkers may be assayed directly. In a particular example, the source of one or more biomarkers is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 150 μL about 200 μL, about 300 μL, about 400 μL, about 500 μL about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, between about 0.01 μL and about 150 μL, between about 0.01 μL and about 200 μL, between about 0.01 μL and about 300 μL, between about 0.01 μL and about 400 μL, between about 0.01 μL and about 500 μL, or between about 0.1 μL and about 10 μL.

In some cases, the fluid sample may be diluted prior to use in an assay. For example, in embodiments where the source of one or more biomarkers is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments where the source of one or more biomarkers is a human body fluid (e.g., blood, serum), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

b. Controls

It may be desirable to include a control sample. The control sample may be analyzed concurrently with the sample from the subject as described above. The results obtained from the subject sample can be compared to the results obtained from the control sample. Standard curves may be provided, with which assay results for the sample may be compared. Such standard curves present levels of marker as a function of assay units, e.g., fluorescent signal intensity, if a fluorescent label is used. Using samples taken from multiple donors, standard curves can be provided for reference levels of one or more biomarkers in normal healthy tissue, as well as for “at-risk” levels of the one or more biomarkers in tissue taken from donors, who may have one or more of the characteristics set forth above.

Thus, in view of the above, a method for determining the presence, amount, or concentration of one or more biomarkers in a test sample is provided. The method comprises assaying the test sample for one or more biomarkers by an immunoassay, for example, employing at least one capture antibody that binds to an epitope on a biomarker and at least one detection antibody that binds to an epitope on a biomarker which is different from the epitope for the capture antibody and optionally includes a detectable label, and comprising comparing a signal generated by the detectable label as a direct or indirect indication of the presence, amount or concentration of a biomarker in the test sample to a signal generated as a direct or indirect indication of the presence, amount or concentration of a biomarker in a calibrator. The calibrator is optionally, and is preferably, part of a series of calibrators in which each of the calibrators differs from the other calibrators in the series by the concentration of a biomarker.

### 11. Kit

Provided herein is a kit, which may be used for assaying or assessing a test sample for one or more biomarkers or fragments thereof. The kit comprises at least one component for assaying the test sample for one or more biomarkers. For example, the kit can comprise instructions for assaying the test sample for one or more biomarkers by immunoassay, e.g., chemiluminescent microparticle immunoassay. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The at least one component may include at least one composition comprising one or more isolated antibodies or antibody fragments thereof that specifically bind to, for example, NSE and/or NF-L. The antibody may be one or more biomarker capture antibodies and/or one or more biomarker detection antibodies.

Alternatively or additionally, the kit can comprise a calibrator or control, e.g., purified, and optionally lyophilized, biomarker(s), and/or at least one container (e.g., tube, microtiter plates or strips, which can be already coated with an anti-biomarker monoclonal antibody(ies)) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, e.g., reagents, standards, buffers, diluents, etc., which are necessary to perform the assay. The instructions also can include instructions for generating a standard curve.

The kit may further comprise reference standards for quantifying the one or more biomarkers. The reference standards may be employed to establish standard curves for interpolation and/or extrapolation of one or more biomarker concentrations. The reference standards may include a high one or more biomarker concentration level, for example, about 100000 pg/mL, about 125000 pg/mL, about 150000 pg/mL, about 175000 pg/mL, about 200000 pg/mL, about 225000 pg/mL, about 250000 pg/mL, about 275000 pg/mL, or about 300000 pg/mL; a medium One or more biomarker concentration level, for example, about 25000 pg/mL, about 40000 pg/mL, about 45000 pg/mL, about 50000 pg/mL, about 55000 pg/mL, about 60000 pg/mL, about 75000 pg/mL or about 100000 pg/mL; and/or a low One or more biomarker concentration level, for example, about 1 pg/mL, about 5 pg/mL, about 10 pg/mL, about 12.5 pg/mL, about 15 pg/mL, about 20 pg/mL, about 25 pg/mL, about 30 pg/mL, about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, about 80 pg/mL, about 85 pg/mL, about 90 pg/mL, about 95 pg/mL, or about 100 pg/mL.

Any antibodies, which are provided in the kit, such as recombinant antibodies specific for one or more biomarkers, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes (e.g., one or more biomarkers) or reagents for detecting the analyte (e.g., one or more biomarkers). The antibodies, calibrators, and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays,

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc, or chip.

If desired, the kit can further comprise one or more components, alone or in further combination with instructions, for assaying the test sample for another analyte, which can be a biomarker, such as a biomarker of traumatic brain injury or disorder.

a. Adaptation of Kit and Method

The kit (or components thereof), as well as the method for assessing or determining the concentration of one or more biomarkers in a test sample by an immunoassay as described herein, can be adapted for use in a variety of automated and semi-automated systems (including those wherein the solid phase comprises a microparticle), as described, e.g., U.S. Pat. No. 5,063,081, U.S. Patent Application Publication Nos. 2003/0170881, 2004/0018577, 2005/0054078, and 2006/0160164 and as commercially marketed e.g., by Abbott Laboratories (Abbott Park, Ill.) as Abbott Point of Care (i-STAT® or i-STAT Alinity, Abbott Laboratories) as well as those described in U.S. Pat. Nos. 5,089,424 and 5,006,309, and as commercially marketed, e.g., by Abbott Laboratories (Abbott Park, Ill.) as ARCHITECT® or the series of Abbott Alinity® devices.

Some of the differences between an automated or semi-automated system as compared to a non-automated system (e.g., ELISA) include the substrate to which the first specific binding partner (e.g., analyte antibody or capture antibody) is attached (which can affect sandwich formation and analyte reactivity), and the length and timing of the capture, detection, and/or any optional wash steps. Whereas a non-automated format such as an ELISA may require a relatively longer incubation time with sample and capture reagent (e.g., about 2 hours), an automated or semi-automated format (e.g., ARCHITECT® and any successor platform, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits, and kit components can be employed in other formats, for example, on electrochemical or other hand-held or point-of-care assay systems. As mentioned previously, the present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Patent App. Publication Nos. 2003/0170881, 2004/0018577, 2005/0054078, and 2006/0160164, which are incorporated in their entireties by reference for their teachings regarding same.

In particular, with regard to the adaptation of an assay to the i-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an i-STAT® cartridge with a fluidics format suitable for immunoassay. On a portion of the silicon chip, there is a specific binding partner for one biomarker, such as one or more biomarker antibodies (one or more monoclonal/polyclonal antibody or a fragment thereof, a variant thereof, or a fragment of a variant thereof that can bind a biomarker) or one or more anti-biomarker-DVD-Igs (or a fragment thereof, a variant thereof, or a fragment of a variant thereof that can bind a biomarker), either of which can be detectably labeled. Within the fluid pouch of the cartridge is an aqueous reagent that includes p-aminophenol phosphate.

In operation, a sample from a subject suspected of suffering from TBI is added to the holding chamber of the test cartridge, and the cartridge is inserted into the i-STAT® reader. A pump element within the cartridge pushes the sample into a conduit containing the chip. The sample is brought into contact with the sensors allowing the enzyme conjugate to dissolve into the sample. The sample is oscillated across the sensors to promote formation of the sandwich of approximately 2-12 minutes. In the penultimate step of the assay, the sample is pushed into a waste chamber and wash fluid, containing a substrate for the alkaline phosphatase enzyme, is used to wash excess enzyme conjugate and sample off the sensor chip. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of a biomarker in the sample by means of an embedded algorithm and factory-determined calibration curve.

The methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an i-STAT® cartridge format, using a nucleic acid sequence linked to the signal antibody as a signal amplifier.

While certain embodiments herein are advantageous when employed to assess disease, such as traumatic brain injury, the assays and kits also optionally can be employed to assess one or more biomarkers in other diseases, disorders, and conditions as appropriate.

The method of assay also can be used to identify a compound that ameliorates diseases, such as traumatic brain injury. For example, a cell that expresses one or more biomarkers can be contacted with a candidate compound. The level of expression of one or more biomarkers in the cell contacted with the compound can be compared to that in a control cell using the method of assay described herein.

The present invention has multiple aspects, illustrated by the following non-limiting examples.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present invention has multiple aspects, illustrated by the following non-limiting examples.

### Example 1: Blood Biomarker Changes in a Comparative Mild Traumatic Brain Injury Study

This study compares the biomarker profiles of two mild TBI (mTBI) cases with intracranial abnormalities with matching healthy controls to investigate diagnostic utilization of blood based TBI biomarkers for assessing (e.g., predicting the presence of) neuroimaging abnormalities in head trauma patients.

As part of a sponsored study of imaging and blood biomarkers of mTBI, patients were recruited from participating clinical sites alongside demographically matched controls. Magnetic resonance imaging (MRI) was conducted using 3T MRI scanners (GE Signa MR750; GE Healthcare, Waukesha, Wis.) with a protocol that included high-resolution anatomical sequences (MP-RAGE, T2 FLAIR, SWAN), multi-shell diffusion MRI, resting state fMRI and ASL perfusion. Anatomical images were read by site neuro-radiologists. Candidate TBI biomarkers GFAP and UCH-L1 were measured in plasma samples collected from patients and controls using iSTAT platform immunoassays (Abbott Laboratories, IL), described for example in WO 2018/067468 and WO 2018/067474, the contents of which are herein incorporated by reference.

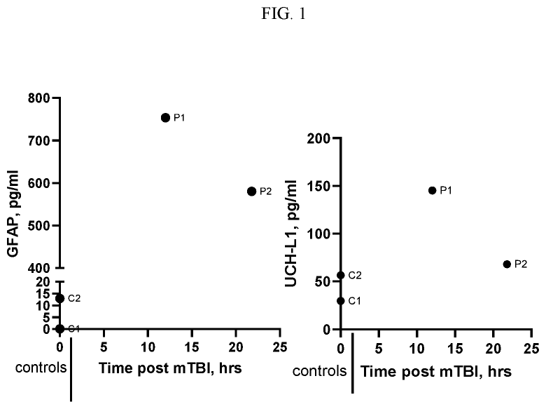

Among 135 patients in the recruited cohort, 24 had an abnormal MRI but only 3 of these abnormal reads were associated with brain trauma. Two out of these 3 patients had a blood sample collected within the first 24 hours post injury. MRI findings from patient 1 (P1), a 23-year-old female, indicated numerous periventricular white matter hyperintensities. Patient 2 (P2), a 21-year-old male, was identified to have small hemorrhagic contusions with adjacent white matter hemorrhagic foci. In the blood sample from P1, collected at 12 hours post-injury, level of GFAP was 753.6 pg/ml, level of UCH-L1 was 145.2 pg/ml. In the blood sample collected from P2 at 21.2 hours post-injury level of GFAP was 580.3 pg/ml, while UCH-L1 was at 68 pg/ml. In control subjects, a 22 year old male and a 22 year old female without any MRI abnormalities, GFAP levels were 0.1 and 12.9 pg/ml; and UCH-L1 was at 29.7 and 56.6 pg/ml, respectively (See, for example FIG. 1).

A comparison of biomarker profiles of two mTBI cases shows that levels of GFAP and UCH-L1 are higher at 12 hours compared to 21 hours post injury, and confirmed that GFAP shows potential diagnostic value at both timepoints. These studies confirm that blood biomarkers can predict the presence of neuroimaging abnormalities in head trauma patients. This study highlights the importance of biomarker testing timing for selection and interpretation of data.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A method of evaluating a human subject to determine whether to perform an advanced magnetic resonance imaging (MRI) procedure for a head injury, the method comprising:
a) performing an assay on one or more samples obtained from the subject after an actual or suspected injury to the head to measure or detect a level of one or more biomarkers of traumatic brain injury (TBI); and
b) determining whether to perform an advanced MRI procedure on the subject when the level of the one or more biomarkers in the one or more samples are unfavorable when compared to one or more reference levels of the one or more biomarkers and not performing an advanced MRI procedure on the subject when the level of the one or more biomarkers in the one or more samples are favorable when compared to one or more reference levels of the one or more biomarkers.

Clause 2. The method of clause 1, wherein the subject has received a conventional MRI procedure or an advanced MRI procedure before or after the assay is performed.

Clause 3. The method of clause 2, wherein the subject is suspected of having or is confirmed as having a head injury based on the conventional MRI procedure or advanced MRI procedure.

Clause 4. A method of aiding in the diagnosis and evaluation of a human subject that has sustained or may have sustained an injury to the head, the method comprising the steps of:
a) performing at least one assay on one or more samples taken from the subject to measure or detect a level of one or more biomarkers of traumatic brain injury (TBI);
b) determining the amount of said one or more biomarkers in the one or more samples;
c) performing at least one advanced magnetic resonance imaging (MRI) procedure on the subject;
d) obtaining data from the procedure using at least one computer-implemented method;
e) generating a TBI diagnosis for the subject using the amount of the one or more biomarkers determined in step b) and the data obtained in step d); and
f) determining whether the subject has sustained or not sustained a head injury based on the TBI diagnosis.

Clause 5. The method of clause 4, wherein if said subject is determined to have sustained a head injury, further assessing: (a) the pathophysiology of the head injury; (b) the endotype of the head injury; (c) whether the subject has sustained a mild, moderate, severe, or moderate to severe TBI; or (d) any combinations of (a)-(c).

Clause 6. The method clause 5, wherein if the subject is determined to have sustained a head injury, further assessing: (a) the pathophysiology of the head injury; (b) the endotype of the head injury; (c) whether the subject has sustained a mild, moderate, severe, or moderate to severe TBI; (d) the pathophysiology of the head injury and the endotype of the head injury; (e) the pathophysiology of the head injury and whether the subject has sustained a mild, moderate, severe, or moderate to severe TBI; (f) the endotype of the head injury and whether the subject has sustained a mild, moderate, severe, or moderate to severe TBI; or (g) the pathophysiology of the head injury, the endotype of the head injury and whether the subject has sustained a mild, moderate, severe, or moderate to severe TBI.

Clause 7. A method of aiding in the diagnosis and evaluation of a human subject that has sustained or may have sustained an injury to the head, the method comprising the steps of:
a) performing at least one assay on one or more samples taken from the subject to measure or detect a level of one or more biomarkers of traumatic brain injury (TBI);
b) determining the amount of said one or more biomarkers in the one or more samples;
c) performing at least one advanced magnetic resonance imaging (MRI) procedure on the subject;
d) obtaining data from the procedure using at least one computer-implemented method;
e) generating a TBI diagnosis for the subject using the amount of the one or more biomarkers determined in step b) and the data obtained in step d); and
f) determining whether the subject has or has not sustained a head injury based on the TBI diagnosis, wherein if the subject is determined to have sustained a head injury, determining (i) the pathophysiology of the head injury; (ii) the endotype of the head injury; or (iii) any combinations of (i)-(ii) based on the TBI diagnosis.

Clause 8. The method of clause 7, wherein if the subject is determined to have sustained a head injury, further assessing: (i) the pathophysiology of the head injury; (i) the endotype of the head injury; or (iii) the pathophysiology of the head injury and the endotype of the head injury.

Clause 9. The method of clauses 7-8, further comprising determining whether the subject has, or more likely than not has, sustained a mild, moderate, severe, or moderate to severe TBI.

Clause 10. The method of clause 9, wherein if the subject is determined to have sustained a head injury, further assessing: (i) the pathophysiology of the head injury; (ii) the endotype of the head injury; (iii) whether the subject has, or more likely than not has, sustained a mild, moderate, severe, or moderate to severe TBI; (iv) the pathophysiology of the head injury and the endotype of the head injury; (v) the pathophysiology of the head injury and whether the subject has, or more likely than not has, sustained a mild, moderate, severe, or moderate to severe TBI; (vi) the endotype of the head injury and whether the subject has, or more likely than not has, sustained a mild, moderate, severe, or moderate to severe TBI; or (vii) the pathophysiology of the head injury, the endotype of the head injury and whether the subject has, or more likely than not has, sustained a mild, moderate, severe, or moderate to severe TBI.

Clause 11. The method of clauses 4-10, wherein a first TBI diagnosis is generated for the subject based on the amount of the one or more biomarkers determined in step b).

Clause 12. The method of clauses 4-11, wherein a second TBI diagnosis is generated based on the data obtained in step d).

Clause 13. The method of clause 12, wherein it is determined whether the subject has not sustained a head injury or a head injury based on a combination of the first TBI diagnosis and the second TBI diagnosis.

Clause 14. A method for predicting or aiding in predicting the outcome of human subjects having a traumatic brain injury (TBI), the method comprising the steps of:
a) performing at least one assay on one or more samples taken from the subject to measure or detect a level of one or more biomarkers of TBI;
b) determining the amount of said one or more biomarkers in the one or more samples;
c) performing at least one advanced magnetic resonance imaging (MRI) procedure on the subject;
d) obtaining data from the procedure using at least one computer-implemented method;
e) generating a TBI outcome diagnosis for the subject using the amount of the one or more biomarkers determined in step b) and the data obtained in step d); and
f) predicting a favorable or unfavorable outcome in the subject based on the TBI outcome diagnosis.

Clause 15. The method of clause 14, wherein a first TBI outcome diagnosis is generated for the subject based on the amount of the one or more biomarkers determined in step b).

Clause 16. The method of clause 15, wherein a second TBI outcome diagnosis is generated based on the data obtained in step d).

Clause 17. The method of clause 16, wherein a favorable or unfavorable outcome is predicted for the subject based on a combination of the first TBI outcome diagnosis and the second TBI outcome diagnosis.

Clause 18. A method for predicting or aiding in predicting the outcome of human subjects having a traumatic brain injury (TBI), the method comprising the steps of:
a) performing at least one assay on at least one or more samples taken from the subject to measure or detect a level of one or more biomarkers of TBI;
b) performing an advanced magnetic resonance imaging (MRI) procedure on the subject when the level of the one or more biomarkers in the one or more samples is unfavorable when compared to one or more reference levels of the one or more biomarkers; and
c) predicting an outcome in the human subject based on the results of the advanced MRI procedure.

Clause 19. A method for determining a course of treatment or efficacy of a course of treatment for a human subject who has suffered a traumatic brain injury (TBI), the method comprising the steps of:
a) performing at least one assay on one or more samples taken from the subject to measure or detect a level of one or more biomarkers of TBI;
b) determining the amount of said one or more biomarkers in the one or more samples;
c) performing at least one advanced magnetic resonance imaging (MRI) procedure on the subject after diagnosis of a traumatic brain injury;
d) obtaining data from the at least one advanced MRI procedure performed in step c) using a computer-implemented method;
e) generating a TBI treatment protocol for the subject using the amount of the one or more biomarkers in the one or more samples obtained in step b) and the data obtained in step d) for assessing head injury; and
f) determine a course of treatment or the efficacy of a course of treatment in the subject based on the TBI treatment protocol.

Clause 20. The method of clause 19, wherein a first TBI treatment protocol is generated for the subject based on the amount of the one or more biomarkers determined in step b).

Clause 21. The method of clause 20, wherein a second TBI treatment protocol is generated based on the data obtained in step d).

Clause 22. The method of clause 21, wherein the course of treatment or the efficacy of the course of treatment is determined based on a combination of the first TBI treatment protocol and the second TBI treatment protocol.

Clause 23. The method of any one of clauses 1-22, wherein the one or more biomarkers are selected from the group consisting of: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b).

Clause 24. The method of clause 23, wherein the one or more biomarkers are: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

Clause 25. The method of any of clauses 1-24, wherein the advanced MRI procedure is gradient echo magnetic resonance imaging, echo planar magnetic resonance imaging, spin echo magnetization resonance imaging, diffusion MRI, resting-state functional MRI, magnetic resonance spectroscopy, perfusion magnetic resonance imaging, dynamic susceptibility weighted dynamic susceptibility MRI, dynamic contrast-enhanced MRI, quantitative structural MRI, magnetization transfer MRI or combinations thereof.

Clause 26. The method of any one of clauses 1-24, wherein the one or more samples is taken from the subject within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, with about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of the actual or suspected injury to the head.

Clause 27. The method of any of clauses 19-22, wherein the subject has been receiving treatment for the TBI.

Clause 28. The method of any of clauses 19-22, wherein the subject has not been receiving treatment for the TBI.

Clause 29. The method of any of clauses 14-28, wherein the subject has a mild TBI.

Clause 30. The method of any of clauses 14-28, wherein the subject has a moderate, severe or moderate to severe TBI.

Clause 31. The method of any of clauses 4-30, wherein the method further comprises monitoring the subject.

Clause 32. The method of any one of clauses 4-31, wherein the method further comprises administering a treatment for the TBI to the subject.

Clause 33. The method of clause 12 or 16, wherein the advanced MRI procedure identifies the location of the TBI in the brain of the subject.

Clause 34. The method of clause 33, wherein the subject is predicted of having a favorable outcome based on the location of the TBI.

Clause 35. The method of clause 33, wherein the subject is predicted of having an unfavorable outcome based on the location of the TBI.

Clause 36. The method of any of clauses 1-35, wherein the one or more biomarkers comprises two or more biomarkers.

Clause 37. The method of any of clauses 1-35, wherein the one or more biomarkers comprises four or more biomarkers.

Clause 38. The method of any one of clauses 1 to 37, wherein the sample is selected from the group consisting of a whole blood sample, a serum sample, a cerebrospinal fluid sample, and a plasma sample.

Clause 39. The method of any one of clauses 1 to 38, wherein the sample is obtained after the subject sustained an injury to the head caused by physical shaking, blunt impact by an external mechanical or other force that results in a closed or open head trauma, one or more falls, explosions or blasts or other types of blunt force trauma.

Clause 40. The method of any one of clauses 1 to 38, wherein the sample is obtained after the subject has ingested or been exposed to a chemical, toxin or combination of a chemical and toxin.

Clause 41. The method of clause 40, wherein the chemical or toxin is fire, mold, asbestos, a pesticide, an insecticide, an organic solvent, a paint, a glue, a gas, an organic metal, a drug of abuse or one or more combinations thereof.

Clause 42. The method of any one of clauses 1 to 38, wherein the sample is obtained from a subject that suffers from an autoimmune disease, a metabolic disorder, a brain tumor, hypoxia, a virus, meningitis, hydrocephalus or combinations thereof.

Clause 43. The method of any one of clauses 1 to 42, wherein said method can be carried out on any subject without regard to factors selected from the group consisting of the subject's clinical condition, the subject's laboratory values, the subject's classification as suffering from mild, moderate, severe, or moderate to severe traumatic brain injury, the subject's exhibition of low or high levels of one or more biomarkers, and the timing of any event wherein said subject may have sustained an injury to the head.

Clause 44. The method of any one of clauses 1 to 43, wherein the sample is a whole blood sample.

Clause 45. The method of any one of clauses 1 to 43, wherein the sample is a serum sample.

Clause 46. The method of any one of clauses 1 to 43, wherein the sample is a plasma sample.

Clause 47. The method of any one of clauses 1 to 46, wherein the assay is an immunoassay.

Clause 48. The method of any one of clauses 1 to 46, wherein the assay is a clinical chemistry assay.

Clause 49. The method of any one of clauses 1 to 46, wherein the assay is a single molecule detection assay.

Clause 50. The method of any one of clauses 4 to 10, wherein the pathophysiology of the head injury is determined by assessing whether the subject has or is suffering from at least one of cerebral hypofusion or hyperfusion, cerebral ischaemia, hyperaemia, impaired cerebrovascular CO₂-reactivity, a cerebral vasospasm, brain tissue hypoxia, oedema, oxidative stress, cell death or combinations thereof.

Clause 51. The method of clauses 4 to 10 and 50, wherein the endotype of the head injury is determined by determining (i) whether the injury is axonal, neuronal or astroglial; (ii) whether there is microvascular injury; (iii) whether there is proteinopathy; (iv) whether is there inflammation; or (v) any combinations thereof.

Clause 52. A method comprising:
a) performing an assay on one or more samples obtained from the subject after an actual or suspected injury to the head to measure or detect a level of one or more biomarkers of traumatic brain injury (TBI); and
b) performing an advanced magnetic resonance imaging (MRI) procedure on the subject when the level of the one or more biomarkers in the one or more samples are unfavorable when compared to one or more reference levels of the one or more biomarkers and not performing an advanced MRI procedure and treating the subject for a mild TBI when the level of the one or more biomarkers in the one or more samples are favorable when compared to one or more reference levels of the one or more biomarkers.

Clause 53. A method comprising the steps of:
a) performing at least one assay on one or more samples taken from the subject to measure or detect a level of one or more biomarkers of traumatic brain injury (TBI);
b) determining the amount of said one or more biomarkers in the one or more samples;
c) performing at least one advanced magnetic resonance imaging (MRI) procedure on the subject;
d) obtaining data from the procedure using at least one computer-implemented method;
e) generating a TBI diagnosis for the subject using the amount of the one or more biomarkers determined in step b) and the data obtained in step d);
f) determining that the subject has sustained a TBI based on the TBI diagnosis; and
g) treating the subject for the TBI.

Clause 54. The method of clause 53, wherein the subject is treated for a mild TBI.

Clause 55. The method of clause 53, wherein the subject is treated for a moderate, severe, or moderate to severe TBI.

Clause 56. A method comprising the steps of:
a) performing at least one assay on one or more samples taken from the subject to measure or detect a level of one or more biomarkers of traumatic brain injury (TBI);
b) determining the amount of said one or more biomarkers in the one or more samples;
c) performing at least one advanced magnetic resonance imaging (MRI) procedure on the subject;
d) obtaining data from the procedure using at least one computer-implemented method;
e) generating a TBI diagnosis for the subject using the amount of the one or more biomarkers determined in step b) and the data obtained in step d) and determining that the subject has sustained a TBI based on the TBI diagnosis;
f) determining (i) the pathophysiology of the TBI; (ii) the endotype of the TBI; or (iii) any combinations of (i)-(ii) endotype based on the TBI diagnosis; and
g) treating the subject for the TBI.

Clause 57. The method of clause 56, further comprising determining that the subject has sustained a mild, moderate, severe, or moderate to severe TBI.

Clause 58. The method of clauses 56 or 57, wherein the subject is treated for a mild TBI.

Clause 59. The method of clauses 56 or 57, wherein the subject is treated for a moderate, severe, or moderate to severe TBI.

What is claimed is:

1. A method of evaluating a human subject to determine whether to perform an advanced magnetic resonance imaging (MRI) procedure for a head injury, the method comprising:
a) performing an assay on one or more samples obtained from the subject after an actual or suspected injury to the head to measure or detect a level of one or more biomarkers of traumatic brain injury (TBI); and
b) determining whether to perform an advanced MRI procedure on the subject when the level of the one or more biomarkers in the one or more samples are unfavorable when compared to one or more reference levels of the one or more biomarkers and not performing an advanced MRI procedure on the subject when the level of the one or more biomarkers in the one or more samples are favorable when compared to one or more reference levels of the one or more biomarkers.

2. The method of claim 1, wherein the subject has received a conventional MRI procedure or an advanced MRI procedure before or after the assay is performed.

3. The method of claim 2, wherein the subject is suspected of having or is confirmed as having a head injury based on the conventional MRI procedure or advanced MRI procedure.

4. A method of aiding in the diagnosis and evaluation of a human subject that has sustained or may have sustained an injury to the head, the method comprising the steps of:
a) performing at least one assay on one or more samples taken from the subject to measure or detect a level of one or more biomarkers of traumatic brain injury (TBI);
b) determining the amount of said one or more biomarkers in the one or more samples;
c) performing at least one advanced magnetic resonance imaging (MRI) procedure on the subject;
d) obtaining data from the procedure using at least one computer-implemented method;
e) generating a TBI diagnosis for the subject using the amount of the one or more biomarkers determined in step b) and the data obtained in step d); and
f) determining whether the subject has sustained or not sustained a head injury based on the TBI diagnosis.

5. The method of claim 4, wherein if said subject is determined to have sustained a head injury, the method further comprises determining: (a) the pathophysiology of the head injury; (b) the endotype of the head injury; (c) whether the subject has, or more likely than not has, sustained a mild, moderate, severe, or moderate to severe TBI; or (d) any combinations of (a)-(c).

6. A method of aiding in the diagnosis and evaluation of a human subject that has sustained or may have sustained an injury to the head, the method comprising the steps of:
a) performing at least one assay on one or more samples taken from the subject to measure or detect a level of one or more biomarkers of traumatic brain injury (TBI);
b) determining the amount of said one or more biomarkers in the one or more samples;
c) performing at least one advanced magnetic resonance imaging (MRI) procedure on the subject;
d) obtaining data from the procedure using at least one computer-implemented method;
e) generating a TBI diagnosis for the subject using the amount of the one or more biomarkers determined in step b) and the data obtained in step d); and
f) determining whether the subject has sustained a head injury or has not sustained a head injury based on the TBI diagnosis, wherein if the subject is determined to have sustained a head injury, determining (i) the pathophysiology of the head injury; (ii) the endotype of the head injury; or (iii) any combinations of (i)-(ii) endotype based on the TBI diagnosis.

7. The method of claim 6, further comprising determining whether the subject has, or more likely than not has, sustained a mild, moderate, severe, or moderate to severe TBI.

8. The method of claims 4-7, wherein a first TBI diagnosis is generated for the subject based on the amount of the one or more biomarkers determined in step b).

9. The method of claims 4-8, wherein a second TBI diagnosis is generated based on the data obtained in step d).

10. The method of claim 9, wherein it is determined whether the subject has not sustained a head injury based on a combination of the first TBI diagnosis and the second TBI diagnosis.

11. A method for predicting or aiding in predicting the outcome of human subjects having a traumatic brain injury (TBI), the method comprising the steps of:
a) performing at least one assay on one or more samples taken from the subject to measure or detect a level of one or more biomarkers of TBI;
b) determining the amount of said one or more biomarkers in the one or more samples;
c) performing at least one advanced magnetic resonance imaging (MRI) procedure on the subject;
d) obtaining data from the procedure using at least one computer-implemented method;
e) generating a TBI outcome diagnosis for the subject using the amount of the one or more biomarkers determined in step b) and the data obtained in step d); and
f) predicting a favorable or unfavorable outcome in the subject based on the TBI outcome diagnosis.

12. The method of claim 11, wherein a first TBI outcome diagnosis is generated for the subject based on the amount of the one or more biomarkers determined in step b).

13. The method of claim 12, wherein a second TBI outcome diagnosis is generated based on the data obtained in step d).

14. The method of claim 13, wherein a favorable or unfavorable outcome is predicted for the subject based on a combination of the first TBI outcome diagnosis and the second TBI outcome diagnosis.

15. A method for predicting or aiding in predicting the outcome of human subjects having a traumatic brain injury (TBI), the method comprising the steps of:
a) performing at least one assay on at least one or more samples taken from the subject to measure or detect a level of one or more biomarkers of TBI;
b) performing an advanced magnetic resonance imaging (MRI) procedure on the subject when the level of the one or more biomarkers in the one or more samples is unfavorable when compared to one or more reference levels of the one or more biomarkers; and
c) predicting an outcome in the human subject based on the results of the advanced MRI procedure.

16. A method for determining a course of treatment or efficacy of a course of treatment for a human subject who has suffered a traumatic brain injury (TBI), the method comprising the steps of:
a) performing at least one assay on one or more samples taken from the subject to measure or detect a level of one or more biomarkers of TBI;
b) determining the amount of said one or more biomarkers in the one or more samples;
c) performing at least one advanced magnetic resonance imaging (MRI) procedure on the subject after diagnosis of a TBI;
d) obtaining data from the at least one advanced magnetic resonance imaging (MRI) procedure performed in step c) using a computer-implemented method;
e) generating a TBI treatment protocol for the subject using the amount of the one or more biomarkers in the one or more samples obtained in step b) and the data obtained in step d) for assessing head injury; and
f) determine a course of treatment or the efficacy of a course of treatment in the subject based on the TBI treatment protocol.

17. The method of claim 16, wherein a first TBI treatment protocol is generated for the subject based on the amount of the one or more biomarkers determined in step b).

18. The method of claim 17, wherein a second TBI treatment protocol is generated based on the data obtained in step d).

19. The method of claim 18, wherein the course of treatment or the efficacy of the course of treatment is determined based on a combination of the first TBI treatment protocol and the second TBI treatment protocol.

20. The method of any one of claims 1-19, wherein the one or more biomarkers are selected from the group consisting of: a) one or more biomarkers of neuronal or axonal injury, one or more biomarkers of astroglial injury, one or more biomarkers of neuroinflammation, one or more genetic biomarkers, and any combinations thereof; b) one or more autoantibodies to any of the biomarkers listed in a); and c) and combinations of a) and b).

21. The method of claim 20, wherein the one or more biomarkers are: a) APOA1, CRP, NSE, BDNF, tau, p-tau, NF-L, S100β, UCH-L1, GFAP and combinations thereof; b) one or more autoantibodies to any of the biomarkers listed in a); or c) combinations of a) and b).

22. The method of any of claims 1-21, wherein the advanced MRI procedure is gradient echo magnetic resonance imaging, echo planar magnetic resonance imaging, spin echo magnetization resonance imaging, diffusion MRI, resting-state functional MRI, magnetic resonance spectroscopy, perfusion magnetic resonance imaging, dynamic susceptibility weighted dynamic susceptibility MRI, dynamic contrast-enhanced MRI, quantitative structural MRI, magnetization transfer MRI or combinations thereof.

23. The method of any one of claims 1-21, wherein the one or more samples is taken from the subject within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, with about 26 hours, within about 27 hours, within about 28 hours, within about 29 hours, within about 30 hours, within about 31 hours, within about 32 hours, within about 33 hours, with about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, within about 48 hours, within about 49 hours, within about 50 hours, within about 60 hours, within about 61 hours, within about 62 hours, within about 63 hours, within about 64 hours, within about 65 hours, within about 66 hours, within about 67 hours, within about 68 hours, within about 69 hours, within about 70 hours, within about 71 hours, or within about 72 hours of the actual or suspected injury to the head.

24. The method of any of claims 16-19, wherein the subject has been receiving treatment for the TBI.

25. The method of any of claims 16-19, wherein the subject has not been receiving treatment for the TBI.

26. The method of any of claims 11-25, wherein the subject has a mild TBI.

27. The method of any of claims 11-25, wherein the subject has a moderate, severe or moderate to severe TBI.

28. The method of any of claims 4-27, wherein the method further comprises monitoring the subject.

29. The method of any one of claims 4-28, wherein the method further comprises administering a treatment for the TBI to the subject.

30. The method of claim 9 or 13, wherein the advanced MRI procedure identifies the location of the TBI in the brain of the subject.

31. The method of claim 30, wherein the subject is predicted of having a favorable outcome based on the location of the TBI.

32. The method of claim 30, wherein the subject is predicted of having an unfavorable outcome based on the location of the TBI.

33. The method of any of claims 1-32, wherein the one or more biomarkers comprises two or more biomarkers.

34. The method of any of claims 1-32, wherein the one or more biomarkers comprises four or more biomarkers.

35. The method of any one of claims 1 to 34, wherein the sample is selected from the group consisting of a whole blood sample, a serum sample, a cerebrospinal fluid sample, and a plasma sample.

36. The method of any one of claims 1 to 35, wherein the sample is obtained after the subject sustained an injury to the head caused by physical shaking, blunt impact by an external mechanical or other force that results in a closed or open head trauma, one or more falls, explosions or blasts or other types of blunt force trauma.

37. The method of any one of claims 1 to 35, wherein the sample is obtained after the subject has ingested or been exposed to a chemical, toxin or combination of a chemical and toxin.

38. The method of claim 37, wherein the chemical or toxin is fire, mold, asbestos, a pesticide, an insecticide, an organic solvent, a paint, a glue, a gas, an organic metal, a drug of abuse or one or more combinations thereof.

39. The method of any one of claims 1 to 35, wherein the sample is obtained from a subject that suffers from an autoimmune disease, a metabolic disorder, a brain tumor, hypoxia, a virus, meningitis, hydrocephalus or combinations thereof.

40. The method of any one of claims 1 to 39, wherein said method can be carried out on any subject without regard to factors selected from the group consisting of the subject's clinical condition, the subject's laboratory values, the subject's classification as suffering from mild, moderate, severe, or moderate to severe traumatic brain injury, the subject's exhibition of low or high levels of one or more biomarkers, and the timing of any event wherein said subject may have sustained an injury to the head.

41. The method of any one of claims 1 to 40, wherein the sample is a whole blood sample.

42. The method of any one of claims 1 to 40, wherein the sample is a serum sample.

43. The method of any one of claims 1 to 40, wherein the sample is a plasma sample.

44. The method of any one of claims 1 to 43, wherein the assay is an immunoassay.

45. The method of any one of claims 1 to 43, wherein the assay is a clinical chemistry assay.

46. The method of any one of claims 1 to 43, wherein the assay is a single molecule detection assay.

47. The method of any one of claims 4 to 7, wherein the pathophysiology of the head injury is determined by assessing whether the subject has or is suffering from at least one of cerebral hypofusion or hyperfusion, cerebral ischaemia, hyperaemia, impaired cerebrovascular CO₂-reactivity, a cerebral vasospasm, brain tissue hypoxia, oedema, oxidative stress, cell death or combinations thereof.

48. The method of claims 4 to 7 and 47, wherein the endotype of the head injury is determined by determining (i) whether the injury is axonal, neuronal or astroglial; (ii) whether there is microvascular injury; (iii) whether there is proteinopathy; (iv) whether is there inflammation; or (v) any combinations thereof.
